SEQUENCE LISTING (749 sequences)

SEQ ID NO: 1 - protein, 17 aa, synthetic construct
  REGION 1..17: note = HNP401
SGQVPWEEPYYVVKKSS

SEQ ID NO: 2 - protein, 16 aa, synthetic construct
  REGION 1..16: note = HNP402
WEYHYVDLNWTSQHPQ

SEQ ID NO: 3 - protein, 14 aa, synthetic construct
  REGION 1..14: note = HNP403
DLPDIIWDFNWETA

SEQ ID NO: 4 - protein, 20 aa, synthetic construct
  REGION 1..20: note = HNP401 with GGC linker
  MOD_RES 1: note = acetyl
SGQVPWEEPYYVVKKSSGGC

SEQ ID NO: 5 - protein, 19 aa, synthetic construct
  REGION 1..19: note = HNP402 with GGC linker
  MOD_RES 1: note = acetyl
WEYHYVDLNWTSQHPQGGC

SEQ ID NO: 6 - protein, 17 aa, synthetic construct
  REGION 1..17: note = HNP403 with GGC linker
  MOD_RES 1: note = acetyl
DLPDIIWDFNWETAGGC

SEQ ID NO: 7 - protein, 18 aa, synthetic construct
  REGION 1..18: note = HNP401-N-2 with GGC linker
  MOD_RES 1: note = acetyl
QVPWEEPYYVVKKSSGGC

SEQ ID NO: 8 - protein, 16 aa, synthetic construct
  REGION 1..16: note = HNP401-N-4 with GGC linker
  MOD_RES 1: note = acetyl
PWEEPYYVVKKSSGGC

SEQ ID NO: 9 - protein, 14 aa, synthetic construct
  REGION 1..14: note = HNP401-N-6 with GGC linker
  MOD_RES 1: note = acetyl
EEPYYVVKKSSGGC

SEQ ID NO: 10 - protein, 12 aa, synthetic construct
  REGION 1..12: note = HNP401-N-8 with GGC linker
  MOD_RES 1: note = acetyl
PYYVVKKSSGGC

SEQ ID NO: 11 - protein, 18 aa, synthetic construct
  REGION 1..18: note = HNP401-C-2 with GGC linker
  MOD_RES 1: note = acetyl
SGQVPWEEPYYVVKKGGC

SEQ ID NO: 12 - protein, 16 aa, synthetic construct
  REGION 1..16: note = HNP401-C-4 with GGC linker
  MOD_RES 1: note = acetyl
SGQVPWEEPYYVVGGC

SEQ ID NO: 13 - protein, 14 aa, synthetic construct
  REGION 1..14: note = HNP401-C-6 with GGC linker
  MOD_RES 1: note = acetyl
SGQVPWEEPYYGGC

SEQ ID NO: 14 - protein, 12 aa, synthetic construct
  REGION 1..12: note = HNP401-C-8 with GGC linker
  MOD_RES 1: note = acetyl
SGQVPWEEPGGC

SEQ ID NO: 15 - protein, 4 aa, synthetic construct
  REGION 1..4: note = linker
GGGC

SEQ ID NO: 16 - protein, 16 aa, synthetic construct
  REGION 1..16: note = HNP404
DTHAHAKPRVPAFKSV

SEQ ID NO: 17 - protein, 17 aa, synthetic construct
  REGION 1..17: note = NP41
  MOD_RES 1: note = acetyl
SHSNTQTLAKAPEHTGC

SEQ ID NO: 18 - protein, 8 aa, synthetic construct
  REGION 1..8: note = R3 Nidogen-2
KAIHPMRG

SEQ ID NO: 19 - protein, 7 aa, synthetic construct
  REGION 1..7: note = R3 Nidogen-2
GYWRNAL

SEQ ID NO: 20 - protein, 15 aa, synthetic construct
  REGION 1..15: note = HNP401-N-2
QVPWEEPYYVVKKSS

SEQ ID NO: 21 - protein, 17 aa, synthetic construct
  REGION 1..17: note = HNP401-N-2 with GG linker
QVPWEEPYYVVKKSSGG

SEQ ID NO: 22 - protein, 13 aa, synthetic construct
  REGION 1..13: note = HNP401-N-4
PWEEPYYVVKKSS

SEQ ID NO: 23 - protein, 11 aa, synthetic construct
  REGION 1..11: note = HNP401-N-6
EEPYYVVKKSS

SEQ ID NO: 24 - protein, 9 aa, synthetic construct
  REGION 1..9: note = HNP401-N-8
PYYVVKKSS

SEQ ID NO: 25 - protein, 15 aa, synthetic construct
  REGION 1..15: note = HNP401-C-2
SGQVPWEEPYYVVKK

SEQ ID NO: 26 - protein, 13 aa, synthetic construct
  REGION 1..13: note = HNP401-C-4
SGQVPWEEPYYVV

SEQ ID NO: 27 - protein, 11 aa, synthetic construct
  REGION 1..11: note = HNP401-C-6
SGQVPWEEPYY

SEQ ID NO: 28 - protein, 9 aa, synthetic construct
  REGION 1..9: note = HNP401-C-8
SGQVPWEEP

SEQ ID NO: 29 - protein, 10 aa, synthetic construct
  REGION 1..10: note = R3 Nidogen-2
ACTVHKWDNC

SEQ ID NO: 30 - protein, 10 aa, synthetic construct
  REGION 1..10: note = R3 Nidogen-2
ACEPRSLANC

SEQ ID NO: 31 - protein, 10 aa, synthetic construct
  REGION 1..10: note = R3 Nidogen-2
ACPENKSKHC

SEQ ID NO: 32 - protein, 10 aa, synthetic construct
  REGION 1..10: note = R3 Nidogen-2
ACGEGEADVC

SEQ ID NO: 33 - protein, 7 aa, synthetic construct
  REGION 1..7: note = R3 Myelin PLP
EKPSWTR

SEQ ID NO: 34 - protein, 7 aa, synthetic construct
  REGION 1..7: note = R3 Myelin PLP
DTSRTME

SEQ ID NO: 35 - protein, 7 aa, synthetic construct
  REGION 1..7: note = R3 Myelin PLP
SMWSQFR

SEQ ID NO: 36 - protein, 7 aa, synthetic construct
  REGION 1..7: note = R3 Myelin PLP
NPNWRTI

SEQ ID NO: 37 - protein, 7 aa, synthetic construct
  REGION 1..7: note = R3 Myelin PLP
FKMPIPT

SEQ ID NO: 38 - protein, 7 aa, synthetic construct
  REGION 1..7: note = R3 Myelin PLP
YPKLWTY

SEQ ID NO: 39 - protein, 7 aa, synthetic construct
  REGION 1..7: note = R3 Myelin PLP
NPSHHPR

SEQ ID NO: 40 - protein, 7 aa, synthetic construct
  REGION 1..7: note = core binding domain
PYYVVKK

SEQ ID NO: 41 - protein, 6 aa, synthetic construct
  REGION 1..6: note = N-terminal sequence
QVPWEE

SEQ ID NO: 42 - protein, 7 aa, synthetic construct
  REGION 1..7: note = R3 Myelin PLP
LTHHFAP

SEQ ID NO: 43 - protein, 7 aa, synthetic construct
  REGION 1..7: note = R3 Myelin PLP
RHNVEFS

SEQ ID NO: 44 - protein, 7 aa, synthetic construct
  REGION 1..7: note = R3 Myelin PLP
FPVVTRN

SEQ ID NO: 45 - protein, 7 aa, synthetic construct
  REGION 1..7: note = R3 Myelin PLP
LRFQIPP

SEQ ID NO: 46 - protein, 7 aa, synthetic construct
  REGION 1..7: note = R3 Myelin PLP
LALYARM

SEQ ID NO: 47 - protein, 7 aa, synthetic construct
  REGION 1..7: note = R3 Myelin PLP
GPFLFTV

SEQ ID NO: 48 - protein, 7 aa, synthetic construct
  REGION 1..7: note = R3 Myelin PLP
YTPMGTG

SEQ ID NO: 49 - protein, 7 aa, synthetic construct
  REGION 1..7: note = R3 Myelin PLP
RFELPAP

SEQ ID NO: 50 - protein, 7 aa, synthetic construct
  REGION 1..7: note = R3 Myelin PLP
HQGSRTY

SEQ ID NO: 51 - protein, 7 aa, synthetic construct
  REGION 1..7: note = R3 Myelin PLP
TNPWKPH

SEQ ID NO: 52 - protein, 7 aa, synthetic construct
  REGION 1..7: note = R3 Myelin PLP
MTPHTSC

SEQ ID NO: 53 - protein, 7 aa, synthetic construct
  REGION 1..7: note = R3 Myelin PLP
GLIRHYT

SEQ ID NO: 54 - protein, 7 aa, synthetic construct
  REGION 1..7: note = R3 Myelin PLP
ATLKPYR

SEQ ID NO: 55 - protein, 7 aa, synthetic construct
  REGION 1..7: note = R3 Myelin Basic Protein
HRLPWHH

SEQ ID NO: 56 - protein, 7 aa, synthetic construct
  REGION 1..7: note = R3 Myelin Basic Protein
DGKNTSN

SEQ ID NO: 57 - protein, 7 aa, synthetic construct
  REGION 1..7: note = R3 Myelin Basic Protein
AGFPPNT

SEQ ID NO: 58 - protein, 7 aa, synthetic construct
  REGION 1..7: note = R3 Myelin Basic Protein
QNTGLKW

SEQ ID NO: 59 - protein, 7 aa, synthetic construct
  REGION 1..7: note = R3 Myelin Basic Protein
QIGVLPS

SEQ ID NO: 60 - protein, 7 aa, synthetic construct
  REGION 1..7: note = R3 Myelin Basic Protein
DPWVMLR

SEQ ID NO: 61 - protein, 7 aa, synthetic construct
  REGION 1..7: note = R3 Myelin Basic Protein
SPDRTSL

SEQ ID NO: 62 - protein, 7 aa, synthetic construct
  REGION 1..7: note = R3 Myelin Basic Protein
MQTQGRV

SEQ ID NO: 63 - protein, 7 aa, synthetic construct
  REGION 1..7: note = R3 Myelin Basic Protein
LRPISHE

SEQ ID NO: 64 - protein, 7 aa, synthetic construct
  REGION 1..7: note = R3 Myelin Basic Protein
AWSTSSV

SEQ ID NO: 65 - protein, 10 aa, synthetic construct
  REGION 1..10: note = R3 Myelin Basic Protein
AGQLMRCANC

SEQ ID NO: 66 - protein, 7 aa, synthetic construct
  REGION 1..7: note = R3 Myelin Basic Protein
WTMKPNY

SEQ ID NO: 67 - protein, 7 aa, synthetic construct
  REGION 1..7: note = R3 Myelin Basic Protein
ILLPSLN

SEQ ID NO: 68 - protein, 7 aa, synthetic construct
  REGION 1..7: note = R3 Myelin Basic Protein
SKSDAWR

SEQ ID NO: 69 - protein, 7 aa, synthetic construct
  REGION 1..7: note = R3 Myelin Basic Protein
AITSRNA

SEQ ID NO: 70 - protein, 7 aa, synthetic construct
  REGION 1..7: note = R3 Myelin Basic Protein
SVLRMLN

SEQ ID NO: 71 - protein, 7 aa, synthetic construct
  REGION 1..7: note = R3 Myelin Basic Protein
LPMRILT

SEQ ID NO: 72 - protein, 7 aa, synthetic construct
  REGION 1..7: note = R3 Myelin Basic Protein
EHGYYKV

SEQ ID NO: 73 - protein, 7 aa, synthetic construct
  REGION 1..7: note = R3 Myelin Basic Protein
HPSTWHK

SEQ ID NO: 74 - protein, 7 aa, synthetic construct
  REGION 1..7: note = R3 Myelin Basic Protein
NDTKTPS

SEQ ID NO: 75 - protein, 12 aa, synthetic construct
  REGION 1..12: note = R3 Human MPZ
GDERIVRTLSHN

SEQ ID NO: 76 - protein, 7 aa, synthetic construct
  REGION 1..7: note = R3 Human MPZ
GLTPKLF

SEQ ID NO: 77 - protein, 7 aa, synthetic construct
  REGION 1..7: note = R3 Human MPZ
RVPHSRT

SEQ ID NO: 78 - protein, 7 aa, synthetic construct
  REGION 1..7: note = R3 Human MPZ
TESLFPH

SEQ ID NO: 79 - protein, 7 aa, synthetic construct
  REGION 1..7: note = R3 Human MPZ
GPLRMAT

SEQ ID NO: 80 - protein, 7 aa, synthetic construct
  REGION 1..7: note = R3 Human MPZ
DRNSVFW

SEQ ID NO: 81 - protein, 7 aa, synthetic construct
  REGION 1..7: note = R3 Human MPZ
SAHAKHH

SEQ ID NO: 82 - protein, 7 aa, synthetic construct
  REGION 1..7: note = R3 Human MPZ
KMAGHTV

SEQ ID NO: 83 - protein, 5 aa, synthetic construct
  REGION 1..5: note = core binding domain
PYYVV

SEQ ID NO: 84 - protein, 7 aa, synthetic construct
  REGION 1..7: note = R3 Human MPZ
TMASPAK

SEQ ID NO: 85 - protein, 7 aa, synthetic construct
  REGION 1..7: note = R3 Human MPZ
VLTKPMP

SEQ ID NO: 86 - protein, 7 aa, synthetic construct
  REGION 1..7: note = R3 Human MPZ
LSSPLSL

SEQ ID NO: 87 - protein, 7 aa, synthetic construct
  REGION 1..7: note = R3 Human MPZ
WPSISRP

SEQ ID NO: 88 - protein, 7 aa, synthetic construct
  REGION 1..7: note = R3 Human MPZ
SHSPGNK

SEQ ID NO: 89 - protein, 7 aa, synthetic construct
  REGION 1..7: note = R3 Human MPZ
ETTRNYG

SEQ ID NO: 90 - protein, 7 aa, synthetic construct
  REGION 1..7: note = R3 Human MPZ
IKPPYTH

SEQ ID NO: 91 - protein, 7 aa, synthetic construct
  REGION 1..7: note = R3 Human MPZ
VLKNNPT

SEQ ID NO: 92 - protein, 7 aa, synthetic construct
  REGION 1..7: note = R3 Human MPZ
DPITRLK

SEQ ID NO: 93 - protein, 7 aa, synthetic construct
  REGION 1..7: note = R3 Human MPZ
SHLASRP

SEQ ID NO: 94 - protein, 7 aa, synthetic construct
  REGION 1..7: note = R3 Human MPZ
TSPWELR

SEQ ID NO: 95 - protein, 7 aa, synthetic construct
  REGION 1..7: note = R3 Human MPZ
ALPLSAS

SEQ ID NO: 96 - protein, 11 aa, synthetic construct
  REGION 1..11: note = HNP401-C-8 (with GG linker)
SGQVPWEEPGG

SEQ ID NO: 97 - protein, 13 aa, synthetic construct
  REGION 1..13: note = HNP401-C-6 (with GG linker)
SGQVPWEEPYYGG

SEQ ID NO: 98 - protein, 15 aa, synthetic construct
  REGION 1..15: note = HNP401-C-4 (with GG linker)
SGQVPWEEPYYVVGG

SEQ ID NO: 99 - protein, 17 aa, synthetic construct
  REGION 1..17: note = HNP401-C-2 (with GG linker)
SGQVPWEEPYYVVKKGG

SEQ ID NO: 100 - protein, 11 aa, synthetic construct
  REGION 1..11: note = HNP401-N-8 (with GG linker)
PYYVVKKSSGG

SEQ ID NO: 101 - protein, 13 aa, synthetic construct
  REGION 1..13: note = HNP401-N-6 (with GG linker)
EEPYYVVKKSSGG

SEQ ID NO: 102 - protein, 15 aa, synthetic construct
  REGION 1..15: note = HNP401-N-4 (with GG linker)
PWEEPYYVVKKSSGG

SEQ ID NO: 103 - protein, 9 aa, synthetic construct
  REGION 1..9: note = R3 Human MPZ
CLKQQTGDC

SEQ ID NO: 104 - protein, 12 aa, synthetic construct
  REGION 1..12: note = R3 Human MPZ
EGHVWSEYTWGT

SEQ ID NO: 105 - protein, 7 aa, synthetic construct
  REGION 1..7: note = R3 Human MPZ
FMSDVDH

SEQ ID NO: 106 - protein, 7 aa, synthetic construct
  REGION 1..7: note = R3 Human MPZ
ISSVTVV

SEQ ID NO: 107 - protein, 7 aa, synthetic construct
  REGION 1..7: note = R3 Human MPZ
KQPAMFN

SEQ ID NO: 108 - protein, 7 aa, synthetic construct
  REGION 1..7: note = R3 Human MPZ
LISPTAR

SEQ ID NO: 109 - protein, 7 aa, synthetic construct
  REGION 1..7: note = R3 Human MPZ
LVLQTTP

SEQ ID NO: 110 - protein, 7 aa, synthetic construct
  REGION 1..7: note = R3 Human MPZ
NPTSEGA

SEQ ID NO: 111 - protein, 7 aa, synthetic construct
  REGION 1..7: note = R3 Human MPZ
THSTDLS

SEQ ID NO: 112 - protein, 12 aa, synthetic construct
  REGION 1..12: note = R3 Human MPZ
TTNVTEIERESY

SEQ ID NO: 113 - protein, 7 aa, synthetic construct
  REGION 1..7: note = R3 Human MPZ
YAPWRVF

SEQ ID NO: 114 - protein, 7 aa, synthetic construct
  REGION 1..7: note = R3 Human MPZ
YGQSITE

SEQ ID NO: 115 - protein, 9 aa, synthetic construct
  REGION 1..9: note = R3 Human MPZ
CDIKDANSC

SEQ ID NO: 116 - protein, 9 aa, synthetic construct
  REGION 1..9: note = R3 Human MPZ
CIKGEDNQC

SEQ ID NO: 117 - protein, 7 aa, synthetic construct
  REGION 1..7: note = R3 Human MPZ
HNQLLYT

SEQ ID NO: 118 - protein, 7 aa, synthetic construct
  REGION 1..7: note = R3 Human MPZ
IEQVGWR

SEQ ID NO: 119 - protein, 7 aa, synthetic construct
  REGION 1..7: note = R3 Human MPZ
KTPILAS

SEQ ID NO: 120 - protein, 7 aa, synthetic construct
  REGION 1..7: note = R3 Human MPZ
LWAKRDA

SEQ ID NO: 121 - protein, 7 aa, synthetic construct
  REGION 1..7: note = R3 Human MPZ
NPISPRN

SEQ ID NO: 122 - protein, 7 aa, synthetic construct
  REGION 1..7: note = R3 Human MPZ
NTASMTT

SEQ ID NO: 123 - protein, 7 aa, synthetic construct
  REGION 1..7: note = R3 Human MPZ
SAHSVVE

SEQ ID NO: 124 - protein, 7 aa, synthetic construct
  REGION 1..7: note = R3 Human MPZ
YSSLTSN

SEQ ID NO: 125 - protein, 7 aa, synthetic construct
  REGION 1..7: note = R3 Human MPZ
AWTMRAS

SEQ ID NO: 126 - protein, 9 aa, synthetic construct
  REGION 1..9: note = R3 Human MPZ
CERIGPREC

SEQ ID NO: 127 - protein, 9 aa, synthetic construct
  REGION 1..9: note = R3 Human MPZ
CQKWFTFAC

SEQ ID NO: 128 - protein, 9 aa, synthetic construct
  REGION 1..9: note = R3 Human MPZ
CSNALNKAC

SEQ ID NO: 129 - protein, 7 aa, synthetic construct
  REGION 1..7: note = R3 Human MPZ
FSPRDVS

SEQ ID NO: 130 - protein, 7 aa, synthetic construct
  REGION 1..7: note = R3 Human MPZ
FSTTGRG

SEQ ID NO: 131 - protein, 7 aa, synthetic construct
  REGION 1..7: note = R3 Human MPZ
HHPLFID

SEQ ID NO: 132 - protein, 7 aa, synthetic construct
  REGION 1..7: note = R3 Human MPZ
LVNPFPE

SEQ ID NO: 133 - protein, 7 aa, synthetic construct
  REGION 1..7: note = R3 Human MPZ
YEGERGA

SEQ ID NO: 134 - protein, 9 aa, synthetic construct
  REGION 1..9: note = R3 Human MPZ
CEDRQRSMC

SEQ ID NO: 135 - protein, 9 aa, synthetic construct
  REGION 1..9: note = R3 Human MPZ
CGEKIMSLC

SEQ ID NO: 136 - protein, 7 aa, synthetic construct
  REGION 1..7: note = R3 Human MPZ
DRYLLNQ

SEQ ID NO: 137 - protein, 7 aa, synthetic construct
  REGION 1..7: note = R3 Human MPZ
FPSYTIS

SEQ ID NO: 138 - protein, 7 aa, synthetic construct
  REGION 1..7: note = R3 Human MPZ
GGSKVWG

SEQ ID NO: 139 - protein, 7 aa, synthetic construct
  REGION 1..7: note = R3 Human MPZ
GLWRHTM

SEQ ID NO: 140 - protein, 7 aa, synthetic construct
  REGION 1..7: note = R3 Human MPZ
KRNLWDI

SEQ ID NO: 141 - protein, 7 aa, synthetic construct
  REGION 1..7: note = R3 Human MPZ
SPNTNSH

SEQ ID NO: 142 - protein, 7 aa, synthetic construct
  REGION 1..7: note = R3 Human MPZ
TLLHYSA

SEQ ID NO: 143 - protein, 7 aa, synthetic construct
  REGION 1..7: note = R3 Human MPZ
VPLAYLR

SEQ ID NO: 144 - protein, 7 aa, synthetic construct
  REGION 1..7: note = R3 Human MPZ
VVPLKWI

SEQ ID NO: 145 - protein, 9 aa, synthetic construct
  REGION 1..9: note = R3 Human MPZ
CRVGVTSAC

SEQ ID NO: 146 - protein, 7 aa, synthetic construct
  REGION 1..7: note = R3 Human MPZ
FALPAGL

SEQ ID NO: 147 - protein, 7 aa, synthetic construct
  REGION 1..7: note = R3 Human MPZ
FRVTQTA

SEQ ID NO: 148 - protein, 7 aa, synthetic construct
  REGION 1..7: note = R3 Human MPZ
HPLMVPS

SEQ ID NO: 149 - protein, 7 aa, synthetic construct
  REGION 1..7: note = R3 Human MPZ
LPNANNL

SEQ ID NO: 150 - protein, 7 aa, synthetic construct
  REGION 1..7: note = R3 Human MPZ
MIHTRQT

SEQ ID NO: 151 - protein, 7 aa, synthetic construct
  REGION 1..7: note = R3 Human MPZ
SETESYM

SEQ ID NO: 152 - protein, 7 aa, synthetic construct
  REGION 1..7: note = R3 Human MPZ
SNALSYF

SEQ ID NO: 153 - protein, 7 aa, synthetic construct
  REGION 1..7: note = R3 Human MPZ
SYLPPFI

SEQ ID NO: 154 - protein, 7 aa, synthetic construct
  REGION 1..7: note = R3 Human MPZ
VHTPYRS

SEQ ID NO: 155 - protein, 7 aa, synthetic construct
  REGION 1..7: note = R3 Human MPZ
WPRPIQI

SEQ ID NO: 156 - protein, 7 aa, synthetic construct
  REGION 1..7: note = R3 Human MPZ
WTSVATA

SEQ ID NO: 157 - protein, 7 aa, synthetic construct
  REGION 1..7: note = R3 Human MPZ
ALPISYL

SEQ ID NO: 158 - protein, 7 aa, synthetic construct
  REGION 1..7: note = R3 Human MPZ
FEHMAVT

SEQ ID NO: 159 - protein, 7 aa, synthetic construct
  REGION 1..7: note = R3 Human MPZ
NASVPPK

SEQ ID NO: 160 - protein, 7 aa, synthetic construct
  REGION 1..7: note = R3 Human MPZ
SGHGAFR

SEQ ID NO: 161 - protein, 7 aa, synthetic construct
  REGION 1..7: note = R3 Human MPZ
TTLVVTA

SEQ ID NO: 162 - protein, 9 aa, synthetic construct
  REGION 1..9: note = R3 Human MPZ
CPDAARNSC

SEQ ID NO: 163 - protein, 9 aa, synthetic construct
  REGION 1..9: note = R3 Human MPZ
CYSSVADMC

SEQ ID NO: 164 - protein, 7 aa, synthetic construct
  REGION 1..7: note = R3 Human MPZ
LSPPRIM

SEQ ID NO: 165 - protein, 7 aa, synthetic construct
  REGION 1..7: note = R3 Human MPZ
WMTNLDP

SEQ ID NO: 166 - protein, 7 aa, synthetic construct
  REGION 1..7: note = R3 Human MPZ
KLPTLSV

SEQ ID NO: 167 - protein, 7 aa, synthetic construct
  REGION 1..7: note = R3 Human MPZ
AMSNLSY

SEQ ID NO: 168 - protein, 7 aa, synthetic construct
  REGION 1..7: note = R3 Human MPZ
HPFPRFD

SEQ ID NO: 169 - protein, 7 aa, synthetic construct
  REGION 1..7: note = R3 Human MPZ
LPAESHW

SEQ ID NO: 170 - protein, 7 aa, synthetic construct
  REGION 1..7: note = R3 Human MPZ
STHEWRT

SEQ ID NO: 171 - protein, 7 aa, synthetic construct
  REGION 1..7: note = R3 Human MPZ
TTWPNTA

SEQ ID NO: 172 - protein, 7 aa, synthetic construct
  REGION 1..7: note = R3 Human MPZ
YMKHSPG

SEQ ID NO: 173 - protein, 12 aa, synthetic construct
  REGION 1..12: note = R3 Human MPZ
AYLEDWRSMSTR

SEQ ID NO: 174 - protein, 7 aa, synthetic construct
  REGION 1..7: note = R3 Human MPZ
GPVVSGM

SEQ ID NO: 175 - protein, 7 aa, synthetic construct
  REGION 1..7: note = R3 Human MPZ
HLDSRRH

SEQ ID NO: 176 - protein, 7 aa, synthetic construct
  REGION 1..7: note = R3 Human MPZ
LVLSNPR

SEQ ID NO: 177 - protein, 7 aa, synthetic construct
  REGION 1..7: note = R3 Human MPZ
NMSHSNR

SEQ ID NO: 178 - protein, 7 aa, synthetic construct
  REGION 1..7: note = R3 Human MPZ
SVAYVPV

SEQ ID NO: 179 - protein, 7 aa, synthetic construct
  REGION 1..7: note = R3 Human MPZ
YPQARTS

SEQ ID NO: 180 - protein, 7 aa, synthetic construct
  REGION 1..7: note = R3 Human MPZ
MYHDSVS

SEQ ID NO: 181 - protein, 12 aa, synthetic construct
  REGION 1..12: note = R3 Human MPZ
ADNVLRRALENI

SEQ ID NO: 182 - protein, 7 aa, synthetic construct
  REGION 1..7: note = R3 Human MPZ
QIAHLEY

SEQ ID NO: 183 - protein, 12 aa, synthetic construct
  REGION 1..12: note = R3 Human MPZ
GMVSPHHSVYRH

SEQ ID NO: 184 - protein, 7 aa, synthetic construct
  REGION 1..7: note = R3 Human MPZ
GSWSSGF

SEQ ID NO: 185 - protein, 12 aa, synthetic construct
  REGION 1..12: note = R3 Human MPZ
NGAYSLAIRYTS

SEQ ID NO: 186 - protein, 12 aa, synthetic construct
  REGION 1..12: note = R3 Human MPZ
YTPNWHFRWMPA

SEQ ID NO: 187 - protein, 12 aa, synthetic construct
  REGION 1..12: note = R3 Human MPZ
GEWRARIDQDVS

SEQ ID NO: 188 - protein, 7 aa, synthetic construct
  REGION 1..7: note = R3 Myelin Basic Protein
AGSLLSL

SEQ ID NO: 189 - protein, 7 aa, synthetic construct
  REGION 1..7: note = R3 Myelin Basic Protein
AIPPRKL

SEQ ID NO: 190 - protein, 7 aa, synthetic construct
  REGION 1..7: note = R3 Myelin Basic Protein
AISWKGF

SEQ ID NO: 191 - protein, 7 aa, synthetic construct
  REGION 1..7: note = R3 Myelin Basic Protein
AKFTMWV

SEQ ID NO: 192 - protein, 7 aa, synthetic construct
  REGION 1..7: note = R3 Myelin Basic Protein
ALRDTRV

SEQ ID NO: 193 - protein, 7 aa, synthetic construct
  REGION 1..7: note = R3 Myelin Basic Protein
ALYNQMT

SEQ ID NO: 194 - protein, 7 aa, synthetic construct
  REGION 1..7: note = R3 Myelin Basic Protein
AQFLTIY

SEQ ID NO: 195 - protein, 7 aa, synthetic construct
  REGION 1..7: note = R3 Myelin Basic Protein
AQLGAFR

SEQ ID NO: 196 - protein, 7 aa, synthetic construct
  REGION 1..7: note = R3 Myelin Basic Protein
ARVPNPL

SEQ ID NO: 197 - protein, 9 aa, synthetic construct
  REGION 1..9: note = R3 Myelin Basic Protein
CTMTNQYDC

SEQ ID NO: 198 - protein, 7 aa, synthetic construct
  REGION 1..7: note = R3 Myelin Basic Protein
DSQEKSV

SEQ ID NO: 199 - protein, 7 aa, synthetic construct
  REGION 1..7: note = R3 Myelin Basic Protein
DVRIWTL

SEQ ID NO: 200 - protein, 7 aa, synthetic construct
  REGION 1..7: note = R3 Myelin Basic Protein
ELKSLRF

SEQ ID NO: 201 - protein, 7 aa, synthetic construct
  REGION 1..7: note = R3 Myelin Basic Protein
ESKSPRE

SEQ ID NO: 202 - protein, 7 aa, synthetic construct
  REGION 1..7: note = R3 Myelin Basic Protein
ETHYKIA

SEQ ID NO: 203 - protein, 7 aa, synthetic construct
  REGION 1..7: note = R3 Myelin Basic Protein
FDLLARK

SEQ ID NO: 204 - protein, 7 aa, synthetic construct
  REGION 1..7: note = R3 Myelin Basic Protein
FQLSYET

SEQ ID NO: 205 - protein, 7 aa, synthetic construct
  REGION 1..7: note = R3 Myelin Basic Protein
GEPALVT

SEQ ID NO: 206 - protein, 7 aa, synthetic construct
  REGION 1..7: note = R3 Myelin Basic Protein
GPRVTPH

SEQ ID NO: 207 - protein, 7 aa, synthetic construct
  REGION 1..7: note = R3 Myelin Basic Protein
GQWWKML

SEQ ID NO: 208 - protein, 7 aa, synthetic construct
  REGION 1..7: note = R3 Myelin Basic Protein
GRHTDVV

SEQ ID NO: 209 - protein, 7 aa, synthetic construct
  REGION 1..7: note = R3 Myelin Basic Protein
GSPYGWG

SEQ ID NO: 210 - protein, 7 aa, synthetic construct
  REGION 1..7: note = R3 Myelin Basic Protein
GVAEVGL

SEQ ID NO: 211 - protein, 7 aa, synthetic construct
  REGION 1..7: note = R3 Myelin Basic Protein
HAHWARL

SEQ ID NO: 212 - protein, 7 aa, synthetic construct
  REGION 1..7: note = R3 Myelin Basic Protein
HFTFFPI

SEQ ID NO: 213 - protein, 7 aa, synthetic construct
  REGION 1..7: note = R3 Myelin Basic Protein
HPWSHPN

SEQ ID NO: 214 - protein, 7 aa, synthetic construct
  REGION 1..7: note = R3 Myelin Basic Protein
HQMRTML

SEQ ID NO: 215 - protein, 7 aa, synthetic construct
  REGION 1..7: note = R3 Myelin Basic Protein
HQPLFPR

SEQ ID NO: 216 - protein, 7 aa, synthetic construct
  REGION 1..7: note = R3 Myelin Basic Protein
HRTHLHQ

SEQ ID NO: 217 - protein, 7 aa, synthetic construct
  REGION 1..7: note = R3 Myelin Basic Protein
HSGPLLP

SEQ ID NO: 218 - protein, 7 aa, synthetic construct
  REGION 1..7: note = R3 Myelin Basic Protein
HSSATMS

SEQ ID NO: 219 - protein, 7 aa, synthetic construct
  REGION 1..7: note = R3 Myelin Basic Protein
IGTRWHQ

SEQ ID NO: 220 - protein, 7 aa, synthetic construct
  REGION 1..7: note = R3 Myelin Basic Protein
INFPFAV

SEQ ID NO: 221 - protein, 7 aa, synthetic construct
  REGION 1..7: note = R3 Myelin Basic Protein
IPIPYRT

SEQ ID NO: 222 - protein, 7 aa, synthetic construct
  REGION 1..7: note = R3 Myelin Basic Protein
IPYSQMP

SEQ ID NO: 223 - protein, 7 aa, synthetic construct
  REGION 1..7: note = R3 Myelin Basic Protein
KIITTKS

SEQ ID NO: 224 - protein, 7 aa, synthetic construct
  REGION 1..7: note = R3 Myelin Basic Protein
KMSLAPP

SEQ ID NO: 225 - protein, 7 aa, synthetic construct
  REGION 1..7: note = R3 Myelin Basic Protein
KVHHLAR

SEQ ID NO: 226 - protein, 7 aa, synthetic construct
  REGION 1..7: note = R3 Myelin Basic Protein
LELHQNV

SEQ ID NO: 227 - protein, 7 aa, synthetic construct
  REGION 1..7: note = R3 Myelin Basic Protein
LITHPQL

SEQ ID NO: 228 - protein, 7 aa, synthetic construct
  REGION 1..7: note = R3 Myelin Basic Protein
LMLPQLE

SEQ ID NO: 229 - protein, 7 aa, synthetic construct
  REGION 1..7: note = R3 Myelin Basic Protein
LVPPVNH

SEQ ID NO: 230 - protein, 7 aa, synthetic construct
  REGION 1..7: note = R3 Myelin Basic Protein
MNSPLAQ

SEQ ID NO: 231 - protein, 7 aa, synthetic construct
  REGION 1..7: note = R3 Myelin Basic Protein
MQRLGAD

SEQ ID NO: 232 - protein, 7 aa, synthetic construct
  REGION 1..7: note = R3 Myelin Basic Protein
NGPNSLQ

SEQ ID NO: 233 - protein, 7 aa, synthetic construct
  REGION 1..7: note = R3 Myelin Basic Protein
NGVSSSL

SEQ ID NO: 234 - protein, 7 aa, synthetic construct
  REGION 1..7: note = R3 Myelin Basic Protein
NLTYLRF

SEQ ID NO: 235 - protein, 7 aa, synthetic construct
  REGION 1..7: note = R3 Myelin Basic Protein
QGAYVRP

SEQ ID NO: 236 - protein, 7 aa, synthetic construct
  REGION 1..7: note = R3 Myelin Basic Protein
QKALPQT

SEQ ID NO: 237 - protein, 7 aa, synthetic construct
  REGION 1..7: note = R3 Myelin Basic Protein
QTSIMSG

SEQ ID NO: 238 - protein, 7 aa, synthetic construct
  REGION 1..7: note = R3 Myelin Basic Protein
QYWDILG

SEQ ID NO: 239 - protein, 7 aa, synthetic construct
  REGION 1..7: note = R3 Myelin Basic Protein
RGHPSQL

SEQ ID NO: 240 - protein, 7 aa, synthetic construct
  REGION 1..7: note = R3 Myelin Basic Protein
RSRKHRP

SEQ ID NO: 241 - protein, 7 aa, synthetic construct
  REGION 1..7: note = R3 Myelin Basic Protein
RTTDWWT

SEQ ID NO: 242 - protein, 7 aa, synthetic construct
  REGION 1..7: note = R3 Myelin Basic Protein
RVIPSTT

SEQ ID NO: 243 - protein, 7 aa, synthetic construct
  REGION 1..7: note = R3 Myelin Basic Protein
SAVLMPP

SEQ ID NO: 244 - protein, 7 aa, synthetic construct
  REGION 1..7: note = R3 Myelin Basic Protein
SEGLTLL

SEQ ID NO: 245 - protein, 7 aa, synthetic construct
  REGION 1..7: note = R3 Myelin Basic Protein
SGPFPLV

SEQ ID NO: 246 - protein, 7 aa, synthetic construct
  REGION 1..7: note = R3 Myelin Basic Protein
SIQTSAM

SEQ ID NO: 247 - protein, 7 aa, synthetic construct
  REGION 1..7: note = R3 Myelin Basic Protein
SKLAGFP

SEQ ID NO: 248 - protein, 7 aa, synthetic construct
  REGION 1..7: note = R3 Myelin Basic Protein
SKPHASH

SEQ ID NO: 249 - protein, 7 aa, synthetic construct
  REGION 1..7: note = R3 Myelin Basic Protein
SLDHLRL

SEQ ID NO: 250 - protein, 7 aa, synthetic construct
  REGION 1..7: note = R3 Myelin Basic Protein
SLKTTNP

SEQ ID NO: 251 - protein, 7 aa, synthetic construct
  REGION 1..7: note = R3 Myelin Basic Protein
SLWQTLR

SEQ ID NO: 252 - protein, 7 aa, synthetic construct
  REGION 1..7: note = R3 Myelin Basic Protein
SLWTHRT

SEQ ID NO: 253 - protein, 7 aa, synthetic construct
  REGION 1..7: note = R3 Myelin Basic Protein
SNAHTLP

SEQ ID NO: 254 - protein, 7 aa, synthetic construct
  REGION 1..7: note = R3 Myelin Basic Protein
SPKPDIP

SEQ ID NO: 255 - protein, 7 aa, synthetic construct
  REGION 1..7: note = R3 Myelin Basic Protein
SRHLPPL

SEQ ID NO: 256 - protein, 7 aa, synthetic construct
  REGION 1..7: note = R3 Myelin Basic Protein
SSFYVAY

SEQ ID NO: 257 - protein, 7 aa, synthetic construct
  REGION 1..7: note = R3 Myelin Basic Protein
SSMRYNS

SEQ ID NO: 258 - protein, 7 aa, synthetic construct
  REGION 1..7: note = R3 Myelin Basic Protein
SVGLPTK

SEQ ID NO: 259 - protein, 7 aa, synthetic construct
  REGION 1..7: note = R3 Myelin Basic Protein
SYRSMAP

SEQ ID NO: 260 - protein, 7 aa, synthetic construct
  REGION 1..7: note = R3 Myelin Basic Protein
SYVTVFR

SEQ ID NO: 261 - protein, 7 aa, synthetic construct
  REGION 1..7: note = R3 Myelin Basic Protein
TGPSSAV

SEQ ID NO: 262 - protein, 7 aa, synthetic construct
  REGION 1..7: note = R3 Myelin Basic Protein
TGSVSKQ

SEQ ID NO: 263 - protein, 7 aa, synthetic construct
  REGION 1..7: note = R3 Myelin Basic Protein
THKHMMT

SEQ ID NO: 264 - protein, 7 aa, synthetic construct
  REGION 1..7: note = R3 Myelin Basic Protein
TIRVAHT

SEQ ID NO: 265 - protein, 7 aa, synthetic construct
  REGION 1..7: note = R3 Myelin Basic Protein
TPGRATL

SEQ ID NO: 266 - protein, 7 aa, synthetic construct
  REGION 1..7: note = R3 Myelin Basic Protein
TPVLWLP

SEQ ID NO: 267 - protein, 7 aa, synthetic construct
  REGION 1..7: note = R3 Myelin Basic Protein
TPVRLNT

SEQ ID NO: 268 - protein, 7 aa, synthetic construct
  REGION 1..7: note = R3 Myelin Basic Protein
TRIPILL

SEQ ID NO: 269 - protein, 7 aa, synthetic construct
  REGION 1..7: note = R3 Myelin Basic Protein
TTTQGHP

SEQ ID NO: 270 - protein, 7 aa, synthetic construct
  REGION 1..7: note = R3 Myelin Basic Protein
VGTFWTR

SEQ ID NO: 271 - protein, 7 aa, synthetic construct
  REGION 1..7: note = R3 Myelin Basic Protein
VIRTAAM

SEQ ID NO: 272 - protein, 7 aa, synthetic construct
  REGION 1..7: note = R3 Myelin Basic Protein
VLSPYHN

SEQ ID NO: 273 - protein, 7 aa, synthetic construct
  REGION 1..7: note = R3 Myelin Basic Protein
VRGTPNH

SEQ ID NO: 274 - protein, 7 aa, synthetic construct
  REGION 1..7: note = R3 Myelin Basic Protein
WDLPQNR

SEQ ID NO: 275 - protein, 7 aa, synthetic construct
  REGION 1..7: note = R3 Myelin Basic Protein
WDLRHSK

SEQ ID NO: 276 - protein, 7 aa, synthetic construct
  REGION 1..7: note = R3 Myelin Basic Protein
WTYHPTT

SEQ ID NO: 277 - protein, 7 aa, synthetic construct
  REGION 1..7: note = R3 Myelin Basic Protein
WVPDRSL

SEQ ID NO: 278 - protein, 7 aa, synthetic construct
  REGION 1..7: note = R3 Myelin Basic Protein
YPTYLSY

SEQ ID NO: 279 - protein, 7 aa, synthetic construct
  REGION 1..7: note = R3 Myelin Basic Protein
YPYVPIY

SEQ ID NO: 280 - protein, 7 aa, synthetic construct
  REGION 1..7: note = R3 Myelin Basic Protein
YSSRLND

SEQ ID NO: 281 - protein, 7 aa, synthetic construct
  REGION 1..7: note = R3 Myelin Basic Protein
YTASPRW

SEQ ID NO: 282 - protein, 7 aa, synthetic construct
  REGION 1..7: note = R3 Myelin Basic Protein
YVNRVKQ

SEQ ID NO: 283 - protein, 7 aa, synthetic construct
  REGION 1..7: note = R3 Myelin Basic Protein
AAGQRSF

SEQ ID NO: 284 - protein, 7 aa, synthetic construct
  REGION 1..7: note = R3 Myelin Basic Protein
AILTRPP

SEQ ID NO: 285 - protein, 7 aa, synthetic construct
  REGION 1..7: note = R3 Myelin Basic Protein
ALGTRPL

SEQ ID NO: 286 - protein, 7 aa, synthetic construct
  REGION 1..7: note = R3 Myelin Basic Protein
ASNTSKG

SEQ ID NO: 287 - protein, 7 aa, synthetic construct
  REGION 1..7: note = R3 Myelin Basic Protein
AVRQTTM

SEQ ID NO: 288 - protein, 9 aa, synthetic construct
  REGION 1..9: note = R3 Myelin Basic Protein
CEDSDKSVC

SEQ ID NO: 289 - protein, 9 aa, synthetic construct
  REGION 1..9: note = R3 Myelin Basic Protein
CNRVPFKQC

SEQ ID NO: 290 - protein, 9 aa, synthetic construct
  REGION 1..9: note = R3 Myelin Basic Protein
CPRMNNPLC

SEQ ID NO: 291 - protein, 7 aa, synthetic construct
  REGION 1..7: note = R3 Myelin Basic Protein
DVVKDVI

SEQ ID NO: 292 - protein, 7 aa, synthetic construct
  REGION 1..7: note = R3 Myelin Basic Protein
ESPYPHS

SEQ ID NO: 293 - protein, 7 aa, synthetic construct
  REGION 1..7: note = R3 Myelin Basic Protein
FKLPWAS

SEQ ID NO: 294 - protein, 7 aa, synthetic construct
  REGION 1..7: note = R3 Myelin Basic Protein
FLNQAPT

SEQ ID NO: 295 - protein, 7 aa, synthetic construct
  REGION 1..7: note = R3 Myelin Basic Protein
FSAGVGK

SEQ ID NO: 296 - protein, 12 aa, synthetic construct
  REGION 1..12: note = R3 Myelin Basic Protein
GHSHYILGKPTA

SEQ ID NO: 297 - protein, 7 aa, synthetic construct
  REGION 1..7: note = R3 Myelin Basic Protein
GPITHAI

SEQ ID NO: 298 - protein, 7 aa, synthetic construct
  REGION 1..7: note = R3 Myelin Basic Protein
HFLRPSD

SEQ ID NO: 299 - protein, 7 aa, synthetic construct
  REGION 1..7: note = R3 Myelin Basic Protein
HLLKTHS

SEQ ID NO: 300 - protein, 7 aa, synthetic construct
  REGION 1..7: note = R3 Myelin Basic Protein
HMAGVNQ

SEQ ID NO: 301 - protein, 7 aa, synthetic construct
  REGION 1..7: note = R3 Myelin Basic Protein
HPDTLPQ

SEQ ID NO: 302 - protein, 7 aa, synthetic construct
  REGION 1..7: note = R3 Myelin Basic Protein
HPTGTPT

SEQ ID NO: 303 - protein, 7 aa, synthetic construct
  REGION 1..7: note = R3 Myelin Basic Protein
HTGLRSL

SEQ ID NO: 304 - protein, 7 aa, synthetic construct
  REGION 1..7: note = R3 Myelin Basic Protein
KGQPLFR

SEQ ID NO: 305 - protein, 7 aa, synthetic construct
  REGION 1..7: note = R3 Myelin Basic Protein
KYRHGAE

SEQ ID NO: 306 - protein, 7 aa, synthetic construct
  REGION 1..7: note = R3 Myelin Basic Protein
LFRRVTE

SEQ ID NO: 307 - protein, 7 aa, synthetic construct
  REGION 1..7: note = R3 Myelin Basic Protein
LTKSLNH

SEQ ID NO: 308 - protein, 7 aa, synthetic construct
  REGION 1..7: note = R3 Myelin Basic Protein
MPLLELP

SEQ ID NO: 309 - protein, 7 aa, synthetic construct
  REGION 1..7: note = R3 Myelin Basic Protein
MTNHGNA

SEQ ID NO: 310 - protein, 7 aa, synthetic construct
  REGION 1..7: note = R3 Myelin Basic Protein
NLKLWTR

SEQ ID NO: 311 - protein, 7 aa, synthetic construct
  REGION 1..7: note = R3 Myelin Basic Protein
NLLQGVM

SEQ ID NO: 312 - protein, 7 aa, synthetic construct
  REGION 1..7: note = R3 Myelin Basic Protein
NMYPRVT

SEQ ID NO: 313 - protein, 7 aa, synthetic construct
  REGION 1..7: note = R3 Myelin Basic Protein
NSSPHQI

SEQ ID NO: 314 - protein, 7 aa, synthetic construct
  REGION 1..7: note = R3 Myelin Basic Protein
QGTLFDT

SEQ ID NO: 315 - protein, 7 aa, synthetic construct
  REGION 1..7: note = R3 Myelin Basic Protein
QPIDSTS

SEQ ID NO: 316 - protein, 7 aa, synthetic construct
  REGION 1..7: note = R3 Myelin Basic Protein
SEPGTVR

SEQ ID NO: 317 - protein, 7 aa, synthetic construct
  REGION 1..7: note = R3 Myelin Basic Protein
SGNLKKA

SEQ ID NO: 318 - protein, 12 aa, synthetic construct
  REGION 1..12: note = R3 Myelin Basic Protein
SIAEVLGLWRNV

SEQ ID NO: 319 - protein, 7 aa, synthetic construct
  REGION 1..7: note = R3 Myelin Basic Protein
SIRMTEI

SEQ ID NO: 320 - protein, 7 aa, synthetic construct
  REGION 1..7: note = R3 Myelin Basic Protein
SISKIRT

SEQ ID NO: 321 - protein, 7 aa, synthetic construct
  REGION 1..7: note = R3 Myelin Basic Protein
SLRSVGA

SEQ ID NO: 322 - protein, 7 aa, synthetic construct
  REGION 1..7: note = R3 Myelin Basic Protein
SSGMLSK

SEQ ID NO: 323 - protein, 7 aa, synthetic construct
  REGION 1..7: note = R3 Myelin Basic Protein
SYTLSRS

SEQ ID NO: 324 - protein, 7 aa, synthetic construct
  REGION 1..7: note = R3 Myelin Basic Protein
TLLTALT

SEQ ID NO: 325 - protein, 7 aa, synthetic construct
  REGION 1..7: note = R3 Myelin Basic Protein
TPLYLSS

SEQ ID NO: 326 - protein, 7 aa, synthetic construct
  REGION 1..7: note = R3 Myelin Basic Protein
VSFTLEP

SEQ ID NO: 327 - protein, 7 aa, synthetic construct
  REGION 1..7: note = R3 Myelin Basic Protein
VVDMSTY

SEQ ID NO: 328 - protein, 7 aa, synthetic construct
  REGION 1..7: note = R3 Myelin Basic Protein
WPDLRIL

SEQ ID NO: 329 - protein, 7 aa, synthetic construct
  REGION 1..7: note = R3 Myelin Basic Protein
WSLPLLS

SEQ ID NO: 330 - protein, 7 aa, synthetic construct
  REGION 1..7: note = R3 Myelin Basic Protein
WSPRWPS

SEQ ID NO: 331 - protein, 7 aa, synthetic construct
  REGION 1..7: note = R3 Myelin Basic Protein
YLPPPLP

SEQ ID NO: 332 - protein, 7 aa, synthetic construct
  REGION 1..7: note = R3 Myelin Basic Protein
DSYLLSA

SEQ ID NO: 333 - protein, 7 aa, synthetic construct
  REGION 1..7: note = R3 Myelin Basic Protein
FVLPNKN

SEQ ID NO: 334 - protein, 7 aa, synthetic construct
  REGION 1..7: note = R3 Myelin Basic Protein
IILPSAQ

SEQ ID NO: 335 - protein, 7 aa, synthetic construct
  REGION 1..7: note = R3 Myelin Basic Protein
KNSIAPR

SEQ ID NO: 336 - protein, 7 aa, synthetic construct
  REGION 1..7: note = R3 Myelin Basic Protein
LPPQISR

SEQ ID NO: 337 - protein, 7 aa, synthetic construct
  REGION 1..7: note = R3 Myelin Basic Protein
MPSLKHQ

SEQ ID NO: 338 - protein, 7 aa, synthetic construct
  REGION 1..7: note = R3 Myelin Basic Protein
NPTDTNK

SEQ ID NO: 339 - protein, 7 aa, synthetic construct
  REGION 1..7: note = R3 Myelin Basic Protein
NTSLSFK

SEQ ID NO: 340 - protein, 7 aa, synthetic construct
  REGION 1..7: note = R3 Myelin Basic Protein
QNSYLSN

SEQ ID NO: 341 - protein, 7 aa, synthetic construct
  REGION 1..7: note = R3 Myelin Basic Protein
QQAHLQS

SEQ ID NO: 342 - protein, 7 aa, synthetic construct
  REGION 1..7: note = R3 Myelin Basic Protein
SPPRFIP

SEQ ID NO: 343 - protein, 7 aa, synthetic construct
  REGION 1..7: note = R3 Myelin Basic Protein
SWFESHN

SEQ ID NO: 344 - protein, 7 aa, synthetic construct
  REGION 1..7: note = R3 Myelin Basic Protein
TPQVMLK

SEQ ID NO: 345 - protein, 7 aa, synthetic construct
  REGION 1..7: note = R3 Myelin Basic Protein
TSPPLAH

SEQ ID NO: 346 - protein, 7 aa, synthetic construct
  REGION 1..7: note = R3 Myelin Basic Protein
TSRLVST

SEQ ID NO: 347 - protein, 7 aa, synthetic construct
  REGION 1..7: note = R3 Myelin Basic Protein
VAHQRVS

SEQ ID NO: 348 - protein, 7 aa, synthetic construct
  REGION 1..7: note = R3 Myelin Basic Protein
VPTLRIP

SEQ ID NO: 349 - protein, 7 aa, synthetic construct
  REGION 1..7: note = R3 Myelin Basic Protein
YATATPS

SEQ ID NO: 350 - protein, 7 aa, synthetic construct
  REGION 1..7: note = R3 Myelin Basic Protein
ANLSRSV

SEQ ID NO: 351 - protein, 7 aa, synthetic construct
  REGION 1..7: note = R3 Myelin Basic Protein
FSARTNT

SEQ ID NO: 352 - protein, 7 aa, synthetic construct
  REGION 1..7: note = R3 Myelin Basic Protein
HFYGPGP

SEQ ID NO: 353 - protein, 7 aa, synthetic construct
  REGION 1..7: note = R3 Myelin Basic Protein
HPWLGNR

SEQ ID NO: 354 - protein, 7 aa, synthetic construct
  REGION 1..7: note = R3 Myelin Basic Protein
HYSPNVM

SEQ ID NO: 355 - protein, 7 aa, synthetic construct
  REGION 1..7: note = R3 Myelin Basic Protein
NFAQHMQ

SEQ ID NO: 356 - protein, 7 aa, synthetic construct
  REGION 1..7: note = R3 Myelin Basic Protein
SNAERWR

SEQ ID NO: 357 - protein, 7 aa, synthetic construct
  REGION 1..7: note = R3 Myelin Basic Protein
SRPTRVP

SEQ ID NO: 358 - protein, 7 aa, synthetic construct
  REGION 1..7: note = R3 Myelin Basic Protein
SVEYGQL

SEQ ID NO: 359 - protein, 7 aa, synthetic construct
  REGION 1..7: note = R3 Myelin Basic Protein
TGNLRLY

SEQ ID NO: 360 - protein, 7 aa, synthetic construct
  REGION 1..7: note = R3 Myelin Basic Protein
MDLSLKP

SEQ ID NO: 361 - protein, 7 aa, synthetic construct
  REGION 1..7: note = R3 Myelin Basic Protein
NTPKVLA

SEQ ID NO: 362 - protein, 7 aa, synthetic construct
  REGION 1..7: note = R3 Myelin Basic Protein
TQMTMDS

SEQ ID NO: 363 - protein, 7 aa, synthetic construct
  REGION 1..7: note = R3 Myelin PLP
ASLTLAL

SEQ ID NO: 364 - protein, 7 aa, synthetic construct
  REGION 1..7: note = R3 Myelin PLP
DRHEGHR

SEQ ID NO: 365 - protein, 7 aa, synthetic construct
  REGION 1..7: note = R3 Myelin PLP
FRIDKSM

SEQ ID NO: 366 - protein, 7 aa, synthetic construct
  REGION 1..7: note = R3 Myelin PLP
GHLQSLF

SEQ ID NO: 367 - protein, 7 aa, synthetic construct
  REGION 1..7: note = R3 Myelin PLP
GLWGPSL

SEQ ID NO: 368 - protein, 7 aa, synthetic construct
  REGION 1..7: note = R3 Myelin PLP
KPSASIY

SEQ ID NO: 369 - protein, 7 aa, synthetic construct
  REGION 1..7: note = R3 Myelin PLP
LPSHASI

SEQ ID NO: 370 - protein, 7 aa, synthetic construct
  REGION 1..7: note = R3 Myelin PLP
LPVKNLL

SEQ ID NO: 371 - protein, 7 aa, synthetic construct
  REGION 1..7: note = R3 Myelin PLP
MKIMPMD

SEQ ID NO: 372 - protein, 7 aa, synthetic construct
  REGION 1..7: note = R3 Myelin PLP
SAQSRSF

SEQ ID NO: 373 - protein, 7 aa, synthetic construct
  REGION 1..7: note = R3 Myelin PLP
SLPSLLP

SEQ ID NO: 374 - protein, 7 aa, synthetic construct
  REGION 1..7: note = R3 Myelin PLP
SVVYQNS

SEQ ID NO: 375 - protein, 7 aa, synthetic construct
  REGION 1..7: note = R3 Myelin PLP
TTTIADM

SEQ ID NO: 376 - protein, 7 aa, synthetic construct
  REGION 1..7: note = R3 Myelin PLP
VTGPGTP

SEQ ID NO: 377 - protein, 7 aa, synthetic construct
  REGION 1..7: note = R3 Myelin PLP
VTPNKAR

SEQ ID NO: 378 - protein, 7 aa, synthetic construct
  REGION 1..7: note = R3 Myelin PLP
WHYTPSM

SEQ ID NO: 379 - protein, 7 aa, synthetic construct
  REGION 1..7: note = R3 Myelin PLP
WSRISVD

SEQ ID NO: 380 - protein, 7 aa, synthetic construct
  REGION 1..7: note = R3 Myelin PLP
ALFQERK

SEQ ID NO: 381 - protein, 7 aa, synthetic construct
  REGION 1..7: note = R3 Myelin PLP
AQASSAR

SEQ ID NO: 382 - protein, 7 aa, synthetic construct
  REGION 1..7: note = R3 Myelin PLP
EVLYPNN

SEQ ID NO: 383 - protein, 7 aa, synthetic construct
  REGION 1..7: note = R3 Myelin PLP
HLHTRPT

SEQ ID NO: 384 - protein, 7 aa, synthetic construct
  REGION 1..7: note = R3 Myelin PLP
IRPTHNG

SEQ ID NO: 385 - protein, 7 aa, synthetic construct
  REGION 1..7: note = R3 Myelin PLP
KGVIPAT

SEQ ID NO: 386 - protein, 7 aa, synthetic construct
  REGION 1..7: note = R3 Myelin PLP
LHQTYRP

SEQ ID NO: 387 - protein, 7 aa, synthetic construct
  REGION 1..7: note = R3 Myelin PLP
LPHRLNP

SEQ ID NO: 388 - protein, 7 aa, synthetic construct
  REGION 1..7: note = R3 Myelin PLP
TGISSTP

SEQ ID NO: 389 - protein, 7 aa, synthetic construct
  REGION 1..7: note = R3 Myelin PLP
TMPIKAM

SEQ ID NO: 390 - protein, 7 aa, synthetic construct
  REGION 1..7: note = R3 Myelin PLP
VPGHISG

SEQ ID NO: 391 - protein, 7 aa, synthetic construct
  REGION 1..7: note = R3 Myelin PLP
VSASWMP

SEQ ID NO: 392 - protein, 7 aa, synthetic construct
  REGION 1..7: note = R3 Myelin PLP
WSPHGYK

SEQ ID NO: 393 - protein, 7 aa, synthetic construct
  REGION 1..7: note = R3 Myelin PLP
YSQAISA

SEQ ID NO: 394 - protein, 7 aa, synthetic construct
  REGION 1..7: note = R3 Myelin PLP
AVVAMNK

SEQ ID NO: 395 - protein, 7 aa, synthetic construct
  REGION 1..7: note = R3 Myelin PLP
GIPSSKN

SEQ ID NO: 396 - protein, 7 aa, synthetic construct
  REGION 1..7: note = R3 Myelin PLP
NDASTVS

SEQ ID NO: 397 - protein, 7 aa, synthetic construct
  REGION 1..7: note = R3 Myelin PLP
QLPRNNL

SEQ ID NO: 398 - protein, 7 aa, synthetic construct
  REGION 1..7: note = R3 Myelin PLP
SLNRGGA

SEQ ID NO: 399 - protein, 7 aa, synthetic construct
  REGION 1..7: note = R3 Myelin PLP
SLPGYRH

SEQ ID NO: 400 - protein, 7 aa, synthetic construct
  REGION 1..7: note = R3 Myelin PLP
SVLPDKL

SEQ ID NO: 401 - protein, 7 aa, synthetic construct
  REGION 1..7: note = R3 Myelin PLP
TLWAQKT

SEQ ID NO: 402 - protein, 7 aa, synthetic construct
  REGION 1..7: note = R3 Myelin PLP
TQPRYPS

SEQ ID NO: 403 - protein, 7 aa, synthetic construct
  REGION 1..7: note = R3 Myelin PLP
VFPERRV

SEQ ID NO: 404 - protein, 7 aa, synthetic construct
  REGION 1..7: note = R3 Myelin PLP
VGYRSAS

SEQ ID NO: 405 - protein, 7 aa, synthetic construct
  REGION 1..7: note = R3 Myelin PLP
VRTSMNH

SEQ ID NO: 406 - protein, 7 aa, synthetic construct
  REGION 1..7: note = R3 Myelin PLP
YTWTPSR

SEQ ID NO: 407 - protein, 7 aa, synthetic construct
  REGION 1..7: note = R3 Myelin PLP
FSLDRDG

SEQ ID NO: 408 - protein, 7 aa, synthetic construct
  REGION 1..7: note = R3 Myelin PLP
FSTPTNV

SEQ ID NO: 409 - protein, 7 aa, synthetic construct
  REGION 1..7: note = R3 Myelin PLP
GPAAVII

SEQ ID NO: 410 - protein, 7 aa, synthetic construct
  REGION 1..7: note = R3 Myelin PLP
GWSTAIR

SEQ ID NO: 411 - protein, 7 aa, synthetic construct
  REGION 1..7: note = R3 Myelin PLP
HKAPLGT

SEQ ID NO: 412 - protein, 7 aa, synthetic construct
  REGION 1..7: note = R3 Myelin PLP
IDRVRGL

SEQ ID NO: 413 - protein, 7 aa, synthetic construct
  REGION 1..7: note = R3 Myelin PLP
LMQKPSI

SEQ ID NO: 414 - protein, 7 aa, synthetic construct
  REGION 1..7: note = R3 Myelin PLP
SVMWMTP

SEQ ID NO: 415 - protein, 7 aa, synthetic construct
  REGION 1..7: note = R3 Myelin PLP
TKITPHR

SEQ ID NO: 416 - protein, 7 aa, synthetic construct
  REGION 1..7: note = R3 Myelin PLP
YSSRLTA

SEQ ID NO: 417 - protein, 7 aa, synthetic construct
  REGION 1..7: note = R3 Myelin PLP
ARFVPLT

SEQ ID NO: 418 - protein, 7 aa, synthetic construct
  REGION 1..7: note = R3 Myelin PLP
DGSRDLV

SEQ ID NO: 419 - protein, 7 aa, synthetic construct
  REGION 1..7: note = R3 Myelin PLP
EKSTTVA

SEQ ID NO: 420 - protein, 7 aa, synthetic construct
  REGION 1..7: note = R3 Myelin PLP
YSDTYKH

SEQ ID NO: 421 - protein, 7 aa, synthetic construct
  REGION 1..7: note = R3 Myelin PLP
EPSSFTF

SEQ ID NO: 422 - protein, 7 aa, synthetic construct
  REGION 1..7: note = R3 Myelin PLP
GKSYSQI

SEQ ID NO: 423 - protein, 7 aa, synthetic construct
  REGION 1..7: note = R3 Myelin PLP
GNVFQTS

SEQ ID NO: 424 - protein, 7 aa, synthetic construct
  REGION 1..7: note = R3 Myelin PLP
IIPPTSV

SEQ ID NO: 425 - protein, 7 aa, synthetic construct
  REGION 1..7: note = R3 Myelin PLP
LPLRLHA

SEQ ID NO: 426 - protein, 7 aa, synthetic construct
  REGION 1..7: note = R3 Myelin PLP
NVFSATP

SEQ ID NO: 427 - protein, 7 aa, synthetic construct
  REGION 1..7: note = R3 Myelin PLP
QRRSVIL

SEQ ID NO: 428 - protein, 7 aa, synthetic construct
  REGION 1..7: note = R3 Myelin PLP
SGVATYT

SEQ ID NO: 429 - protein, 7 aa, synthetic construct
  REGION 1..7: note = R3 Myelin PLP
SWDVLEL

SEQ ID NO: 430 - protein, 7 aa, synthetic construct
  REGION 1..7: note = R3 Myelin PLP
TFPERLR

SEQ ID NO: 431 - protein, 7 aa, synthetic construct
  REGION 1..7: note = R3 Myelin PLP
WSPHRSF

SEQ ID NO: 432 - protein, 7 aa, synthetic construct
  REGION 1..7: note = R3 Myelin PLP
IHHLESS

SEQ ID NO: 433 - protein, 7 aa, synthetic construct
  REGION 1..7: note = R3 Myelin PLP
LNGISAL

SEQ ID NO: 434 - protein, 7 aa, synthetic construct
  REGION 1..7: note = R3 Myelin PLP
SALISTR

SEQ ID NO: 435 - protein, 7 aa, synthetic construct
  REGION 1..7: note = R3 Myelin PLP
SGLHYAL

SEQ ID NO: 436 - protein, 7 aa, synthetic construct
  REGION 1..7: note = R3 Myelin PLP
TRASYPQ

SEQ ID NO: 437 - protein, 7 aa, synthetic construct
  REGION 1..7: note = R3 Myelin PLP
NIPSSIL

SEQ ID NO: 438 - protein, 7 aa, synthetic construct
  REGION 1..7: note = R3 Myelin PLP
QPNMLKP

SEQ ID NO: 439 - protein, 7 aa, synthetic construct
  REGION 1..7: note = R3 Myelin PLP
RLQPEPT

SEQ ID NO: 440 - protein, 7 aa, synthetic construct
  REGION 1..7: note = R3 Myelin PLP
SVEKYSK

SEQ ID NO: 441 - protein, 7 aa, synthetic construct
  REGION 1..7: note = R3 Myelin PLP
VSSQEQA

SEQ ID NO: 442 - protein, 7 aa, synthetic construct
  REGION 1..7: note = R3 Myelin PLP
WNTADRL

SEQ ID NO: 443 - protein, 7 aa, synthetic construct
  REGION 1..7: note = R3 Myelin PLP
MPHAPVQ

SEQ ID NO: 444 - protein, 7 aa, synthetic construct
  REGION 1..7: note = R3 Myelin PLP
TDVEMVP

SEQ ID NO: 445 - protein, 7 aa, synthetic construct
  REGION 1..7: note = R3 Myelin PLP
AHQSSVT

SEQ ID NO: 446 - protein, 7 aa, synthetic construct
  REGION 1..7: note = R3 Myelin PLP
GSAWKKH

SEQ ID NO: 447 - protein, 9 aa, synthetic construct
  REGION 1..9: note = R3 Nidogen-2
CIHHKGVLC

SEQ ID NO: 448 - protein, 9 aa, synthetic construct
  REGION 1..9: note = R3 Nidogen-2
CKDGHIRHC

SEQ ID NO: 449 - protein, 9 aa, synthetic construct
  REGION 1..9: note = R3 Nidogen-2
CNLATSLQC

SEQ ID NO: 450 - protein, 9 aa, synthetic construct
  REGION 1..9: note = R3 Nidogen-2
CRSPHEPMC

SEQ ID NO: 451 - protein, 9 aa, synthetic construct
  REGION 1..9: note = R3 Nidogen-2
CVDKMASVC

SEQ ID NO: 452 - protein, 9 aa, synthetic construct
  REGION 1..9: note = R3 Nidogen-2
CYSAPTKSC

SEQ ID NO: 453 - protein, 7 aa, synthetic construct
  REGION 1..7: note = R3 Nidogen-2
EAMMHRN

SEQ ID NO: 454 - protein, 7 aa, synthetic construct
  REGION 1..7: note = R3 Nidogen-2
LHITPEV

SEQ ID NO: 455 - protein, 7 aa, synthetic construct
  REGION 1..7: note = R3 Nidogen-2
LSIRGLT

SEQ ID NO: 456 - protein, 7 aa, synthetic construct
  REGION 1..7: note = R3 Nidogen-2
NILSQVN

SEQ ID NO: 457 - protein, 7 aa, synthetic construct
  REGION 1..7: note = R3 Nidogen-2
QLLESRT

SEQ ID NO: 458 - protein, 7 aa, synthetic construct
  REGION 1..7: note = R3 Nidogen-2
QMDAKHM

SEQ ID NO: 459 - protein, 7 aa, synthetic construct
  REGION 1..7: note = R3 Nidogen-2
QTLRPKQ

SEQ ID NO: 460 - protein, 7 aa, synthetic construct
  REGION 1..7: note = R3 Nidogen-2
TIASLLV

SEQ ID NO: 461 - protein, 7 aa, synthetic construct
  REGION 1..7: note = R3 Nidogen-2
WLAAGSQ

SEQ ID NO: 462 - protein, 7 aa, synthetic construct
  REGION 1..7: note = R3 Nidogen-2
ALYLPGR

SEQ ID NO: 463 - protein, 7 aa, synthetic construct
  REGION 1..7: note = R3 Nidogen-2
ARMAFSL

SEQ ID NO: 464 - protein, 7 aa, synthetic construct
  REGION 1..7: note = R3 Nidogen-2
ATYNMSQ

SEQ ID NO: 465 - protein, 7 aa, synthetic construct
  REGION 1..7: note = R3 Nidogen-2
GLRTMEP

SEQ ID NO: 466 - protein, 7 aa, synthetic construct
  REGION 1..7: note = R3 Nidogen-2
TPWLPTI

SEQ ID NO: 467 - protein, 7 aa, synthetic construct
  REGION 1..7: note = R3 Nidogen-2
YNHTMMY

SEQ ID NO: 468 - protein, 9 aa, synthetic construct
  REGION 1..9: note = R3 Nidogen-2
CSFKMNQKC

SEQ ID NO: 469 - protein, 7 aa, synthetic construct
  REGION 1..7: note = R3 Nidogen-2
LVHPFHG

SEQ ID NO: 470 - protein, 9 aa, synthetic construct
  REGION 1..9: note = R3 Nidogen-2
CSSSAPRIC

SEQ ID NO: 471 - protein, 7 aa, synthetic construct
  REGION 1..7: note = R3 Nidogen-2
HSRLPTP

SEQ ID NO: 472 - protein, 9 aa, synthetic construct
  REGION 1..9: note = Laminin 421/521
CKSPIKGTC

SEQ ID NO: 473 - protein, 9 aa, synthetic construct
  REGION 1..9: note = Laminin 421/521
CLQKNHKFC

SEQ ID NO: 474 - protein, 12 aa, synthetic construct
  REGION 1..12: note = Laminin 421/521
CVLKCEDQKYRG

SEQ ID NO: 475 - protein, 12 aa, synthetic construct
  REGION 1..12: note = Laminin 421/521
DGANMFNIAPAN

SEQ ID NO: 476 - protein, 12 aa, synthetic construct
  REGION 1..12: note = Laminin 421/521
DRMYSTVPAEGL

SEQ ID NO: 477 - protein, 7 aa, synthetic construct
  REGION 1..7: note = Laminin 421/521
EAVSTQI

SEQ ID NO: 478 - protein, 12 aa, synthetic construct
  REGION 1..12: note = Laminin 421/521
GINLRALDLHAN

SEQ ID NO: 479 - protein, 12 aa, synthetic construct
  REGION 1..12: note = Laminin 421/521
HQSVTGVRSHFH

SEQ ID NO: 480 - protein, 12 aa, synthetic construct
  REGION 1..12: note = Laminin 421/521
SVVGWAAPRTAQ

SEQ ID NO: 481 - protein, 12 aa, synthetic construct
  REGION 1..12: note = Laminin 421/521
TILKPAAQGFAD

SEQ ID NO: 482 - protein, 7 aa, synthetic construct
  REGION 1..7: note = Laminin 421/521
TSAVSLR

SEQ ID NO: 483 - protein, 12 aa, synthetic construct
  REGION 1..12: note = Laminin 421/521
TTLWLDRDEALK

SEQ ID NO: 484 - protein, 12 aa, synthetic construct
  REGION 1..12: note = Laminin 421/521
VIADQSKSAVAV

SEQ ID NO: 485 - protein, 12 aa, synthetic construct
  REGION 1..12: note = Laminin 421/521
AQNLRVHAWASL

SEQ ID NO: 486 - protein, 9 aa, synthetic construct
  REGION 1..9: note = Laminin 421/521
CPMHQSKTC

SEQ ID NO: 487 - protein, 9 aa, synthetic construct
  REGION 1..9: note = Laminin 421/521
CTDGRNFVC

SEQ ID NO: 488 - protein, 9 aa, synthetic construct
  REGION 1..9: note = Laminin 421/521
CTGETLLTC

SEQ ID NO: 489 - protein, 9 aa, synthetic construct
  REGION 1..9: note = Laminin 421/521
CTSSSHQTC

SEQ ID NO: 490 - protein, 12 aa, synthetic construct
  REGION 1..12: note = Laminin 421/521
ETGGLAYGSGQK

SEQ ID NO: 491 - protein, 12 aa, synthetic construct
  REGION 1..12: note = Laminin 421/521
GIVPSRHTTGLG

SEQ ID NO: 492 - protein, 12 aa, synthetic construct
  REGION 1..12: note = Laminin 421/521
GWTSDLSRNVRG

SEQ ID NO: 493 - protein, 7 aa, synthetic construct
  REGION 1..7: note = Laminin 421/521
HTQIHRL

SEQ ID NO: 494 - protein, 12 aa, synthetic construct
  REGION 1..12: note = Laminin 421/521
KFADTKLTSLRY

SEQ ID NO: 495 - protein, 7 aa, synthetic construct
  REGION 1..7: note = Laminin 421/521
QLQHVHL

SEQ ID NO: 496 - protein, 12 aa, synthetic construct
  REGION 1..12: note = Laminin 421/521
QPTPSQIKFTRT

SEQ ID NO: 497 - protein, 12 aa, synthetic construct
  REGION 1..12: note = Laminin 421/521
RALDRYLPWSPH

SEQ ID NO: 498 - protein, 12 aa, synthetic construct
  REGION 1..12: note = Laminin 421/521
SLPYAASLNSVE

SEQ ID NO: 499 - protein, 7 aa, synthetic construct
  REGION 1..7: note = Laminin 421/521
STNVQRA

SEQ ID NO: 500 - protein, 12 aa, synthetic construct
  REGION 1..12: note = Laminin 421/521
SVLSTGTANQRH

SEQ ID NO: 501 - protein, 12 aa, synthetic construct
  REGION 1..12: note = Laminin 421/521
SVQTPMWRSLVG

SEQ ID NO: 502 - protein, 12 aa, synthetic construct
  REGION 1..12: note = Laminin 421/521
SWHFTGTPFMNR

SEQ ID NO: 503 - protein, 12 aa, synthetic construct
  REGION 1..12: note = Laminin 421/521
SYTATWSEMSRS

SEQ ID NO: 504 - protein, 12 aa, synthetic construct
  REGION 1..12: note = Laminin 421/521
TALSHRHEAMRW

SEQ ID NO: 505 - protein, 7 aa, synthetic construct
  REGION 1..7: note = Laminin 421/521
TKGIAPK

SEQ ID NO: 506 - protein, 12 aa, synthetic construct
  REGION 1..12: note = Laminin 421/521
TVARSTAQERSI

SEQ ID NO: 507 - protein, 12 aa, synthetic construct
  REGION 1..12: note = Laminin 421/521
AVRPLGLPDNHR

SEQ ID NO: 508 - protein, 9 aa, synthetic construct
  REGION 1..9: note = Laminin 421/521
CIGGPHRNC

SEQ ID NO: 509 - protein, 9 aa, synthetic construct
  REGION 1..9: note = Laminin 421/521
CKSPAIKGC

SEQ ID NO: 510 - protein, 9 aa, synthetic construct
  REGION 1..9: note = Laminin 421/521
CNSWKAAKC

SEQ ID NO: 511 - protein, 12 aa, synthetic construct
  REGION 1..12: note = Laminin 421/521
DSLYRGMHQPRI

SEQ ID NO: 512 - protein, 12 aa, synthetic construct
  REGION 1..12: note = Laminin 421/521
EDRMMTYRYTST

SEQ ID NO: 513 - protein, 12 aa, synthetic construct
  REGION 1..12: note = Laminin 421/521
EVERILTPHVNN

SEQ ID NO: 514 - protein, 12 aa, synthetic construct
  REGION 1..12: note = Laminin 421/521
FNSDSRSTHQED

SEQ ID NO: 515 - protein, 12 aa, synthetic construct
  REGION 1..12: note = Laminin 421/521
HPAWADFFTMSS

SEQ ID NO: 516 - protein, 7 aa, synthetic construct
  REGION 1..7: note = Laminin 421/521
IDLSLRS

SEQ ID NO: 517 - protein, 12 aa, synthetic construct
  REGION 1..12: note = Laminin 421/521
IDQSGLQKSGMK

SEQ ID NO: 518 - protein, 12 aa, synthetic construct
  REGION 1..12: note = Laminin 421/521
LGSSRSPSSFLG

SEQ ID NO: 519 - protein, 12 aa, synthetic construct
  REGION 1..12: note = Laminin 421/521
NGHEVQSRAANR

SEQ ID NO: 520 - protein, 12 aa, synthetic construct
  REGION 1..12: note = Laminin 421/521
QGWKDRLPIWRY

SEQ ID NO: 521 - protein, 12 aa, synthetic construct
  REGION 1..12: note = Laminin 421/521
SHDVANGTSVRT

SEQ ID NO: 522 - protein, 12 aa, synthetic construct
  REGION 1..12: note = Laminin 421/521
SPDRTNWGWQTN

SEQ ID NO: 523 - protein, 12 aa, synthetic construct
  REGION 1..12: note = Laminin 421/521
SVESNSKLTMPR

SEQ ID NO: 524 - protein, 7 aa, synthetic construct
  REGION 1..7: note = Laminin 421/521
WPMMQTR

SEQ ID NO: 525 - protein, 9 aa, synthetic construct
  REGION 1..9: note = Laminin 421/521
CKSERGPEC

SEQ ID NO: 526 - protein, 9 aa, synthetic construct
  REGION 1..9: note = Laminin 421/521
CQGWPRPMC

SEQ ID NO: 527 - protein, 9 aa, synthetic construct
  REGION 1..9: note = Laminin 421/521
CSRAGLSAC

SEQ ID NO: 528 - protein, 9 aa, synthetic construct
  REGION 1..9: note = Laminin 421/521
CTWKHRDNC

SEQ ID NO: 529 - protein, 12 aa, synthetic construct
  REGION 1..12: note = Laminin 421/521
GLLDWGSLQGGN

SEQ ID NO: 530 - protein, 7 aa, synthetic construct
  REGION 1..7: note = Laminin 421/521
GPLLFRG

SEQ ID NO: 531 - protein, 7 aa, synthetic construct
  REGION 1..7: note = Laminin 421/521
GSPEKRT

SEQ ID NO: 532 - protein, 7 aa, synthetic construct
  REGION 1..7: note = Laminin 421/521
KASIAYD

SEQ ID NO: 533 - protein, 12 aa, synthetic construct
  REGION 1..12: note = Laminin 421/521
LGYGQGTPHRSN

SEQ ID NO: 534 - protein, 12 aa, synthetic construct
  REGION 1..12: note = Laminin 421/521
QGWEFQVPARHS

SEQ ID NO: 535 - protein, 12 aa, synthetic construct
  REGION 1..12: note = Laminin 421/521
QTFGYSMFNVRT

SEQ ID NO: 536 - protein, 12 aa, synthetic construct
  REGION 1..12: note = Laminin 421/521
RNLHYPLNHPFM

SEQ ID NO: 537 - protein, 7 aa, synthetic construct
  REGION 1..7: note = Laminin 421/521
TAPRWTQ

SEQ ID NO: 538 - protein, 7 aa, synthetic construct
  REGION 1..7: note = Laminin 421/521
VPSYILR

SEQ ID NO: 539 - protein, 7 aa, synthetic construct
  REGION 1..7: note = Laminin 421/521
WRSPLTT

SEQ ID NO: 540 - protein, 9 aa, synthetic construct
  REGION 1..9: note = Laminin 421/521
CQTSGHHQC

SEQ ID NO: 541 - protein, 9 aa, synthetic construct
  REGION 1..9: note = Laminin 421/521
CSSQAKRSC

SEQ ID NO: 542 - protein, 9 aa, synthetic construct
  REGION 1..9: note = Laminin 421/521
CTAWTRQEC

SEQ ID NO: 543 - protein, 12 aa, synthetic construct
  REGION 1..12: note = Laminin 421/521
EYVKFSHSPRTY

SEQ ID NO: 544 - protein, 12 aa, synthetic construct
  REGION 1..12: note = Laminin 421/521
GGWQSLWDKPEH

SEQ ID NO: 545 - protein, 12 aa, synthetic construct
  REGION 1..12: note = Laminin 421/521
GPMLKNLSDAVT

SEQ ID NO: 546 - protein, 12 aa, synthetic construct
  REGION 1..12: note = Laminin 421/521
NHDQGSLTRWRS

SEQ ID NO: 547 - protein, 12 aa, synthetic construct
  REGION 1..12: note = Laminin 421/521
SLLSVVNTSSKS

SEQ ID NO: 548 - protein, 12 aa, synthetic construct
  REGION 1..12: note = Laminin 421/521
VVPSWPSVHRPP

SEQ ID NO: 549 - protein, 12 aa, synthetic construct
  REGION 1..12: note = Laminin 421/521
WGWNGANMSPRG

SEQ ID NO: 550 - protein, 9 aa, synthetic construct
  REGION 1..9: note = Laminin 421/521
CHNADNNGC

SEQ ID NO: 551 - protein, 7 aa, synthetic construct
  REGION 1..7: note = Laminin 421/521
LPMLRHS

SEQ ID NO: 552 - protein, 12 aa, synthetic construct
  REGION 1..12: note = Laminin 421/521
NDTGRHASGISK

SEQ ID NO: 553 - protein, 12 aa, synthetic construct
  REGION 1..12: note = Laminin 421/521
QALRTNYSPLNS

SEQ ID NO: 554 - protein, 12 aa, synthetic construct
  REGION 1..12: note = Laminin 421/521
TLKAGTRANDGV

SEQ ID NO: 555 - protein, 9 aa, synthetic construct
  REGION 1..9: note = Laminin 421/521
CGETTAGKC

SEQ ID NO: 556 - protein, 7 aa, synthetic construct
  REGION 1..7: note = Laminin 421/521
HSTYAPG

SEQ ID NO: 557 - protein, 12 aa, synthetic construct
  REGION 1..12: note = Laminin 421/521
QINWKQADKNAD

SEQ ID NO: 558 - protein, 12 aa, synthetic construct
  REGION 1..12: note = Laminin 421/521
ALPWQSGVHGTK

SEQ ID NO: 559 - protein, 12 aa, synthetic construct
  REGION 1..12: note = Laminin 421/521
TFGSGRSMPIQY

SEQ ID NO: 560 - protein, 12 aa, synthetic construct
  REGION 1..12: note = Laminin 421/521
VYRNGGGLPLTA

SEQ ID NO: 561 - protein, 12 aa, synthetic construct
  REGION 1..12: note = Laminin 421/521
ALKIGPETTIYM

SEQ ID NO: 562 - protein, 12 aa, synthetic construct
  REGION 1..12: note = Laminin 421/521
DWTQVRVTNWFL

SEQ ID NO: 563 - protein, 12 aa, synthetic construct
  REGION 1..12: note = Laminin 421/521
LGHQNGGRADMW

SEQ ID NO: 564 - protein, 7 aa, synthetic construct
  REGION 1..7: note = Laminin 421/521
MTNSGAK

SEQ ID NO: 565 - protein, 7 aa, synthetic construct
  REGION 1..7: note = Laminin 421/521
WAYTDYM

SEQ ID NO: 566 - protein, 12 aa, synthetic construct
  REGION 1..12: note = Laminin 421/521
AHAAGRDMRQGT

SEQ ID NO: 567 - protein, 12 aa, synthetic construct
  REGION 1..12: note = Laminin 421/521
DVIHSSRGAYFE

SEQ ID NO: 568 - protein, 12 aa, synthetic construct
  REGION 1..12: note = Laminin 421/521
NWTHLGVARLQP

SEQ ID NO: 569 - protein, 12 aa, synthetic construct
  REGION 1..12: note = Laminin 421/521
SYDGTMLKQVRL

SEQ ID NO: 570 - protein, 12 aa, synthetic construct
  REGION 1..12: note = Laminin 421/521
WQWRSTELGYRY

SEQ ID NO: 571 - protein, 12 aa, synthetic construct
  REGION 1..12: note = Laminin 421/521
WSPANGWRHQTI

SEQ ID NO: 572 - protein, 9 aa, synthetic construct
  REGION 1..9: note = Laminin 421/521
CLHNGQRSC

SEQ ID NO: 573 - protein, 9 aa, synthetic construct
  REGION 1..9: note = Laminin 421/521
CTPRSATLC

SEQ ID NO: 574 - protein, 12 aa, synthetic construct
  REGION 1..12: note = Laminin 421/521
DIMKSPRSNLRS

SEQ ID NO: 575 - protein, 12 aa, synthetic construct
  REGION 1..12: note = Laminin 421/521
QTFHEYLNPARG

SEQ ID NO: 576 - protein, 12 aa, synthetic construct
  REGION 1..12: note = Laminin 421/521
SLNTTWVSPMMK

SEQ ID NO: 577 - protein, 7 aa, synthetic construct
  REGION 1..7: note = Laminin 421/521
AQRMNQA

SEQ ID NO: 578 - protein, 7 aa, synthetic construct
  REGION 1..7: note = Laminin 421/521
GDAQILM

SEQ ID NO: 579 - protein, 12 aa, synthetic construct
  REGION 1..12: note = Laminin 421/521
GHRTLVTSERYL

SEQ ID NO: 580 - protein, 12 aa, synthetic construct
  REGION 1..12: note = Laminin 421/521
LGPSTQGAGQTR

SEQ ID NO: 581 - protein, 12 aa, synthetic construct
  REGION 1..12: note = Laminin 421/521
TFAAAQAELIMV

SEQ ID NO: 582 - protein, 12 aa, synthetic construct
  REGION 1..12: note = Laminin 421/521
WEEHRVEILPDV

SEQ ID NO: 583 - protein, 12 aa, synthetic construct
  REGION 1..12: note = Laminin 421/521
AGAYTSRHAFDE

SEQ ID NO: 584 - protein, 9 aa, synthetic construct
  REGION 1..9: note = Laminin 421/521
CGLATNKSC

SEQ ID NO: 585 - protein, 12 aa, synthetic construct
  REGION 1..12: note = Laminin 421/521
GDVITAYINPWP

SEQ ID NO: 586 - protein, 12 aa, synthetic construct
  REGION 1..12: note = Laminin 421/521
GYDLSRLWGMAS

SEQ ID NO: 587 - protein, 7 aa, synthetic construct
  REGION 1..7: note = Laminin 421/521
ANRYLAS

SEQ ID NO: 588 - protein, 9 aa, synthetic construct
  REGION 1..9: note = Laminin 421/521
CKWTQLWGC

SEQ ID NO: 589 - protein, 9 aa, synthetic construct
  REGION 1..9: note = Laminin 421/521
CLKDTHLNC

SEQ ID NO: 590 - protein, 12 aa, synthetic construct
  REGION 1..12: note = Laminin 421/521
GQPRNIHLPGTH

SEQ ID NO: 591 - protein, 12 aa, synthetic construct
  REGION 1..12: note = Laminin 421/521
GSTTHPHFGLPG

SEQ ID NO: 592 - protein, 12 aa, synthetic construct
  REGION 1..12: note = Laminin 421/521
GWIQDTFVLGRS

SEQ ID NO: 593 - protein, 7 aa, synthetic construct
  REGION 1..7: note = Laminin 421/521
QGHPFIY

SEQ ID NO: 594 - protein, 12 aa, synthetic construct
  REGION 1..12: note = Laminin 421/521
SNPKHVSSLGQM

SEQ ID NO: 595 - protein, 12 aa, synthetic construct
  REGION 1..12: note = Laminin 421/521
WGANTTGRTHGG

SEQ ID NO: 596 - protein, 12 aa, synthetic construct
  REGION 1..12: note = Laminin 421/521
AQGVWWSEWFAP

SEQ ID NO: 597 - protein, 9 aa, synthetic construct
  REGION 1..9: note = Laminin 421/521
CFQPKMNSC

SEQ ID NO: 598 - protein, 9 aa, synthetic construct
  REGION 1..9: note = Laminin 421/521
CWSGNSRSC

SEQ ID NO: 599 - protein, 7 aa, synthetic construct
  REGION 1..7: note = Laminin 421/521
FPLKIRT

SEQ ID NO: 600 - protein, 12 aa, synthetic construct
  REGION 1..12: note = Laminin 421/521
LARSSIMAANNV

SEQ ID NO: 601 - protein, 12 aa, synthetic construct
  REGION 1..12: note = Laminin 421/521
AKTQAPSNWSGV

SEQ ID NO: 602 - protein, 9 aa, synthetic construct
  REGION 1..9: note = Laminin 421/521
CSVWNSGNC

SEQ ID NO: 603 - protein, 9 aa, synthetic construct
  REGION 1..9: note = Laminin 421/521
CTGRISKHC

SEQ ID NO: 604 - protein, 12 aa, synthetic construct
  REGION 1..12: note = Laminin 421/521
GAMMNSNNLVAR

SEQ ID NO: 605 - protein, 12 aa, synthetic construct
  REGION 1..12: note = Laminin 421/521
GGGYHIFGPLVT

SEQ ID NO: 606 - protein, 7 aa, synthetic construct
  REGION 1..7: note = Laminin 421/521
HSFLGAR

SEQ ID NO: 607 - protein, 7 aa, synthetic construct
  REGION 1..7: note = Laminin 421/521
LLAGPFR

SEQ ID NO: 608 - protein, 7 aa, synthetic construct
  REGION 1..7: note = Laminin 421/521
QTLQTRH

SEQ ID NO: 609 - protein, 12 aa, synthetic construct
  REGION 1..12: note = Laminin 421/521
ALGGEPRKAYQR

SEQ ID NO: 610 - protein, 9 aa, synthetic construct
  REGION 1..9: note = Laminin 421/521
CNHLTQKLC

SEQ ID NO: 611 - protein, 9 aa, synthetic construct
  REGION 1..9: note = Laminin 421/521
CTSKLARHC

SEQ ID NO: 612 - protein, 12 aa, synthetic construct
  REGION 1..12: note = Laminin 421/521
AVYPLDLGAGMR

SEQ ID NO: 613 - protein, 9 aa, synthetic construct
  REGION 1..9: note = Laminin 421/521
CHRTYNSTC

SEQ ID NO: 614 - protein, 12 aa, synthetic construct
  REGION 1..12: note = Laminin 421/521
LDRNADSVRAVL

SEQ ID NO: 615 - protein, 12 aa, synthetic construct
  REGION 1..12: note = Laminin 421/521
TTNGVPGHDRSP

SEQ ID NO: 616 - protein, 12 aa, synthetic construct
  REGION 1..12: note = Laminin 421/521
ADMPQMTLKYGV

SEQ ID NO: 617 - protein, 7 aa, synthetic construct
  REGION 1..7: note = Laminin 421/521
AWNGNRS

SEQ ID NO: 618 - protein, 7 aa, synthetic construct
  REGION 1..7: note = Laminin 421/521
GDLLLKR

SEQ ID NO: 619 - protein, 7 aa, synthetic construct
  REGION 1..7: note = Laminin 421/521
VPQIRIK

SEQ ID NO: 620 - protein, 7 aa, synthetic construct
  REGION 1..7: note = Laminin 421/521
SWQMRSY

SEQ ID NO: 621 - protein, 12 aa, synthetic construct
  REGION 1..12: note = Laminin 421/521
YHLGPNQKMRTS

SEQ ID NO: 622 - protein, 12 aa, synthetic construct
  REGION 1..12: note = Laminin 421/521
ASHSRPLMNYAP

SEQ ID NO: 623 - protein, 12 aa, synthetic construct
  REGION 1..12: note = Laminin 421/521
QTRWDDGSYQIS

SEQ ID NO: 624 - protein, 12 aa, synthetic construct
  REGION 1..12: note = Laminin 421/521
SLAVANTRFMIR

SEQ ID NO: 625 - protein, 12 aa, synthetic construct
  REGION 1..12: note = Laminin 421/521
EGHVWSEYTWGT

SEQ ID NO: 626 - protein, 12 aa, synthetic construct
  REGION 1..12: note = Laminin 421/521
ETYKVTRVISPW

SEQ ID NO: 627 - protein, 12 aa, synthetic construct
  REGION 1..12: note = Laminin 421/521
GHPWTEIDFMSS

SEQ ID NO: 628 - protein, 12 aa, synthetic construct
  REGION 1..12: note = Laminin 421/521
KQELSDNLASHR

SEQ ID NO: 629 - protein, 12 aa, synthetic construct
  REGION 1..12: note = Laminin 421/521
SFRNLEKLSLWS

SEQ ID NO: 630 - protein, 7 aa, synthetic construct
  REGION 1..7: note = Laminin 421/521
GDLWNVP

SEQ ID NO: 631 - protein, 7 aa, synthetic construct
  REGION 1..7: note = Laminin 421/521
GPKNHHQ

SEQ ID NO: 632 - protein, 12 aa, synthetic construct
  REGION 1..12: note = Laminin 421/521
GSGFRNEEHSAH

SEQ ID NO: 633 - protein, 12 aa, synthetic construct
  REGION 1..12: note = Laminin 421/521
NNNAAMQNHGVR

SEQ ID NO: 634 - protein, 7 aa, synthetic construct
  REGION 1..7: note = Laminin 421/521
RAQAHQV

SEQ ID NO: 635 - protein, 9 aa, synthetic construct
  REGION 1..9: note = Laminin 421/521
CSLANPATC

SEQ ID NO: 636 - protein, 12 aa, synthetic construct
  REGION 1..12: note = Laminin 421/521
GEPLTRFPNSDS

SEQ ID NO: 637 - protein, 12 aa, synthetic construct
  REGION 1..12: note = Laminin 421/521
KPILWNRSLNAL

SEQ ID NO: 638 - protein, 12 aa, synthetic construct
  REGION 1..12: note = Laminin 421/521
GFADVIHRKWSS

SEQ ID NO: 639 - protein, 12 aa, synthetic construct
  REGION 1..12: note = Laminin 421/521
HQSLAGVPWSRH

SEQ ID NO: 640 - protein, 12 aa, synthetic construct
  REGION 1..12: note = Laminin 421/521
VPVFLDQARVMK

SEQ ID NO: 641 - protein, 12 aa, synthetic construct
  REGION 1..12: note = Laminin 421/521
DDLLTAPRLGVW

SEQ ID NO: 642 - protein, 12 aa, synthetic construct
  REGION 1..12: note = Laminin 421/521
SDRGKTVLHGQI

SEQ ID NO: 643 - protein, 9 aa, synthetic construct
  REGION 1..9: note = Laminin 421/521
CGITQTTTC

SEQ ID NO: 644 - protein, 9 aa, synthetic construct
  REGION 1..9: note = Laminin 421/521
CHQRQQTYC

SEQ ID NO: 645 - protein, 12 aa, synthetic construct
  REGION 1..12: note = Laminin 421/521
EYWHQRGSWFHR

SEQ ID NO: 646 - protein, 7 aa, synthetic construct
  REGION 1..7: note = Laminin 421/521
YTPRNQL

SEQ ID NO: 647 - protein, 9 aa, synthetic construct
  REGION 1..9: note = Laminin 421/521
CTKSPSNSC

SEQ ID NO: 648 - protein, 7 aa, synthetic construct
  REGION 1..7: note = Laminin 421/521
WAFKAPQ

SEQ ID NO: 649 - protein, 12 aa, synthetic construct
  REGION 1..12: note = Laminin 421/521
GSLDMGNNKQPV

SEQ ID NO: 650 - protein, 12 aa, synthetic construct
  REGION 1..12: note = Laminin 421/521
AVVSQNQMSQQK

SEQ ID NO: 651 - protein, 9 aa, synthetic construct
  REGION 1..9: note = Laminin 421/521
CLNQKWEAC

SEQ ID NO: 652 - protein, 7 aa, synthetic construct
  REGION 1..7: note = Laminin 421/521
WPVRNLL

SEQ ID NO: 653 - protein, 12 aa, synthetic construct
  REGION 1..12: note = Laminin 421/521
GYRSFIHENWSI

SEQ ID NO: 654 - protein, 12 aa, synthetic construct
  REGION 1..12: note = Laminin 421/521
SGWEGKTHQGVR

SEQ ID NO: 655 - protein, 12 aa, synthetic construct
  REGION 1..12: note = Laminin 421/521
LQSNAADHHQGM

SEQ ID NO: 656 - protein, 12 aa, synthetic construct
  REGION 1..12: note = Laminin 421/521
ATSKYPNSWAQT

SEQ ID NO: 657 - protein, 12 aa, synthetic construct
  REGION 1..12: note = Laminin 421/521
DRLTPITWDWSR

SEQ ID NO: 658 - protein, 12 aa, synthetic construct
  REGION 1..12: note = Laminin 421/521
WTKHNTPAHHLS

SEQ ID NO: 659 - protein, 12 aa, synthetic construct
  REGION 1..12: note = Laminin 421/521
WNANYGTMTRHA

SEQ ID NO: 660 - protein, 7 aa, synthetic construct
  REGION 1..7: note = Laminin 421/521
HASNQRV

SEQ ID NO: 661 - protein, 7 aa, synthetic construct
  REGION 1..7: note = Laminin 421/521
GRTIHSM

SEQ ID NO: 662 - protein, 12 aa, synthetic construct
  REGION 1..12: note = Laminin 421/521
RLVTASMVSPSF

SEQ ID NO: 663 - protein, 12 aa, synthetic construct
  REGION 1..12: note = Laminin 421/521
DGQVSDHSYVQW

SEQ ID NO: 664 - protein, 12 aa, synthetic construct
  REGION 1..12: note = Laminin 421/521
KISMNRLHANFT

SEQ ID NO: 665 - protein, 12 aa, synthetic construct
  REGION 1..12: note = Laminin 421/521
WDLPTLRKTKQA

SEQ ID NO: 666 - protein, 7 aa, synthetic construct
  REGION 1..7: note = Laminin 421/521
WVAAERR

SEQ ID NO: 667 - protein, 12 aa, synthetic construct
  REGION 1..12: note = Laminin 421/521
MTHSAHNSQKTN

SEQ ID NO: 668 - protein, 7 aa, synthetic construct
  REGION 1..7: note = Laminin 421/521
WGLKLPH

SEQ ID NO: 669 - protein, 7 aa, synthetic construct
  REGION 1..7: note = Laminin 421/521
QGWFRHS

SEQ ID NO: 670 - protein, 7 aa, synthetic construct
  REGION 1..7: note = Laminin 421/521
IPVGERK

SEQ ID NO: 671 - protein, 9 aa, synthetic construct
  REGION 1..9: note = Laminin 421/521
CMNKIQRDC

SEQ ID NO: 672 - protein, 12 aa, synthetic construct
  REGION 1..12: note = Laminin 421/521
KALGYSLVGGEW

SEQ ID NO: 673 - protein, 12 aa, synthetic construct
  REGION 1..12: note = Laminin 421/521
APKVTNARPTQL

SEQ ID NO: 674 - protein, 12 aa, synthetic construct
  REGION 1..12: note = Laminin 421/521
AWVEAQNASNPS

SEQ ID NO: 675 - protein, 9 aa, synthetic construct
  REGION 1..9: note = Laminin 421/521
CSKHNHSRC

SEQ ID NO: 676 - protein, 12 aa, synthetic construct
  REGION 1..12: note = Laminin 421/521
GKPDHRLVSLWR

SEQ ID NO: 677 - protein, 9 aa, synthetic construct
  REGION 1..9: note = Laminin 421/521
CSHHKLKQC

SEQ ID NO: 678 - protein, 12 aa, synthetic construct
  REGION 1..12: note = Laminin 421/521
GGYDLPWANWQN

SEQ ID NO: 679 - protein, 12 aa, synthetic construct
  REGION 1..12: note = nerve-binding peptide
GWQMFPPMQNTR

SEQ ID NO: 680 - protein, 12 aa, synthetic construct
  REGION 1..12: note = nerve-binding peptide
SSLYMNGWSRDQ

SEQ ID NO: 681 - protein, 12 aa, synthetic construct
  REGION 1..12: note = nerve-binding peptide
YAGQNGWMRSDL

SEQ ID NO: 682 - protein, 12 aa, synthetic construct
  REGION 1..12: note = nerve-binding peptide
VERLNWPKRMQS

SEQ ID NO: 683 - protein, 12 aa, synthetic construct
  REGION 1..12: note = nerve-binding peptide
WDVAWLERRALH

SEQ ID NO: 684 - protein, 12 aa, synthetic construct
  REGION 1..12: note = nerve-binding peptide
VRLPMLASMGHQ

SEQ ID NO: 685 - protein, 12 aa, synthetic construct
  REGION 1..12: note = nerve-binding peptide
DRGLTILTDDLR

SEQ ID NO: 686 - protein, 12 aa, synthetic construct
  REGION 1..12: note = nerve-binding peptide
GWNEATNSARHT

SEQ ID NO: 687 - protein, 12 aa, synthetic construct
  REGION 1..12: note = nerve-binding peptide
EFARFRVFGVAE

SEQ ID NO: 688 - protein, 12 aa, synthetic construct
  REGION 1..12: note = nerve-binding peptide
NRIGHGTATGTL

SEQ ID NO: 689 - protein, 12 aa, synthetic construct
  REGION 1..12: note = nerve-binding peptide
VTDMLGIPKLHA

SEQ ID NO: 690 - protein, 12 aa, synthetic construct
  REGION 1..12: note = nerve-binding peptide
DELVGGAPWYKS

SEQ ID NO: 691 - protein, 12 aa, synthetic construct
  REGION 1..12: note = nerve-binding peptide
GLLREPDELMFL

SEQ ID NO: 692 - protein, 12 aa, synthetic construct
  REGION 1..12: note = nerve-binding peptide
QNIVENFIDNER

SEQ ID NO: 693 - protein, 12 aa, synthetic construct
  REGION 1..12: note = nerve-binding peptide
SGEYNLIKPNLL

SEQ ID NO: 694 - protein, 12 aa, synthetic construct
  REGION 1..12: note = nerve-binding peptide
EESNAKLELLSR

SEQ ID NO: 695 - protein, 12 aa, synthetic construct
  REGION 1..12: note = nerve-binding peptide
GWANSITYRNLY

SEQ ID NO: 696 - protein, 12 aa, synthetic construct
  REGION 1..12: note = nerve-binding peptide
NYAVGDRITKNI

SEQ ID NO: 697 - protein, 12 aa, synthetic construct
  REGION 1..12: note = nerve-binding peptide
HGIVFSKRPFSN

SEQ ID NO: 698 - protein, 12 aa, synthetic construct
  REGION 1..12: note = nerve-binding peptide
FDPLSRNVPHKT

SEQ ID NO: 699 - protein, 12 aa, synthetic construct
  REGION 1..12: note = nerve-binding peptide
NAVHRPDLNLLA

SEQ ID NO: 700 - protein, 12 aa, synthetic construct
  REGION 1..12: note = nerve-binding peptide
GMRTGDDSPKYP

SEQ ID NO: 701 - protein, 12 aa, synthetic construct
  REGION 1..12: note = nerve-binding peptide
IPGQTPRMPHSK

SEQ ID NO: 702 - protein, 12 aa, synthetic construct
  REGION 1..12: note = nerve-binding peptide
KTTSMTALTMGL

SEQ ID NO: 703 - protein, 12 aa, synthetic construct
  REGION 1..12: note = nerve-binding peptide
VNYQTPWNKHWY

SEQ ID NO: 704 - protein, 12 aa, synthetic construct
  REGION 1..12: note = nerve-binding peptide
VESGHAYSVSSW

SEQ ID NO: 705 - protein, 12 aa, synthetic construct
  REGION 1..12: note = nerve-binding peptide
VRSDMVSGGLQK

SEQ ID NO: 706 - protein, 12 aa, synthetic construct
  REGION 1..12: note = nerve-binding peptide
YANGIEAKDSSR

SEQ ID NO: 707 - protein, 12 aa, synthetic construct
  REGION 1..12: note = nerve-binding peptide
YAWDTVASRNYK

SEQ ID NO: 708 - protein, 12 aa, synthetic construct
  REGION 1..12: note = nerve-binding peptide
LQTHSITYRHER

SEQ ID NO: 709 - protein, 12 aa, synthetic construct
  REGION 1..12: note = nerve-binding peptide
SLGSSFVQGGEL

SEQ ID NO: 710 - protein, 12 aa, synthetic construct
  REGION 1..12: note = nerve-binding peptide
WEKHTGPLSRYL

SEQ ID NO: 711 - protein, 12 aa, synthetic construct
  REGION 1..12: note = nerve-binding peptide
AFRFEFQVPDSH

SEQ ID NO: 712 - protein, 12 aa, synthetic construct
  REGION 1..12: note = nerve-binding peptide
GHAHSLTQNPLF

SEQ ID NO: 713 - protein, 12 aa, synthetic construct
  REGION 1..12: note = nerve-binding peptide
GLTIMSSRHSLQ

SEQ ID NO: 714 - protein, 12 aa, synthetic construct
  REGION 1..12: note = nerve-binding peptide
NGMNWLQVHQLS

SEQ ID NO: 715 - protein, 12 aa, synthetic construct
  REGION 1..12: note = nerve-binding peptide
WSGGMEDNSNRL

SEQ ID NO: 716 - protein, 12 aa, synthetic construct
  REGION 1..12: note = nerve-binding peptide
AIMSYNSPWIQG

SEQ ID NO: 717 - protein, 12 aa, synthetic construct
  REGION 1..12: note = nerve-binding peptide
DVMQMFHQVHFY

SEQ ID NO: 718 - protein, 12 aa, synthetic construct
  REGION 1..12: note = nerve-binding peptide
QANLYYWDPSDL

SEQ ID NO: 719 - protein, 12 aa, synthetic construct
  REGION 1..12: note = nerve-binding peptide
QRATNDILIRGW

SEQ ID NO: 720 - protein, 12 aa, synthetic construct
  REGION 1..12: note = nerve-binding peptide
SPMANVWFNRLS

SEQ ID NO: 721 - protein, 12 aa, synthetic construct
  REGION 1..12: note = nerve-binding peptide
YSANSNPKKSLM

SEQ ID NO: 722 - protein, 12 aa, synthetic construct
  REGION 1..12: note = nerve-binding peptide
ALNHNGPVKTGL

SEQ ID NO: 723 - protein, 12 aa, synthetic construct
  REGION 1..12: note = nerve-binding peptide
DASQYPLVSGLW

SEQ ID NO: 724 - protein, 12 aa, synthetic construct
  REGION 1..12: note = nerve-binding peptide
EPWQSRDKVIEE

SEQ ID NO: 725 - protein, 12 aa, synthetic construct
  REGION 1..12: note = nerve-binding peptide
GLWDGKGRIVEV

SEQ ID NO: 726 - protein, 12 aa, synthetic construct
  REGION 1..12: note = nerve-binding peptide
GTVSYMVKMDNN

SEQ ID NO: 727 - protein, 12 aa, synthetic construct
  REGION 1..12: note = nerve-binding peptide
SLQLWNSEKVTT

SEQ ID NO: 728 - protein, 12 aa, synthetic construct
  REGION 1..12: note = nerve-binding peptide
WVGPDIIAQMRI

SEQ ID NO: 729 - protein, 12 aa, synthetic construct
  REGION 1..12: note = nerve-binding peptide
EIIVMSGSNVTN

SEQ ID NO: 730 - protein, 12 aa, synthetic construct
  REGION 1..12: note = nerve-binding peptide
HSGAYTIQASYK

SEQ ID NO: 731 - protein, 12 aa, synthetic construct
  REGION 1..12: note = nerve-binding peptide
MLKMERPGRMAY

SEQ ID NO: 732 - protein, 12 aa, synthetic construct
  REGION 1..12: note = nerve-binding peptide
SDHTFKRVMFRW

SEQ ID NO: 733 - protein, 12 aa, synthetic construct
  REGION 1..12: note = nerve-binding peptide
EMVPMKFLMMAK

SEQ ID NO: 734 - protein, 12 aa, synthetic construct
  REGION 1..12: note = nerve-binding peptide
NTEYSRYTSIWK

SEQ ID NO: 735 - protein, 12 aa, synthetic construct
  REGION 1..12: note = nerve-binding peptide
TDNGSFLGKLTS

SEQ ID NO: 736 - protein, 12 aa, synthetic construct
  REGION 1..12: note = nerve-binding peptide
TEWMVRASSIGE

SEQ ID NO: 737 - protein, 12 aa, synthetic construct
  REGION 1..12: note = nerve-binding peptide
VPVPKKMSLDPT

SEQ ID NO: 738 - protein, 12 aa, synthetic construct
  REGION 1..12: note = nerve-binding peptide
WPLKRFEQLPLK

SEQ ID NO: 739 - protein, 12 aa, synthetic construct
  REGION 1..12: note = nerve-binding peptide
MLRHVDLTEPNA

SEQ ID NO: 740 - protein, 12 aa, synthetic construct
  REGION 1..12: note = nerve-binding peptide
QSTLGVHMLRVS

SEQ ID NO: 741 - protein, 12 aa, synthetic construct
  REGION 1..12: note = nerve-binding peptide
RTVITNEMLLLV

SEQ ID NO: 742 - protein, 12 aa, synthetic construct
  REGION 1..12: note = nerve-binding peptide
SEPIEKGSRTIV

SEQ ID NO: 743 - protein, 12 aa, synthetic construct
  REGION 1..12: note = nerve-binding peptide
TLNARDQLSING

SEQ ID NO: 744 - protein, 12 aa, synthetic construct
  REGION 1..12: note = nerve-binding peptide
GTALNLQKDINK

SEQ ID NO: 745 - protein, 12 aa, synthetic construct
  REGION 1..12: note = nerve-binding peptide
SGWHLVMSMSIG

SEQ ID NO: 746 - protein, 12 aa, synthetic construct
  REGION 1..12: note = nerve-binding peptide
TSAHHDRTLAGS

SEQ ID NO: 747 - protein, 17 aa, synthetic construct
  REGION 1..17: note = HNP401-N-2 with GG linker
  SITE 1: note = 5-carboxyfluorescein
  MOD_RES 17: note = amidated
QVPWEEPYYVVKKSSGG

SEQ ID NO: 748 - protein, 20 aa, synthetic construct
  REGION 1..20: note = HNP401with GCC linker
  MOD_RES 1: note = acetyl
  MOD_RES 20: note = amidated
SGQVPWEEPYYVVKKSSGGC

SEQ ID NO: 749 - protein, 17 aa, synthetic construct
  REGION 1..17: note = NP41
  MOD_RES 1: note = acetyl
SHSNTQTLAKAPEHTGK

(19) United States
(12) Patent Application Publication
(10) Pub. No.: US 2025/0009909 A1
(43) Pub. Date: Jan. 9, 2025

(54) METHODS AND COMPOSITIONS FOR VISUALIZING A URETER IN A SURGICAL PROCEDURE

(71) Applicant: ALUME BIOSCIENCES, INC., San Diego, CA (US)

(72) Inventors: Quyen NGUYEN, San Diego, CA (US); Michael WHITNEY, San Diego, CA (US)

(21) Appl. No.: 18/780,117

(22) Filed: Jul. 22, 2024

Related U.S. Application Data
(63) Continuation of application No. 18/349,833, filed on Jul. 10, 2023.
Application No. 18/349,833 is a continuation of application No. PCT/US2022/012379, filed on Jan. 13, 2022.
(60) Provisional application No. 63/137,621, filed on Jan. 14, 2021.

(51) Int. Cl. A61K 49/00; C07K 7/06; C07K 7/08

(52) U.S. Cl. CPC A61K 49/0056; A61K 49/0021; A61K 49/0043; C07K 7/06; C07K 7/08

(57) ABSTRACT

Methods for visualizing a ureter, including fluorescent visualization of a ureter, as well as other target tissues, during a surgical procedure, comprising administering to a subject a fluorescent conjugate comprising a peptide and a fluorescent moiety.

### CROSS-REFERENCE TO RELATED APPLICATIONS

This application is a continuation of application of U.S. patent application Ser. No. 18/349,833, filed Jul. 10, 2023, which is a continuation of International Patent Application No. PCT/US2022/012379, filed Jan. 13, 2022, which claims the benefit of U.S. Provisional Application No. 63/137,621, filed Jan. 4, 2021, which applications are incorporated herein by reference in their entirety.

### STATEMENT REGARDING SEQUENCE LISTING

The contents of the electronic sequence listing (405C2_SeqListing.xml; Size: 915 kilobytes; and Date of Creation: Jun. 29, 2023) is herein incorporated by reference in its entirety.

### FIELD

The present invention relates generally to intraoperative imaging methods, and more particularly, to fluorescent visualization of ureters, as well as nerves, during surgical procedures.

### BACKGROUND

Ureters comprise a pair of multilayer muscular tubes—each about 10 to 12 inches long in an average adult-that transport urine from the kidneys to the bladder. Transport relies on peristaltic motion, arising from contractions in the muscular layer of the ureteral wall initiated by pacemakers. As the walls rhythmically tighten and relax, urine flows in a pulsatile motion through the ureters to the bladder.

Because they extend from the abdomen to the pelvis, ureters are in close proximity to many structures in the abdominopelvic cavity, such as gonadal and uterine vessels, iliac arteries, inferior mesenteric and sigmoid vessels, as well as the cervix, colon, and rectum. In addition, ureters are thin-walled tubes that are often in a collapsed state and are difficult to distinguish from surrounding tissue. Given their location and physical attributes, ureters are at significant risk of injury in many surgeries, including gynecological, urological, colorectal, and cardiovascular surgical procedures. See, e.g., Engelsgjerd and LaGrange, 2020, StatPearls, Treasure Island (FL): StatPearls Publishing; 2020 Jul. 10; Ferrara and Kann, 2019, Clin. Colon Rectal Surg. 32, 196-203; Ahn et al., 2019, Quant. Imaging Med. Surg. 9,1056-1065; Gild et al., 2018, Asian J. Urol. 5, 101-106. Indeed, ureteral injuries are often not noticed intraoperatively, resulting in delayed diagnoses and late treatments that in some cases can lead to long term complications such as kidney failure. Tan-Kim et al. 2015, J. Minimally Invasive Gynecol. 22, 1278-1286; Al-Awadi et al. 2005, Int. Urol. Nephrol. 37, 235-241.

Consequently, ureteral injuries significantly impact patient morbidity and concomitant healthcare costs. In the United States, about 600 thousand hysterectomies and 300 thousand colon surgeries are performed annually, with a ureteral injury rate as high as 2.5% and 7.6%, respectively. See Gild et al. 2018, Asian J. Urol. 5, 101-106; Doll et al. 2016, JAMA Surg. 151, 876-877; Tecluckdharry et al. 2015, Obstet. Gynecol. 126, 1161-1169; Briggs and Goldberg, 2011, Clin. Colon. Rectal Surg. 30, 130-135. In lower abdominal surgical procedures, iatrogenic ureteral injuries (IUIs) have estimated incidence rates ranging from 0.5-1% for cancer surgery, 0.3% to 2.5% for laparoscopic gynecological surgery, and up to 10% for gynecologic oncologic surgery. See, e.g., de Valk et al. 2019, Nat. Commun. 16, 3118; Anderson et al. 2015, Surg. Endosc. 29, 1406-1412; Engel et al. 2015, Curr. Opin. Urol. 25, 331-335; Minas et al. 2014, Obstet. Gynecol. 16, 19-28; Zafar et al. 2014, JSLS. 18, c2014.00158; Silva et al. 2012, Asian J. Endosc. Surg. 5, 1050110; Delacroix and Winters, 2010, Clin. Colon Rectal Surg. 23,104-112. The collective economic impact of ureteral injuries in just the United States alone exceeds $1 billion, reflecting an average hospital stay of 4 days and costs exceeding $30,000 per injury.

Several approaches have been explored to help reduce the risk of ureteral injuries during surgery. These include urological procedures, such as inserting a stent or an illuminated catheter into a ureter, but these can disrupt the workflow of surgery and introduce unwanted delays, for example by requiring intraoperative consultation with urologists or other specialists. See e.g., Wood et al. 1996, J. Am. Assoc. Gynecol. Laparosc, 3, 393-397; Chahin et al. 2002, JSLS 6, 49-52. Other approaches include administering or injecting radioactive dyes or fluorophores (or certain conjugates) to visualize the ureters. Such injection procedures, however, can present additional risks and complexities, and some agents can have limited sensitivity, intensity, and duration of action. See, e.g., Ikeda et al., 2017, Am. J. Physiol. Renal Physiol. 312, F629-F639; Al-Taher et al., 2016, J. Laparoendosc. Adv. Surg. Tech. A. 26, 870-875; Verbeek et al., 2014, Ann. Surg. Oncol. 21, S528-S537; Hyun et al. 2012, Contrast Media Mol. Imaging 7, 516-524; Choi et al., 2011, Angew Chem. Int. Ed. Engl. 50, 6258-6263; Tanaka, et al. 2007, J. Urol. 178, 2197-2201; Cadeddu et al., 2001, J. Endourology 15, 111-116.

There remains a need in the art to identify compositions with desirable properties that include the visualization of a ureter during surgical procedures. The present invention meets these and other needs in the art by providing peptide compositions, particularly fluorescent peptide conjugates, which allow sensitive and prolonged ureter visualization following administration, and can further support nerve visualization, including concurrent nerve and ureter visualization.

### SUMMARY

Disclosed herein are methods for visualizing a ureter, including methods for fluorescent visualization of a ureter during a surgical procedure.

In one aspect, the disclosure is directed to a method for visualizing a ureter in a subject, the method comprising: (a) administering to the subject an effective amount of a fluorescent conjugate, wherein the fluorescent conjugate comprises a peptide and a fluorescent moiety, and (b) detecting fluorescence of the fluorescent moiety in the ureter following administration. In embodiments, the subject is undergoing a surgical procedure.

In another aspect, the disclosure is directed to a method comprising: (a) administering to a subject undergoing a surgical procedure an effective amount of a fluorescent conjugate, wherein the fluorescent conjugate comprises a peptide and a fluorescent moiety; and (b) visualizing a ureter in the subject by detecting fluorescence of the fluorescent moiety in the ureter.

In another aspect, the disclosure is directed to a method of reducing injury to a ureter, the method comprising: (a) identifying a subject at a risk of a ureteral injury prior to a surgical procedure; (b) administering to the subject undergoing the surgical procedure an effective amount of a fluorescent conjugate, wherein the fluorescent conjugate comprises a peptide and a fluorescent moiety; and (c) visualizing the ureter by detecting fluorescence of the fluorescent moiety in the ureter. In embodiments, the ureteral injury arises from ligation, angulation, transection, laceration, crush, ischemia, or resection.

In embodiments of the methods, the peptide can be a nerve targeting peptide. In any of the methods, the peptide can consist of or comprise any of the amino acid sequences disclosed herein, including those set forth in International Patent Application No. PCT/US2018/045054 and International Patent Application No. PCT/US21/61821. In embodiments, the peptide of the fluorescent conjugate comprises an amino acid sequence selected from the group consisting of SEQ ID NOS: 1-14, 16, 18-82, and 84-748.

In embodiments of any of the methods, the fluorescent moiety can be conjugated directly or indirectly via a linker to the N-terminus, C-terminus, or both N-terminus and C-terminus of the peptide. In embodiments of any of the methods, the fluorescent moiety is selected from the group consisting of a fluorescent protein, a fluorescent peptide, a fluorescent dye, or combinations thereof, as disclosed herein.

In embodiments, any of the methods can further comprise detecting fluorescence of the fluorescent moiety within a period of at least 1 hour, at least 2 hours, at least 3 hours, at least 4 hours, at least 5 hours, at least 6 hours, at least 7 hours, or at least 8 hours, following administration. In embodiments, the methods can further comprise detecting fluorescence of the fluorescent moiety for a period of more than 1 hour, more than 2 hours, more than 3 hours, more than 4 hours, more than 5 hours, more than 6 hours, more than 7 hours, or more than 8 hours, following administration.

In embodiments, the fluorescent conjugate in any of the methods is not further administered to the subject following initial administration. In any of the methods, detecting fluorescence of the fluorescent moiety in the ureter can comprise detecting urine flow in the ureter, and more particularly detecting peristaltic flow of urine in the ureter. In any of any of the methods, the fluorescent conjugate may be administered intravenously. In any of any of the methods, the fluorescent conjugate may be administered orally. In any of the methods, the fluorescent conjugate can be administered to a subject prior to a surgical procedure.

In embodiments, the surgical procedure in any of the methods can comprise a gynecological, urological, colorectal, or cardiovascular surgical procedure; can involve the abdomen or pelvis; can be performed on a kidney, bladder, prostate, uterus, male or female reproductive system, rectum, colon, small intestine, or large intestine; can be an open surgical procedure, a laparoscopic surgical procedure, a microscopic procedure, or an endoscopic procedure; or can be a cancer surgical procedure, and more particularly, a prostate cancer surgical procedure or a colorectal cancer procedure.

In embodiments, the fluorescent conjugate in any of the methods comprises a fluorescent moiety selected from the group consisting of: a fluorescent protein, a fluorescent peptide, a fluorescent dye, or combinations thereof, as disclosed herein.

In embodiments, any of the methods comprise administering the fluorescent conjugate in a pharmaceutical composition, which can comprise a pharmaceutically acceptable excipient.

In embodiments, the fluorescent conjugates can allow nerve visualization, as well as concomitant (or concurrent) nerve and ureter visualization in a subject. In embodiments, the fluorescent conjugates can confer nerve visualization and ureter visualization in a subject at different times during a surgical procedure.

### BRIEF DESCRIPTION OF THE DRAWING

FIG. 1: Bright field and fluorescence overlay image of a ureter in a living wild type mouse following intravenous administration of about 2000 nanomoles of a fluorescent conjugate (SEQ ID NO:747). Images were obtained 2 hours following administration using a customized fluorescence detection microscope. Left panel: White reflectance image. Right panel: fluorescence overlay.

### DETAILED DESCRIPTION

Disclosed herein are methods for visualization of a ureter, and more particularly, methods for fluorescent visualization of a ureter, by administering a fluorescent conjugate (also referred to herein as a “conjugate”) comprising a peptide and a fluorescent moiety. It has been discovered that metabolism and elimination of certain fluorescent conjugates, including ones comprising a nerve targeting peptide, support sensitive and prolonged detection of fluorescence during passage in the ureter from the kidneys to the bladder. Advantageously, use of the fluorescent conjugates herein allows ureter detection both early and later after administration. These properties underscore the broad utility of such conjugates, conferring ureter visualization for shorter periods, as well as longer periods, following administration.

Accordingly, in embodiments, the methods comprise visualizing a ureter in a subject by detecting the fluorescent moiety in the ureteral flow of urine, and more particularly in the peristaltic ureteral flow of urine. The fluorescent conjugate can be administered intravenously, and can be administered to a subject prior to a surgical procedure.

In embodiments, the peptide in the fluorescent conjugate comprises a nerve targeting peptide, and more particularly, comprises a nerve targeting peptide disclosed in International Patent Application No. PCT/US2018/045054 or in International Patent Application No. PCT/US21/61821, both of which are incorporated herein in their entirety. Accordingly, in embodiments, fluorescent conjugates disclosed herein can allow concurrent detection of ureters and nerves.

In embodiments, the peptide in the fluorescent conjugate comprises the amino acid sequence QVPWEEPYYVVKKSSGG (HNP401-N-2 with GG linker; SEQ ID NO:21), as disclosed in International Patent Publication No. WO2019028281A2. In embodiments, the fluorescent conjugate is 5FAM-QVPWEEPYYVVKKSSGG-NH2 (HNP401-N-2 with GG linker; SEQ ID NO:747).

The invention may be more fully appreciated by reference to the following description, including the examples. Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described herein. In addition, the materials, methods, and examples are illustrative only and not intended to be limiting.

For the sake of brevity, all documents, or portions of documents, cited in this application including, without limitation, patents, patent applications, articles, books, manuals, and treatises are hereby expressly incorporated by reference in their entirety for any purpose. Citation of any such publication, however, shall not be construed as an admission that it is prior art to the present invention

The use of heading and subheadings in sections of this specification is solely for organizational purposes and convenience of reference and is not to be construed as limiting the subject matter described, which is to be construed by reference to the specification as a whole. For example, those of skill in the art will appreciate the usefulness of combining various aspects from different headings and sections as appropriate according to the spirit and scope of the invention described herein.

As will become apparent to one of ordinary skill in the art after reading this document, the illustrated embodiments and their various alternatives may be implemented without confinement to the illustrated examples

### I. Definitions

As used herein, he terms “a” and “an” refer to “one or more” of the enumerated components. The use of the alternative (e.g., “or”) should be understood to mean either one, both, or any combination thereof of the alternatives. In addition, it should be understood that the individual compounds, or groups of compounds, derived from the various combinations of the structures and substituents described herein, are disclosed by the present application to the same extent as if each compound or group of compounds were set forth individually. Thus, selection of particular structures or particular substituents is within the scope of the present disclosure.

As used herein, the term “about” or “approximately” means a range of values including the specified value, which a person of ordinary skill in the art would consider reasonably similar to the specified value. In embodiments, “about” means within a standard deviation using measurements generally acceptable in the art. In embodiments, “about” means a range extending to +/−10% of the specified value. In embodiments, “about” means the specified value.

It is understood that, whether the term “about” is used explicitly or not, every quantity given herein is meant to refer to both the actual given value and the approximation of such given value that would reasonably be inferred based on the ordinary skill in the art, including equivalents and approximations due to the experimental and/or measurement conditions for such given value. Accordingly, for any embodiment of the disclosure in which a numerical value is prefaced by “about” or “approximately,” the disclosure includes an embodiment in which the exact value is recited. Conversely, for any embodiment of the disclosure in which a numerical value is not prefaced by “about” or “approximately”, the disclosure includes an embodiment in which the value is prefaced by “about” or “approximately”.

The terms “comprising” and “including” are used herein in their open, non-limiting sense. Other terms and phrases used in this document, and variations thereof, unless otherwise expressly stated, should be construed as open ended, as opposed to limiting. As examples of the foregoing: the term “example” is used to provide exemplary instances of the item in discussion, not an exhaustive or limiting list thereof. Adjectives such as “conventional,” “normal,” “known” and terms of similar meaning should not be construed as limiting the item described to a given time period or to an item available as of a given time, but instead should be read to encompass conventional, or normal technologies that may be available or known now or at any time in the future. Likewise, where this document refers to technologies that would be apparent or known to one of ordinary skill in the art, such technologies encompass those apparent or known to the skilled artisan now or at any time in the future.

As used herein, the following terms have the meanings ascribed to them unless specified otherwise.

The terms “polypeptide,” “peptide,” and “protein” are used interchangeably herein to refer to a polymer of amino acid residues. The terms apply to naturally occurring amino acid polymers as well as amino acid polymers in which one or more amino acid residues is a non-naturally occurring amino acid (e.g., an amino acid analog). The terms encompass amino acid chains of any length, including full length proteins (i.e., nerve targeting molecules), wherein the amino acid residues are linked by covalent peptide bonds. As used herein, the term “peptide” refers to a polymer of amino acid residues typically ranging in length from 2 to about 50 residues. In certain embodiments the peptide ranges in length from about 2, 3, 4, 5, 7, 9, 10, or 11 residues to about 50, 45, 40, 45, 30, 25, 20, or 15 residues. In certain embodiments the peptide ranges in length from about 8, 9, 10, 11, or 12 residues to about 15, 20 or 25 residues. Where an amino acid sequence is provided herein, L-, D-, or beta amino acid versions of the sequence are also contemplated as well as retro, inversion, and retro-inversion isoforms. Peptides also include amino acid polymers in which one or more amino acid residues is an artificial chemical analogue of a corresponding naturally occurring amino acid, as well as to naturally occurring amino acid polymers. In addition, the term applies to amino acids joined by a peptide linkage or by other modified linkages (e.g., where the peptide bond is replaced by an α-ester, a/3-ester, a thioamide, phosphonamide, carbamate, hydroxylate, and the like (sec, e.g., Spatola 1983, Chem. Biochem. Amino Acids and Proteins 7:267-357), where the amide is replaced with a saturated amine (sec, e.g., Skiles et al., U.S. Pat. No. 4,496,542, which is incorporated herein by reference, and Kaltenbronn et al., 1990, pp. 969-970 in Proc. 11th American Peptide Symposium, ESCOM Science Publishers, The Netherlands, and the like)).

Accordingly, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. An amino acid may be an L- or D-amino acid. Amino acid analogs refer to compounds that have the same basic chemical structure as a naturally occurring amino acid, i.e., a carbon that is bound to a hydrogen, a carboxyl group, an amino group, and an R group, e.g., homoserine, norleucine, methionine sulfoxide. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUP AC-IUB Biochemical Nomenclature Commission. Nucleotides, likewise, may be referred to by their commonly accepted single-letter codes.

One of skill will recognize that individual substitutions, deletions or additions to a peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (sec, e.g., Creighton, Proteins (1984)).

“Sequence identity” (or “identity”) as used herein, refers to the percentage of amino acid residues in a single given sequence that are identical with the amino acid residues in another reference polypeptide sequence after aligning the sequences and introducing gaps, if necessary, to achieve the maximum percent sequence identity, and not considering any conservative substitutions as part of the sequence identity. “Sequence homology” (or “homology”), as used herein, refers to the percentage of amino acid residues in a single given sequence that are identical or have similar chemical properties to the amino acid residues in another reference polypeptide sequence after aligning the sequences and introducing gaps, if necessary, to achieve the maximum percent sequence homology. Thus, in contrast to sequence identity, sequence homology considers any conservative substitutions (as described herein) as part of the sequence alignment. Sequence identity or sequence homology can be determined using well known methods, including publicly available computer programs such as the NCBI BLAST 2.0 software as defined by Altschul et al. 1997, Nucl. Acids Res. 25, 3389-3402. These programs optimally align sequences using default gap weights in order to produce the highest level of sequence identity or sequence homology between the given and reference sequences.

As used herein, the terms “label” refers to a molecule that facilitates the visualization and/or detection of a targeting molecule disclosed herein. In embodiments, the label is a fluorescent moiety.

The terms “individual,” “patient,” or “subject” are used interchangeably. As used herein, they mean any mammal (i.e. species of any orders, families, and genus within the taxonomic classification animalia: chordata: vertebrata: mammalia). In embodiments, the mammal is a cow, horse, sheep, pig, cat, dog, goat, mouse, rat, rabbit, guinea pig, non-human primate, or human. In embodiments, the mammal is a human subject, and more particularly, a human subject undergoing a surgical procedure. None of the terms require or are limited to situations characterized by the supervision (e.g. constant or intermittent) of a health care worker (e.g. a doctor, a registered nurse, a nurse practitioner, a physician's assistant, an orderly, or a hospice worker).

The terms “administer,” “administering”, “administration,” and the like, as used herein, refer to the methods that may be used to enable delivery of agents or compositions to the desired site of biological action. These methods include, but are not limited to parenteral injection (e.g., intravenous, subcutaneous, intraperitoneal, intramuscular, intravascular, intrathecal, intravitreal, infusion, or local). Administration techniques that are optionally employed with the agents and methods described herein, include e.g., those discussed in in Gennaro: Remington: The Science and Practice of Pharmacy, 21^{st} Ed. (Lippincott Williams & Wilkins, 2005) and in Goodman & Gilman's The Pharmacological Basis of Therapeutics (McGraw-Hill Professional, 2005). In embodiments, administration is via systemic intravenous injection into human patients.

The term “effective amount” is interchangeable with a “therapeutically effective amount” and means an amount sufficient to accomplish a stated purpose, e.g., to achieve the effect for which it is administered, such as allowing fluorescent visualization of a ureter intraoperatively (during the course of surgery, a surgical procedure, a surgical method, or an operation). An example of an “effective amount” is an amount sufficient to contribute to the treatment, prevention, or reduction of an adverse event caused by a surgical or medical procedure, such as an iatrogenic ureteral injury (IUI) during an abdominal surgical procedure. The desired effect may, but does not necessarily, occur by administration of one dose. It may also occur after administration of a series of doses. Thus, an effective amount may be administered in one or more administrations. Determination of a therapeutically effective amount of a composition is within the capabilities of a skilled artisan, particularly in light of the detailed disclosure herein. For example, a dose for humans can be formulated to achieve a concentration that has been found to be effective in animals. The dosage in humans can be adjusted by monitoring the effectiveness of an agent or composition disclosed herein and adjusting the dosage upwards or downwards. Adjusting the dose to achieve maximal efficacy in humans based on the methods described herein and other methods is well within the capabilities of the ordinarily skilled artisan.

The term “pharmaceutically acceptable” as used herein, refers to a material that does not abrogate the biological activity or properties of the agents and compositions described herein, and is relatively nontoxic (i.e., the toxicity of the material significantly outweighs the benefit of the material). In some instances, a pharmaceutically acceptable material may be administered to an individual without causing significant undesirable biological effects or significantly interacting in a deleterious manner with any of the components of the composition in which it is contained.

The term “surgery” as used herein, refers to any methods or procedures that may be used to manipulate, change, or cause an effect by a physical intervention. Surgical methods and procedures include, but are not limited to, open surgery, microscopic surgery, endoscopic surgery, laparoscopic surgery, minimally invasive surgery, robotic surgery. In some instances, the subject of the surgery is a human subject or human patient. A surgical procedure may include a gynecological, urological, colorectal, or cardiovascular surgical procedure. A surgical procedure may also include, but is not limited to, an abdominal or pelvic procedure. In certain instances, the surgical procedure is performed on a kidney, bladder, prostate, uterus, male or female reproductive system, rectum, colon, small intestine, or large intestine. A surgical procedure may be a cancer surgical procedure, including, for example, a prostate cancer surgical procedure or a colorectal procedure. A surgical procedure may also include a procedure that presents a risk of ureter injury, and that in some cases, may affect nerves as well, as the compositions and methods described herein can support intraoperative nerve visualization, including concurrent nerve and ureter visualization in a subject during surgery.

### II. Methods

The present disclosure provides methods of visualizing a ureter, particularly during surgery, with the use of a fluorescent conjugate that comprises a peptide linked to a fluorescent moiety, i.e., a labeled peptide. In embodiments, the peptide comprises a nerve targeting peptide. In embodiments, the fluorescent conjugates (and compositions thereof) described herein are useful as surgical adjuncts in such methods to reduce the likelihood of ureter injuries, and in particular iatrogenic ureteral injuries (IUI), during surgical procedures.

As described further herein, it has been observed that nerve-targeting fluorescent conjugates can be cleared by renal excretion under conditions that allow significant and extended fluorescence during passage through the ureter. Consequently, ureter visualization can be achieved by detecting the fluorescent moiety in the flow of urine through a ureter, and more particularly, the peristaltic flow of urine through a ureter. Ureter visualization can persist for an extended period, lasting several hours or more after administration of certain fluorescent conjugates.

Accordingly, in embodiments, the disclosure provides methods of visualizing a ureter in a subject using any of the molecules and compositions described herein. In embodiments, the subject is undergoing a procedure, such as a diagnostic procedure, a medical procedure, or a surgical procedure.

In one aspect, the methods comprise: (a) administering to a subject an effective amount of a fluorescent conjugate, wherein the fluorescent conjugate comprises a peptide and a fluorescent moiety; and (b) detecting fluorescence of the fluorescent moiety in the ureter of the subject following administration.

In another aspect, the methods comprise: (a) administering to a subject undergoing a surgical procedure an effective amount of a fluorescent conjugate, wherein the fluorescent conjugate comprises a peptide and a fluorescent moiety; and (b) visualizing a ureter in the subject by detecting fluorescence of the fluorescent moiety in the ureter.

In another aspect, the methods comprise: (a) identifying a subject at a risk of a ureteral injury prior to a surgical procedure; (b) administering to the subject undergoing the surgical procedure an effective amount of a fluorescent conjugate, wherein the fluorescent conjugate comprises a peptide and a fluorescent moiety; and (c) visualizing the ureter by detecting fluorescence of the fluorescent moiety in the ureter. As used herein, a subject at risk of ureteral injury includes a subject that is in need of a diagnostic, medical, or surgical procedure described herein.

In embodiments, the fluorescent conjugate is initially administered prior to the surgical procedure. In embodiments, the fluorescent conjugate is not administered following the initial administration. In embodiments, the fluorescent conjugate is administered during the surgical procedure. In embodiments, the fluorescent conjugate is administered prior to and during the surgical procedure.

In embodiments, a surgical procedure comprises a gynecological, urological, colorectal, or cardiovascular surgical procedure. In embodiments, the surgical procedure comprises an abdominal or pelvic procedure. In embodiments, the surgical procedure is performed on a kidney, bladder, prostate, uterus, male or female reproductive system, rectum, colon, small intestine, or large intestine. In embodiments, the surgical procedure is an open surgical procedure, a laparoscopic surgical procedure, a microscopic procedure, or an endoscopic procedure. In embodiments, the surgical procedure is a cancer surgical procedure, and more particularly, a prostate cancer surgical procedure.

In embodiments of any of these methods, the fluorescent conjugate is administered to a subject, as described herein, including humans and mammals (e.g., mice, rats, pigs, cats, dogs, and horses). In embodiments, the subject is are mammal. In embodiments, the subject is a primate. In embodiments, the subject is a human. In embodiments, the human subject is a pediatric subject (21 years and younger), an adult subject (22 years to 65 years), or a geriatric subject (65 years and above).

The fluorescent conjugate can be administered by any route, as described further herein. In embodiments, the fluorescent conjugate is administered intravenously. In embodiments, the fluorescent conjugate is administered orally. In embodiments, a fluorescent conjugate administered by any route may be administered as a pharmaceutical composition, as described herein, which may include a pharmaceutically acceptable excipient.

A fluorescent moiety of a fluorescent conjugate can be detected in a ureter by any suitable method known in the art. For example, a fluorescent moiety may be detected by exciting the fluorophore in the surgical field with the appropriate wavelength of light and detecting the resulting fluorescence (and surgical field) using a combination of filters, lenses and cameras.

In embodiments of the methods, the fluorescent moiety is detected in a ureter by a medical device adapted for fluorescence guided surgery (FGS). Such adapted devices may include, but are not limited to, hand held or mounted devices used in open surgery; minimally invasive devices, such as laparoscopes and endoscopes used in abdominal and pelvic procedures; robotic surgical systems used in prostate surgeries; and other devices, such as surgical loupes. Devices may provide real time synchronous information from bright field illumination and fluorescence emission. One or more light sources can be used to excite and illuminate the sample or surgical field. Light can be collected to produce final images, using optical filters customized to the emission spectrum of the fluorescent moiety, imaging lenses, and digital cameras (such as CCD and CMOS detector systems). Live video processing can also be performed to enhance contrast during fluorescence detection and improve the sensitivity of the fluorescent signal.

In embodiments of the methods, fluorescence of the fluorescent moiety is detected in a ureter within a period of at least 1 hour, at least 2 hours, at least 3 hours, at least 4 hours, at least 5 hours, at least 6 hours, at least 7 hours, or at least 8 hours, following administration.

In embodiments of the methods, fluorescence of the fluorescent moiety is detected in a ureter for a period of more than 1 hour, more than 2 hours, more than 3 hours, more than 4 hours, more than 5 hours, more than 6 hours, more than 7 hours, or more than 8 hours, following administration.

In embodiments of the methods, fluorescence of the fluorescent moiety is detected in a ureter after 30 minutes, after 1 hour, after 2 hours, after 3 hours, after 4 hours, after 5 hours, after 6 hours, after 7 hours, or after more than 8 hours, following administration.

In embodiments of the methods, the fluorescent conjugates can allow concomitant nerve detection, as well as ureter visualization, including intraoperative nerve and ureter visualization. Such a nerve can include a human nerve, as disclosed, for example, in the '054 Application, such as motor nerves, sensory nerves, sympathetic and parasympathetic nerves, peripheral nerves, and autonomic nerves.

More generally, the methods, conjugates, and compositions described herein can also encompass open surgical procedures, laparoscopic surgical procedures, microscopic procedures, and endoscopic procedure.

In addition, the methods, conjugates, and compositions described herein can also be used in surgical procedures, in which visualization of nerves, as well as ureters, is desirable.

### III. Fluorescent Conjugates

Fluorescent conjugates for use in the present methods comprise peptides, and more particularly, nerve-targeting peptides, linked to fluorescent moieties (or a peptide otherwise comprising a fluorescent label).

### Peptides

In embodiments, the nerve targeting peptide is any peptide, including any deletion or linker variant, as disclosed in International Patent Application No. PCT/US2018/045054 (“the '054 Application,” published as WO2019028281A2), which is incorporated herein in its entirety). Accordingly, such peptides comprise, or consist of, any one of the following amino acid sequences (peptide sequences) listed in Table 1 (SEQ ID NOS: 1-17, 20-28, and 96-102):

TABLE 1

| | SEQ | |
|---|---|---|
| | ID | |
| Name | NO: | Amino Acid Sequence |
| HNP401 | 1 | SGQVPWEEPYYVVKKSS |
| | | (SEQ ID NO: 1) |
| HNP402 | 2 | WEYHYVDLNWTSQHPQ |
| | | (SEQ ID NO: 2) |
| HNP403 | 3 | DLPDIIWDENWETA |
| | | (SEQ ID NO: 3) |
| HNP401 with | 4 | Ac-SGQVPWEEPYYVVKKSSGGC |
| GGC linker | | (SEQ ID NO: 4) |
| HNP402 with | 5 | Ac-WEYHYVDLNWTSQHPQGGC |
| GGC linker | | (SEQ ID NO: 5) |
| HNP403 with | 6 | Ac-DLPDIIWDFNWETAGGC |
| GGC linker | | (SEQ ID NO: 6) |
| HNP401-N-2 | 7 | Ac-QVPWEEPYYVVKKSSGGC |
| with GGC | | (SEQ ID NO: 7) |
| linker | | |
| HNP401-N-4 | 8 | Ac-PWEEPYYVVKKSSGGC |
| with GGC | | (SEQ ID NO: 8) |
| linker | | |
| HNP401-N-6 | 9 | Ac-EEPYYVVKKSSGGC |
| with GGC | | (SEQ ID NO: 9) |
| linker | | |
| HNP401-N-8 | 10 | Ac-PYYVVKKSSGGC |
| with GGC | | (SEQ ID NO: 10) |
| linker | | |
| HNP401-C-2 | 11 | Ac-SGQVPWEEPYYVVKKGGC |
| with GGC | | (SEQ ID NO: 11) |
| linker | | |
| HNP401-C-4 | 12 | Ac-SGQVPWEEPYYVVGGC |
| with GGC | | (SEQ ID NO: 12) |
| linker | | |
| HNP401-C-6 | 13 | Ac-SGQVPWEEPYYGGC |
| with GGC | | (SEQ ID NO: 13) |
| linker | | |
| HNP401-C-8 | 14 | Ac-SGQVPWEEPGGC |
| with GGC | | (SEQ ID NO: 14) |
| linker | | |
| HNP404 | 16 | DTHAHAKPRVPAFKSV |
| | | (SEQ ID NO: 16) |
| NP41 | 17 | Ac-SHSNTQTLAKAPEHTGC |
| | | (SEQ ID NO: 17) |
| HNP401-N-2 | 20 | QVPWEEPYYVVKKSS |
| | | (SEQ ID NO: 20) |
| HNP401-N-2 | 21 | QVPWEEPYYVVKKSSGG |
| (with GG | | (SEQ ID NO: 21) |
| linker) | | |
| HNP401-N-4 | 22 | PWEEPYYVVKKSS |
| | | (SEQ ID NO: 22) |
| HNP401-N-6 | 23 | EEPYYVVKKSS |
| | | (SEQ ID NO: 23) |
| HNP401-N-8 | 24 | PYYVVKKSS |
| | | (SEQ ID NO: 24) |
| HNP401-C-2 | 25 | SGQVPWEEPYYVVKK |
| | | (SEQ ID NO: 25) |
| HNP401-C-4 | 26 | SGQVPWEEPYYVV |
| | | (SEQ ID NO: 26) |
| HNP401-C-6 | 27 | SGQVPWEEPYY |
| | | (SEQ ID NO: 27) |
| HNP401-C-8 | 28 | SGQVPWEEP |
| | | (SEQ ID NO: 28) |
| HNP401-N-4 | 102 | PWEEPYYVVKKSSGG |
| (with GG | | (SEQ ID NO: 102) |
| linker) | | |
| HNP401-N-6 | 101 | EEPYYVVKKSSGG |
| (with GG | | (SEQ ID NO: 101) |
| linker) | | |
| HNP401-N-8 | 100 | PYYVVKKSSGG |
| (with GG | | (SEQ ID NO: 100) |
| linker) | | |
| HNP401-C-2 | 99 | SGQVPWEEPYYVVKKGG |
| (with GG | | (SEQ ID NO: 99) |
| linker) | | |
| HNP401-C-4 | 98 | SGQVPWEEPYYVVGG |
| (with GG | | (SEQ ID NO: 98) |
| linker) | | |
| HNP401-C-6 | 97 | SGQVPWEEPYYGG |
| (with GG | | (SEQ ID NO: 97) |
| linker) | | |
| HNP401-C-8 | 96 | SGQVPWEEPGG |
| (with GG | | (SEQ ID NO: 96) |
| linker) | | |

In embodiments. a peptide comprises, or consists of, any of SEQ ID NOS: 1-14, 16, 20-28. and 96-102.

In addition, a peptide can comprise, or consist of, any variant of the amino acid sequences described in the '054 Application. Such variants can include, without limitation, peptides comprising or consisting of amino acid sequences that share 75%-99% identity or 75%-99% homology with an amino acid sequence in Table 1 (or as otherwise disclosed in the '054 Application).

In embodiments, a peptide comprises, or consists of, an amino acid sequence having at least 75% identity to any one of SEQ ID NOS: 1-14, 16, 20-28, and 96-102. In embodiments, the peptide comprises, or consists of, an amino acid sequence having at least 80% identity to any one of SEQ ID NOS: 1-14, 16, 20-28, and 96-102. In embodiments, the peptide comprises, or consists of, an amino acid sequence having at least 85% identity to any one of SEQ ID NOS: 1-14, 16, 20-28, and 96-102. In embodiments, the peptide comprises, or consists of, an amino acid sequence having at least 90% identity to any one of SEQ ID NOS: 1-14, 16, 20-28, and 96-102. In embodiments, the peptide comprises, or consists of, an amino acid sequence having at least 95% identity to any one of SEQ ID NOS: 1-14, 16, 20-28, and 96-102. In embodiments, the peptide comprises, or consists of, an amino acid sequence having at least 97% identity to any one of SEQ ID NOS: 1-14, 16, 20-28, and 96-102.

In embodiments, a peptide consists of or comprises a peptide sequence sharing at least 80% homology with any one of SEQ ID NOS: 1-14, 16, 20-28, and 96-102. In embodiments, a peptide consists of or comprises a peptide sequence sharing at least 80% homology with any one of SEQ ID NOS: 1-14, 16, 20-28, and 96-102. In embodiments, a peptide comprises, or consists of, an amino acid sequence sharing at least 85% homology with any one of SEQ ID NOS: 1-14, 16, 20-28, and 96-102. In embodiments, a peptide comprises, or consists of, an amino acid sequence sharing at least 90% homology any one of SEQ ID NOS: 1-14, 16, 20-28, and 96-102. In embodiments, a peptide comprises, or consists of, an amino acid sequence sharing at least 95% homology with any one of SEQ ID NOS: 1-14, 16, 20-28, and 96-102. In embodiments, a peptide comprises, or consists of, an amino acid sequence sharing at least 99% homology any one of SEQ ID NOS: 1-14, 16, 20-28, and 96-102.

In embodiments, a peptide comprises, or consists of, an amino acid sequence of about 13 to about 25 amino acids comprising a core binding domain, PYYVVKK (SEQ ID NO:40), and an N-terminal sequence of QVPWEE (SEQ ID NO:41).

In embodiments, a peptide comprises an amino acid core binding domain of PYY or PYYVV (SEQ ID NO:83) and an N-terminal sequence of QVPWEE (SEQ ID NO:41). In embodiments, a peptide comprises an amino acid core binding domain of PYY and an N-terminal sequence of QVPWEE (SEQ ID NO:41). In embodiments, a peptide comprises an amino acid core binding domain of PYYVV (SEQ ID NO:83) and an N-terminal sequence of QVPWEE (SEQ ID NO:41).

In embodiments, a peptide comprises, or consists of, QVPWEEPYYVVKKSSGG (HNP401-N-2 with GG linker; SEQ ID NO:21)

In embodiments, a peptide comprises, or consists of, an amino acid sequence selected from the group consisting of: SGQVPWEEPYYVVKKSS (HNP401; SEQ ID NO:1), WEYHYVDLNWTSQHPQ (HNP402; SEQ ID NO:2), and DLPDIIWDFNWETA (HNP403; SEQ ID NO:3).

In embodiments, a peptide comprises, or consists of, an amino acid sequence selected from the group consisting of: SGQVPWEEPYYVVKKSS (HNP401; SEQ ID NO:1), Ac-QVPWEEPYYVVKKSSGGC (HNP401-N-2 with GGC linker; SEQ ID NO:7), Ac-SGQVPWEEPYYVVKKGGC (HNP401-C-2 with GGC linker; SEQ ID NO:11), QVPWEEPYYVVKKSS (HNP401-N-2; SEQ ID NO:20), QVPWEEPYYVVKKSSGG (HNP401-N-2 with GG linker; SEQ ID NO:21), and SGQVPWEEPYYVVKK (HNP401-C-2; SEQ ID NO:25), and SGQVPWEEPYYVVKKGG (HNP401-C-2 with GG linker; SEQ ID NO:99).

In embodiments, a peptide comprises, or consists of, the amino sequence of any one of SEQ ID NOS: 1, 4, 7-14, 20-28, and 96-102.

In embodiments, a peptide comprises, or consists of, the amino sequence of any one of SEQ ID NOS: 1, 20, and 22-24.

In embodiments, a peptide comprises, or consists of, the amino sequence of any one of SEQ ID NOS: 1 and 25-28.

In embodiments, a peptide comprises, or consists of, the amino sequence of any one of SEQ ID NOS: 4 and 7-10.

In embodiments, a peptide comprises, or consists of, the amino sequence of any one of SEQ ID NOS: 4 and 11-14.

In embodiments, a peptide comprises, or consists of, the amino sequence of any one of SEQ ID NOS: 21 and 100-102.

In embodiments, a peptide comprises, or consists of, the amino sequence of any one of SEQ ID NOS: 96-99.

In embodiments, a peptide is a nerve targeting peptide disclosed in PCT Application No. PCT/US21/61821 (“the '821 Application”), which is incorporated herein in its entirety. In some embodiments, these peptides were derived from phage display library screens based on selective binding to one of four different nerve target proteins: Human Myelin Protein Zero (MPZ), Myelin Basic Protein, Myelin Proteolipid Protein (Myelin PLP), and Nidogen-2. Such peptides comprise, or consist of, any of the amino acid sequences (SEQ ID NOS: 18, 19, 29-82, 84-95, and 103-471) listed in Table 2. In some embodiments, these peptides were derived from phage display screens based on selective binding to laminin trimers 421, 521, or both. Such peptides comprise, or consist of, any of the amino acid sequences listed in Table 3 (SEQ ID NOS: 472-678). In some embodiments, these peptides were derived from phage display screens based on selective binding to neuron and nerve extracts but not muscle extracts. Such peptides comprise, or consist of, any of the amino acid sequences listed in Table 4 (SEQ ID NOS: 679-746).

TABLE 2

| Nerve Target Protein | Amino Acid Sequence | SEQ ID NO: |
|---|---|---|
| R3 Human MPZ | ALPLSAS | 95 |
| | TSPWELR | 94 |
| | SHLASRP | 93 |
| | DPITRLK | 92 |
| | VLKNNPT | 91 |
| | IKPPYTH | 90 |
| | ETTRNYG | 89 |
| | SHSPGNK | 88 |
| | WPSISRP | 87 |
| | LSSPLSL | 86 |
| | VLTKPMP | 85 |
| | TMASPAK | 84 |
| | KMAGHTV | 82 |
| | SAHAKHH | 81 |
| | DRNSVFW | 80 |
| | GPLRMAT | 79 |
| | TESLFPH | 78 |
| | RVPHSRT | 77 |
| | GLTPKLF | 76 |
| | GDERIVRTLSHN | 75 |
| | CLKQQTGDC | 103 |
| | EGHVWSEYTWGT | 104 |
| | FMSDVDH | 105 |
| | ISSVTVV | 106 |
| | KQPAMFN | 107 |
| | LISPTAR | 108 |
| | LVLQTTP | 109 |
| | NPTSEGA | 110 |
| | THSTDLS | 111 |
| | TTNVTEIERESY | 112 |
| | YAPWRVF | 113 |
| | YGQSITE | 114 |
| | CDIKDANSC | 115 |
| | CIKGEDNQC | 116 |
| | HNQLLYT | 117 |
| | IEQVGWR | 118 |
| | KTPILAS | 119 |
| | LWAKRDA | 120 |
| | NPISPRN | 121 |
| | NTASMTT | 122 |
| | SAHSVVE | 123 |
| | YSSLTSN | 124 |
| | AWTMRAS | 125 |
| | CERIGPREC | 126 |
| | CQKWFTFAC | 127 |
| | CSNALNKAC | 128 |
| | FSPRDVS | 129 |
| | FSTTGRG | 130 |
| | HHPLFID | 131 |
| | LVNPFPE | 132 |
| | YEGERGA | 133 |
| | CEDRQRSMC | 134 |
| | CGEKIMSLC | 135 |
| | DRYLLNQ | 136 |
| | FPSYTIS | 137 |
| | GGSKVWG | 138 |
| | GLWRHTM | 139 |
| | KRNLWDI | 140 |
| | SPNTNSH | 141 |
| | TLLHYSA | 142 |
| | VPLAYLR | 143 |
| | VVPLKWI | 144 |
| | CRVGVTSAC | 145 |
| | FALPAGL | 146 |
| | FRVTQTA | 147 |
| | HPLMVPS | 148 |
| | LPNANNL | 149 |
| | MIHTRQT | 150 |
| | SETESYM | 151 |
| | SNALSYF | 152 |
| | SYLPPFI | 153 |
| | VHTPYRS | 154 |
| | WPRPIQI | 155 |
| | WTSVATA | 156 |
| | ALPISYL | 157 |
| | FEHMAVT | 158 |
| | NASVPPK | 159 |
| | SGHGAFR | 160 |
| | TTLVVTA | 161 |
| | CPDAARNSC | 162 |
| | CYSSVADMC | 163 |
| | LSPPRIM | 164 |
| | WMTNLDP | 165 |
| | KLPTLSV | 166 |
| | AMSNLSY | 167 |
| | HPFPRFD | 168 |
| | LPAESHW | 169 |
| | STHEWRT | 170 |
| | TTWPNTA | 171 |
| | YMKHSPG | 172 |
| | AYLEDWRSMSTR | 173 |
| | GPVVSGM | 174 |
| | HLDSRRH | 175 |
| | LVLSNPR | 176 |
| | NMSHSNR | 177 |
| | SVAYVPV | 178 |
| | YPQARTS | 179 |
| | MYHDSVS | 180 |
| | ADNVLRRALENI | 181 |
| | QIAHLEY | 182 |
| | GMVSPHHSVYRH | 183 |
| | GSWSSGF | 184 |
| | NGAYSLAIRYTS | 185 |
| | YTPNWHFRWMPA | 186 |
| | GEWRARIDQDVS | 187 |
| R3 Myelin Basic | NDTKTPS | 74 |
| Protein | HPSTWHK | 73 |
| | EHGYYKV | 72 |
| | LPMRILT | 71 |
| | SVLRMLN | 70 |
| | AITSRNA | 69 |
| | SKSDAWR | 68 |
| | ILLPSLN | 67 |
| | WTMKPNY | 66 |
| | AGQLMRCANC | 65 |
| | AWSTSSV | 64 |
| | LRPISHE | 63 |
| | MQTQGRV | 62 |
| | SPDRTSL | 61 |
| | DPWVMLR | 60 |
| | QIGVLPS | 59 |
| | QNTGLKW | 58 |
| | AGFPPNT | 57 |
| | DGKNTSN | 56 |
| | HRLPWHH | 55 |
| | AGSLLSL | 188 |
| | AIPPRKL | 189 |
| | AISWKGF | 190 |
| | AKFTMWV | 191 |
| | ALRDTRV | 192 |
| | ALYNQMT | 193 |
| | AQFLTIY | 194 |
| | AQLGAFR | 195 |
| | ARVPNPL | 196 |
| | CTMTNQYDC | 197 |
| | DSQEKSV | 198 |
| | DVRIWTL | 199 |
| | ELKSLRF | 200 |
| | ESKSPRE | 201 |
| | ETHYKIA | 202 |
| | FDLLARK | 203 |
| | FQLSYET | 204 |
| | GEPALVT | 205 |
| | GPRVTPH | 206 |
| | GQWWKML | 207 |
| | GRHTDVV | 208 |
| | GSPYGWG | 209 |
| | GVAEVGL | 210 |
| | HAHWARL | 211 |
| | HFTFFPI | 212 |
| | HPWSHPN | 213 |
| | HQMRTML | 214 |
| | HQPLFPR | 215 |
| | HRTHLHQ | 216 |
| | HSGPLLP | 217 |
| | HSSATMS | 218 |
| | IGTRWHQ | 219 |
| | INFPFAV | 220 |
| | IPIPYRT | 221 |
| | IPYSQMP | 222 |
| | KIITTKS | 223 |
| | KMSLAPP | 224 |
| | KVHHLAR | 225 |
| | LELHQNV | 226 |
| | LITHPQL | 227 |
| | LMLPQLE | 228 |
| | LVPPVNH | 229 |
| | MNSPLAQ | 230 |
| | MQRLGAD | 231 |
| | NGPNSLQ | 232 |
| | NGVSSSL | 233 |
| | NLTYLRF | 234 |
| | QGAYVRP | 235 |
| | QKALPQT | 236 |
| | QTSIMSG | 237 |
| | QYWDILG | 238 |
| | RGHPSQL | 239 |
| | RSRKHRP | 240 |
| | RTTDWWT | 241 |
| | RVIPSTT | 242 |
| | SAVLMPP | 243 |
| | SEGLTLL | 244 |
| | SGPFPLV | 245 |
| | SIQTSAM | 246 |
| | SKLAGFP | 247 |
| | SKPHASH | 248 |
| | SLDHLRL | 249 |
| | SLKTTNP | 250 |
| | SLWQTLR | 251 |
| | SLWTHRT | 252 |
| | SNAHTLP | 253 |
| | SPKPDIP | 254 |
| | SRHLPPL | 255 |
| | SSFYVAY | 256 |
| | SSMRYNS | 257 |
| | SVGLPTK | 258 |
| | SYRSMAP | 259 |
| | SYVTVFR | 260 |
| | TGPSSAV | 261 |
| | TGSVSKQ | 262 |
| | THKHMMT | 263 |
| | TIRVAHT | 264 |
| | TPGRATL | 265 |
| | TPVLWLP | 266 |
| | TPVRLNT | 267 |
| | TRIPILL | 268 |
| | TTTQGHP | 269 |
| | VGTFWTR | 270 |
| | VIRTAAM | 271 |
| | VLSPYHN | 272 |
| | VRGTPNH | 273 |
| | WDLPQNR | 274 |
| | WDLRHSK | 275 |
| | WTYHPTT | 276 |
| | WVPDRSL | 277 |
| | YPTYLSY | 278 |
| | YPYVPIY | 279 |
| | YSSRLND | 280 |
| | YTASPRW | 281 |
| | YVNRVKQ | 282 |
| | AAGQRSF | 283 |
| | AILTRPP | 284 |
| | ALGTRPL | 285 |
| | ASNTSKG | 286 |
| | AVRQTTM | 287 |
| | CEDSDKSVC | 288 |
| | CNRVPFKQC | 289 |
| | CPRMNNPLC | 290 |
| | DVVKDVI | 291 |
| | ESPYPHS | 292 |
| | FKLPWAS | 293 |
| | FLNQAPT | 294 |
| | FSAGVGK | 295 |
| | GHSHYILGKPTA | 296 |
| | GPITHAI | 297 |
| | HFLRPSD | 298 |
| | HLLKTHS | 299 |
| | HMAGVNQ | 300 |
| | HPDTLPQ | 301 |
| | HPTGTPT | 302 |
| | HTGLRSL | 303 |
| | KGQPLFR | 304 |
| | KYRHGAE | 305 |
| | LFRRVTE | 306 |
| | LTKSLNH | 307 |
| | MPLLELP | 308 |
| | MTNHGNA | 309 |
| | NLKLWTR | 310 |
| | NLLQGVM | 311 |
| | NMYPRVT | 312 |
| | NSSPHQI | 313 |
| | QGTLFDT | 314 |
| | QPIDSTS | 315 |
| | SEPGTVR | 316 |
| | SGNLKKA | 317 |
| | SIAEVLGLWRNV | 318 |
| | SIRMTEI | 319 |
| | SISKIRT | 320 |
| | SLRSVGA | 321 |
| | SSGMLSK | 322 |
| | SYTLSRS | 323 |
| | TLLTALT | 324 |
| | TPLYLSS | 325 |
| | VSFTLEP | 326 |
| | VVDMSTY | 327 |
| | WPDLRIL | 328 |
| | WSLPLLS | 329 |
| | WSPRWPS | 330 |
| | YLPPPLP | 331 |
| | DSYLLSA | 332 |
| | FVLPNKN | 333 |
| | IILPSAQ | 334 |
| | KNSIAPR | 335 |
| | LPPQISR | 336 |
| | MPSLKHQ | 337 |
| | NPTDTNK | 338 |
| | NTSLSFK | 339 |
| | QNSYLSN | 340 |
| | QQAHLQS | 341 |
| | SPPRFIP | 342 |
| | SWFESHN | 343 |
| | TPQVMLK | 344 |
| | TSPPLAH | 345 |
| | TSRLVST | 346 |
| | VAHQRVS | 347 |
| | VPTLRIP | 348 |
| | YATATPS | 349 |
| | ANLSRSV | 350 |
| | FSARTNT | 351 |
| | HFYGPGP | 352 |
| | HPWLGNR | 353 |
| | HYSPNVM | 354 |
| | NFAQHMQ | 355 |
| | SNAERWR | 356 |
| | SRPTRVP | 357 |
| | SVEYGQL | 358 |
| | TGNLRLY | 359 |
| | MDLSLKP | 360 |
| | NTPKVLA | 361 |
| | TQMTMDS | 362 |
| R3 Myelin PLP | ATLKPYR | 54 |
| | GLIRHYT | 53 |
| | MTPHTSC | 52 |
| | TNPWKPH | 51 |
| | HQGSRTY | 50 |
| | RFELPAP | 49 |
| | YTPMGTG | 48 |
| | GPFLFTV | 47 |
| | LALYARM | 46 |
| | LRFQIPP | 45 |
| | FPVVTRN | 44 |
| | RHNVEFS | 43 |
| | LTHHFAP | 42 |
| | NPSHHPR | 39 |
| | YPKLWTY | 38 |
| | FKMPIPT | 37 |
| | NPNWRTI | 36 |
| | SMWSQFR | 35 |
| | DTSRTME | 34 |
| | EKPSWTR | 33 |
| | ASLTLAL | 363 |
| | DRHEGHR | 364 |
| | FRIDKSM | 365 |
| | GHLQSLF | 366 |
| | GLWGPSL | 367 |
| | KPSASIY | 368 |
| | LPSHASI | 369 |
| | LPVKNLL | 370 |
| | MKIMPMD | 371 |
| | SAQSRSF | 372 |
| | SLPSLLP | 373 |
| | SVVYQNS | 374 |
| | TTTIADM | 375 |
| | VTGPGTP | 376 |
| | VTPNKAR | 377 |
| | WHYTPSM | 378 |
| | WSRISVD | 379 |
| | ALFQERK | 380 |
| | AQASSAR | 381 |
| | EVLYPNN | 382 |
| | HLHTRPT | 383 |
| | IRPTHNG | 384 |
| | KGVIPAT | 385 |
| | LHQTYRP | 386 |
| | LPHRLNP | 387 |
| | TGISSTP | 388 |
| | TMPIKAM | 389 |
| | VPGHISG | 390 |
| | VSASWMP | 391 |
| | WSPHGYK | 392 |
| | YSQAISA | 393 |
| | AVVAMNK | 394 |
| | GIPSSKN | 395 |
| | NDASTVS | 396 |
| | QLPRNNL | 397 |
| | SLNRGGA | 398 |
| | SLPGYRH | 399 |
| | SVLPDKL | 400 |
| | TLWAQKT | 401 |
| | TQPRYPS | 402 |
| | VFPERRV | 403 |
| | VGYRSAS | 404 |
| | VRTSMNH | 405 |
| | YTWTPSR | 406 |
| | FSLDRDG | 407 |
| | FSTPTNV | 408 |
| | GPAAVII | 409 |
| | GWSTAIR | 410 |
| | HKAPLGT | 411 |
| | IDRVRGL | 412 |
| | LMQKPSI | 413 |
| | SVMWMTP | 414 |
| | TKITPHR | 415 |
| | YSSRLTA | 416 |
| | ARFVPLT | 417 |
| | DGSRDLV | 418 |
| | EKSTTVA | 419 |
| | YSDTYKH | 420 |
| | EPSSFTF | 421 |
| | GKSYSQI | 422 |
| | GNVFQTS | 423 |
| | IIPPTSV | 424 |
| | LPLRLHA | 425 |
| | NVFSATP | 426 |
| | QRRSVIL | 427 |
| | SGVATYT | 428 |
| | SWDVLEL | 429 |
| | TFPERLR | 430 |
| | WSPHRSF | 431 |
| | IHHLESS | 432 |
| | LNGISAL | 433 |
| | SALISTR | 434 |
| | SGLHYAL | 435 |
| | TRASYPQ | 436 |
| | NIPSSIL | 437 |
| | QPNMLKP | 438 |
| | RLQPEPT | 439 |
| | SVEKYSK | 440 |
| | VSSQEQA | 441 |
| | WNTADRL | 442 |
| | MPHAPVQ | 443 |
| | TDVEMVP | 444 |
| | AHQSSVT | 445 |
| | GSAWKKH | 446 |
| R3 Nidogen-2 | ACGEGEADVC | 32 |
| | ACPENKSKHC | 31 |
| | ACEPRSLANC | 30 |
| | ACTVHKWDNC | 29 |
| | GYWRNAL | 19 |
| | KAIHPMRG | 18 |
| | CIHHKGVLC | 447 |
| | CKDGHIRHC | 448 |
| | CNLATSLQC | 449 |
| | CRSPHEPMC | 450 |
| | CVDKMASVC | 451 |
| | CYSAPTKSC | 452 |
| | EAMMHRN | 453 |
| | LHITPEV | 454 |
| | LSIRGLT | 455 |
| | NILSQVN | 456 |
| | QLLESRT | 457 |
| | QMDAKHM | 458 |
| | QTLRPKQ | 459 |
| | TIASLLV | 460 |
| | WLAAGSQ | 461 |
| | ALYLPGR | 462 |
| | ARMAFSL | 463 |
| | ATYNMSQ | 464 |
| | GLRTMEP | 465 |
| | TPWLPTI | 466 |
| | YNHTMMY | 467 |
| | CSFKMNQKC | 468 |
| | LVHPFHG | 469 |
| | CSSSAPRIC | 470 |
| | HSRLPTP | 471 |

TABLE 3

| Target Protein | Amino Acid Sequence | SEQ ID NO: |
|---|---|---|
| Laminin 421/521 | CKSPIKGTC | 472 |
| | CLQKNHKFC | 473 |
| | CVLKCEDQKYRG | 474 |
| | DGANMFNIAPAN | 475 |
| | DRMYSTVPAEGL | 476 |
| | EAVSTQI | 477 |
| | GINLRALDLHAN | 478 |
| | HQSVTGVRSHFH | 479 |
| | SVVGWAAPRTAQ | 480 |
| | TILKPAAQGFAD | 481 |
| | TSAVSLR | 482 |
| | TTLWLDRDEALK | 483 |
| | VIADQSKSAVAV | 484 |
| | AQNLRVHAWASL | 485 |
| | CPMHQSKTC | 486 |
| | CTDGRNFVC | 487 |
| | CTGETLLTC | 488 |
| | CTSSSHQTC | 489 |
| | ETGGLAYGSGQK | 490 |
| | GIVPSRHTTGLG | 491 |
| | GWTSDLSRNVRG | 492 |
| | HTQIHRL | 493 |
| | KFADTKLTSLRY | 494 |
| | QLQHVHL | 495 |
| | QPTPSQIKFTRT | 496 |
| | RALDRYLPWSPH | 497 |
| | SLPYAASLNSVE | 498 |
| | STNVQRA | 499 |
| | SVLSTGTANQRH | 500 |
| | SVQTPMWRSLVG | 501 |
| | SWHFTGTPFMNR | 502 |
| | SYTATWSEMSRS | 503 |
| | TALSHRHEAMRW | 504 |
| | TKGIAPK | 505 |
| | TVARSTAQERSI | 506 |
| | AVRPLGLPDNHR | 507 |
| | CIGGPHRNC | 508 |
| | CKSPAIKGC | 509 |
| | CNSWKAAKC | 510 |
| | DSLYRGMHQPRI | 511 |
| | EDRMMTYRYTST | 512 |
| | EVERILTPHVNN | 513 |
| | FNSDSRSTHQED | 514 |
| | HPAWADFFTMSS | 515 |
| | IDLSLRS | 516 |
| | IDQSGLQKSGMK | 517 |
| | LGSSRSPSSFLG | 518 |
| | NGHEVQSRAANR | 519 |
| | QGWKDRLPIWRY | 520 |
| | SHDVANGTSVRT | 521 |
| | SPDRTNWGWQTN | 522 |
| | SVESNSKLTMPR | 523 |
| | WPMMQTR | 524 |
| | CKSERGPEC | 525 |
| | CQGWPRPMC | 526 |
| | CSRAGLSAC | 527 |
| | CTWKHRDNC | 528 |
| | GLLDWGSLQGGN | 529 |
| | GPLLFRG | 530 |
| | GSPEKRT | 531 |
| | KASIAYD | 532 |
| | LGYGQGTPHRSN | 533 |
| | QGWEFQVPARHS | 534 |
| | QTFGYSMFNVRT | 535 |
| | RNLHYPLNHPFM | 536 |
| | TAPRWTQ | 537 |
| | VPSYILR | 538 |
| | WRSPLTT | 539 |
| | CQTSGHHQC | 540 |
| | CSSQAKRSC | 541 |
| | CTAWTRQEC | 542 |
| | EYVKFSHSPRTY | 543 |
| | GGWQSLWDKPEH | 544 |
| | GPMLKNLSDAVT | 545 |
| | NHDQGSLTRWRS | 546 |
| | SLLSVVNTSSKS | 547 |
| | VVPSWPSVHRPP | 548 |
| | WGWNGANMSPRG | 549 |
| | CHNADNNGC | 550 |
| | LPMLRHS | 551 |
| | NDTGRHASGISK | 552 |
| | QALRTNYSPLNS | 553 |
| | TLKAGTRANDGV | 554 |
| | CGETTAGKC | 555 |
| | HSTYAPG | 556 |
| | QINWKQADKNAD | 557 |
| | ALPWQSGVHGTK | 558 |
| | TFGSGRSMPIQY | 559 |
| | VYRNGGGLPLTA | 560 |
| | ALKIGPETTIYM | 561 |
| | DWTQVRVTNWFL | 562 |
| | LGHQNGGRADMW | 563 |
| | MTNSGAK | 564 |
| | WAYTDYM | 565 |
| | AHAAGRDMRQGT | 566 |
| | DVIHSSRGAYFE | 567 |
| | NWTHLGVARLQP | 568 |
| | SYDGTMLKQVRL | 569 |
| | WQWRSTELGYRY | 570 |
| | WSPANGWRHQTI | 571 |
| | CLHNGQRSC | 572 |
| | CTPRSATLC | 573 |
| | DIMKSPRSNLRS | 574 |
| | QTFHEYLNPARG | 575 |
| | SLNTTWVSPMMK | 576 |
| | AQRMNQA | 577 |
| | GDAQILM | 578 |
| | GHRTLVTSERYL | 579 |
| | LGPSTQGAGQTR | 580 |
| | TFAAAQAELIMV | 581 |
| | WEEHRVEILPDV | 582 |
| | AGAYTSRHAFDE | 583 |
| | CGLATNKSC | 584 |
| | GDVITAYINPWP | 585 |
| | GYDLSRLWGMAS | 586 |
| | ANRYLAS | 587 |
| | CKWTQLWGC | 588 |
| | CLKDTHLNC | 589 |
| | GQPRNIHLPGTH | 590 |
| | GSTTHPHFGLPG | 591 |
| | GWIQDTFVLGRS | 592 |
| | QGHPFIY | 593 |
| | SNPKHVSSLGQM | 594 |
| | WGANTTGRTHGG | 595 |
| | AQGVWWSEWFAP | 596 |
| | CFQPKMNSC | 597 |
| | CWSGNSRSC | 598 |
| | FPLKIRT | 599 |
| | LARSSIMAANNV | 600 |
| | AKTQAPSNWSGV | 601 |
| | CSVWNSGNC | 602 |
| | CTGRISKHC | 603 |
| | GAMMNSNNLVAR | 604 |
| | GGGYHIFGPLVT | 605 |
| | HSFLGAR | 606 |
| | LLAGPFR | 607 |
| | QTLQTRH | 608 |
| | ALGGEPRKAYQR | 609 |
| | CNHLTQKLC | 610 |
| | CTSKLARHC | 611 |
| | AVYPLDLGAGMR | 612 |
| | CHRTYNSTC | 613 |
| | LDRNADSVRAVL | 614 |
| | TTNGVPGHDRSP | 615 |
| | ADMPQMTLKYGV | 616 |
| | AWNGNRS | 617 |
| | GDLLLKR | 618 |
| | VPQIRIK | 619 |
| | SWQMRSY | 620 |
| | YHLGPNQKMRTS | 621 |
| | ASHSRPLMNYAP | 622 |
| | QTRWDDGSYQIS | 623 |
| | SLAVANTRFMIR | 624 |
| | EGHVWSEYTWGT | 625 |
| | ETYKVTRVISPW | 626 |
| | GHPWTEIDFMSS | 627 |
| | KQELSDNLASHR | 628 |
| | SFRNLEKLSLWS | 629 |
| | GDLWNVP | 630 |
| | GPKNHHQ | 631 |
| | GSGFRNEEHSAH | 632 |
| | NNNAAMQNHGVR | 633 |
| | RAQAHQV | 634 |
| | CSLANPATC | 635 |
| | GEPLTRFPNSDS | 636 |
| | KPILWNRSLNAL | 637 |
| | GFADVIHRKWSS | 638 |
| | HQSLAGVPWSRH | 639 |
| | VPVFLDQARVMK | 640 |
| | DDLLTAPRLGVW | 641 |
| | SDRGKTVLHGQI | 642 |
| | CGITQTTTC | 643 |
| | CHQRQQTYC | 644 |
| | EYWHQRGSWFHR | 645 |
| | YTPRNQL | 646 |
| | CTKSPSNSC | 647 |
| | WAFKAPQ | 648 |
| | GSLDMGNNKQPV | 649 |
| | AVVSQNQMSQQK | 650 |
| | CLNQKWEAC | 651 |
| | WPVRNLL | 652 |
| | GYRSFIHENWSI | 653 |
| | SGWEGKTHQGVR | 654 |
| | LQSNAADHHQGM | 655 |
| | ATSKYPNSWAQT | 656 |
| | DRLTPITWDWSR | 657 |
| | WTKHNTPAHHLS | 658 |
| | WNANYGTMTRHA | 659 |
| | HASNQRV | 660 |
| | GRTIHSM | 661 |
| | RLVTASMVSPSF | 662 |
| | DGQVSDHSYVQW | 663 |
| | KISMNRLHANFT | 664 |
| | WDLPTLRKTKQA | 665 |
| | WVAAERR | 666 |
| | MTHSAHNSQKTN | 667 |
| | WGLKLPH | 668 |
| | QGWFRHS | 669 |
| | IPVGERK | 670 |
| | CMNKIQRDC | 671 |
| | KALGYSLVGGEW | 672 |
| | APKVTNARPTQL | 673 |
| | AWVEAQNASNPS | 674 |
| | CSKHNHSRC | 675 |
| | GKPDHRLVSLWR | 676 |
| | CSHHKLKQC | 677 |
| | GGYDLPWANWQN | 678 |

TABLE 4

| Peptide | SEQ ID NO: |
|---|---|
| GWQMFPPMQNTR | 679 |
| SSLYMNGWSRDQ | 680 |
| YAGQNGWMRSDL | 681 |
| VERLNWPKRMQS | 682 |
| WDVAWLERRALH | 683 |
| VRLPMLASMGHQ | 684 |
| DRGLTILTDDLR | 685 |
| GWNEATNSARHT | 686 |
| EFARFRVFGVAE | 687 |
| NRIGHGTATGTL | 688 |
| VTDMLGIPKLHA | 689 |
| DELVGGAPWYKS | 690 |
| GLLREPDELMFL | 691 |
| QNIVENFIDNER | 692 |
| SGEYNLIKPNLL | 693 |
| EESNAKLELLSR | 694 |
| GWANSITYRNLY | 695 |
| NYAVGDRITKNI | 696 |
| HGIVFSKRPFSN | 697 |
| FDPLSRNVPHKT | 698 |
| NAVHRPDLNLLA | 699 |
| GMRTGDDSPKYP | 700 |
| IPGQTPRMPHSK | 701 |
| KTTSMTALTMGL | 702 |
| VNYQTPWNKHWY | 703 |
| VESGHAYSVSSW | 704 |
| VRSDMVSGGLQK | 705 |
| YANGIEAKDSSR | 706 |
| YAWDTVASRNYK | 707 |
| LQTHSITYRHER | 708 |
| SLGSSFVQGGEL | 709 |
| WEKHTGPLSRYL | 710 |
| AFRFEFQVPDSH | 711 |
| GHAHSLTQNPLF | 712 |
| GLTIMSSRHSLQ | 713 |
| NGMNWLQVHQLS | 714 |
| WSGGMEDNSNRL | 715 |
| AIMSYNSPWIQG | 716 |
| DVMQMFHQVHFY | 717 |
| QANLYYWDPSDL | 718 |
| QRATNDILIRGW | 719 |
| SPMANVWFNRLS | 720 |
| YSANSNPKKSLM | 721 |
| ALNHNGPVKTGL | 722 |
| DASQYPLVSGLW | 723 |
| EPWQSRDKVIEE | 724 |
| GLWDGKGRIVEV | 725 |
| GTVSYMVKMDNN | 726 |
| SLQLWNSEKVTT | 727 |
| WVGPDIIAQMRI | 728 |
| EIIVMSGSNVTN | 729 |
| HSGAYTIQASYK | 730 |
| MLKMERPGRMAY | 731 |
| SDHTFKRVMFRW | 732 |
| EMVPMKFLMMAK | 733 |
| NTEYSRYTSIWK | 734 |
| TDNGSFLGKLTS | 735 |
| TEWMVRASSIGE | 736 |
| VPVPKKMSLDPT | 737 |
| WPLKRFEQLPLK | 738 |
| MLRHVDLTEPNA | 739 |
| QSTLGVHMLRVS | 740 |
| RTVITNEMLLLV | 741 |
| SEPIEKGSRTIV | 742 |
| TLNARDQLSING | 743 |
| GTALNLQKDINK | 744 |
| SGWHLVMSMSIG | 745 |
| TSAHHDRTLAGS | 746 |

Accordingly, in embodiments, a peptide comprises, or consists of, an amino acid sequence selected from the group consisting of SEQ ID NOS: 18, 19, 29-82, 84-95, and 103-746. In embodiments, a peptide comprises, or consists of, an amino acid sequence selected from the group consisting of SEQ ID NOS: 18, 19, 29-82, 84-95, and 103-746. In embodiments, a peptide comprises, or consists of, an amino acid sequence selected from the group consisting of SEQ ID NOS: 75-82 84-95, and 103-746. In embodiments, a peptide comprises, or consists of, an amino acid sequence selected from the group consisting of SEQ ID NOS: 55-74 and 188-362. In embodiments, a peptide comprises, or consists of, an amino acid sequence selected from the group consisting of SEQ ID NOS: 75-82, 84-95, and 103-187. In embodiments, a peptide comprises, or consists of, an amino acid sequence selected from the group consisting of SEQ ID NOS: 33-54 and 363-446. In embodiments, a peptide comprises, or consists of, an amino acid sequence selected from the group consisting of SEQ ID NOS: 18, 19, 29-32, and 447-471. In embodiments, a peptide comprises, or consists of, an amino acid sequence selected from the group consisting of SEQ ID NOS: 472-678. In embodiments, a peptide comprises, or consists of, an amino acid sequence selected from the group consisting of SEQ ID NOS: 679-746.

In embodiments, a peptide comprises, or consists of, a variant of an amino acid sequence described in International Patent Application No. PCT/US21/61821 (“the '821 Application”). Such variants therefore include, without limitation, peptides comprising or consisting of amino acid sequences that share 75%-99% identity or 75%-99% homology with any one of SEQ ID NOS: 18, 19, 29-82, 84-95, and 103-746.

In embodiments, a peptide comprises, or consists of, an amino acid sequence having at least 75% identity to any one of SEQ ID NOS: 18, 19, 29-82, 84-95, and 103-746. In embodiments, the peptide comprises, or consists of, an amino acid sequence having at least 80% identity to any one of SEQ ID NOS: 18, 19, 29-82, 84-95, and 103-746. In embodiments, the peptide comprises, or consists of, an amino acid sequence having at least 85% identity to any one of SEQ ID NOS: 18, 19, 29-82, 84-95, and 103-746. In embodiments, the peptide comprises, or consists of, an amino acid sequence having at least 90% identity to any one of SEQ ID NOS: 18, 19, 29-82, 84-95, and 103-746. In embodiments, the peptide comprises, or consists of, an amino acid sequence having at least 95% identity to any one of SEQ ID NOS: 18, 19, 29-82, 84-95, and 103-746.

In embodiments, a peptide consists of or comprises an amino acid sequence sharing at least 80% homology with any one of SEQ ID NOS: 18, 19, 29-82, 84-95, and 103-746. In embodiments, a peptide comprises, or consists of, an amino acid sequence sharing at least 85% homology with any one of SEQ ID NOS: 18, 19, 29-82, 84-95, and 103-746. In embodiments, a peptide comprises, or consists of, an amino acid sequence sharing at least 90% homology any one of SEQ ID NOS: 18, 19, 29-82, 84-95, and 103-746. In embodiments, a peptide comprises, or consists of, an amino acid sequence sharing at least 95% homology with any one of SEQ ID NOS: 18, 19, 29-82, 84-95, and 103-746. In embodiments, a peptide comprises, or consists of, an amino acid sequence sharing at least 99% homology any one of SEQ ID NOS: 18, 19, 29-82, 84-95, and 103-746.

The peptides of the present invention are synthesized by any suitable method known to one of skill in the art, including those disclosed in the '054 and '821 Applications, as incorporated herein. For examples, peptides of the present invention can be chemically synthesized by solid phase peptide synthesis. In embodiments, peptides of the present invention are acetylated at the N-terminus (“Ac” or “acetyl”), amidated at the C-terminus (“CONH₂” or “NH₂”), or both. For example, SEQ ID NO:747 corresponds to an amidated peptide, terminating with a glycinamide.

### Fluorescent Moieties

As used herein, the term “fluorescent moiety” means any fluorescent molecule, including a fluorescent protein, a fluorescent peptide, a fluorophore (such as a fluorescent dye), or any other fluorescent ligand or marker.

Specific examples of fluorescent moieties described herein are illustrative only and not meant to limit the fluorescent moieties for use with the peptides disclosed herein.

Fluorophores (or fluorescent dyes) include molecules that function in the visible, as well as near infrared (NIR) region.

Common classes of fluorophores include, but are not limited to, xanthenes, such as rhodamines, rhodols and fluoresceins, and their derivatives; bimanes; coumarins and their derivatives such as umbelliferone and aminomethyl coumarins; aromatic amines such as dansyl; squarate dyes; benzofurans; fluorescent cyanines; carbazoles; dicyanomethylene pyranes; polymethines; oxabenzanthrane; pyrylium; carbostyl; perylene; acridone; quinacridone; rubrene; anthracene; coronene; phenanthrecene; pyrene; butadiene; stilbene; porphyrin; pthalocyanine; lanthanide metal chelate complexes; rare-earth metal chelate complexes; and derivatives thereof. Fluorescent dyes are discussed, for example, in U.S. Pat. Nos. 4,452,720; 5,227,487; and 5,543,295.

Typical fluorescein dyes for use with the conjugates disclosed herein include, but are not limited to, carboxyfluorescein, 5-carboxyfluorescein, 6-carboxyfluorescein, 5(6)-carboxyfluorescein, fluorescein isothiocyanate, fluorescein-5-isothiocyanate, fluorescein-6-isothiocyanate, 5,6-dicarboxyfluorescein, 5-(and 6)-sulfofluorescein, sulfonefluorescein, succinyl fluorescein, 5-(and 6)-carboxy SNARF-1, carboxyfluorescein sulfonate, carboxyfluorescein zwitterion, carboxyfluorescein quaternary ammonium, carboxyfluorescein phosphonate, carboxyfluorescein GABA, carboxyfluorescein-cys-Cy5, and fluorescein glutathione. Examples of fluorescein dyes can also be found, for example, in U.S. Pat. Nos. 6,008,379, 5,750,409, 5,066,580, and 4,439,356.

In embodiments, the fluorescent moiety comprises a carboxyfluorescein molecule (FAM), namely a fluorescein molecule with an added carboxyl group. The term “FAM” can therefore include a fluorescein molecule with a carboxyl group in the 5-position (5-FAM) or in the 6-position (6-FAM).

In embodiments, a fluorescent moiety can include IR800CW, cyan fluorescent protein (CFP); EGFP; 5-FAM; 6-FAM; FAM; fluorescein; IAEDANS; EDANS; BODIPY FL; TRITC; Cy5; Cy3; YFP; LC Red 640; Alexa Fluor 546; tetramethylrhodamine; Dabcyl; QSY 7; QSY 9; QSY 21; and BBQ-650 dyes.

In embodiments, a fluorescent moiety comprises a rhodamine dye, such as, for example, 5-(and 6)-carboxy rhodamine 110, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride (TEXAS RED), and other rhodamine dyes. Other rhodamine dyes can be found, for example, in U.S. Pat. Nos. 6,080,852; 6,025,505; 5,936,087; 5,750,409.

In embodiments, a fluorescent moiety includes a cyanine dye, such as, for example, Cy2, Cy3, Cy3B, Cy3.5, Cy5, Cy5.5, Cy 7, and indocyanine green.

In embodiments, the fluorophore exhibits green fluorescence (for example 494 nm/519 nm or 494/524 nm), orange fluorescence (for example 554 nm/570 nm), red fluorescence (for example 590 nm/617 nm), or far red fluorescence (for example 651 nm/672 nm) excitation/emission spectra. In embodiments, the fluorophore is a fluorophore with excitation and emission spectra in the range of about 350 nm to about 775 nm. In embodiments the excitation and emission spectra are about 346 nm/446 nm, about 494 nm/519 nm, about 554 nm/570 nm, about 555 nm/572 nm, about 590 nm/617 nm, about 651 nm/672 nm, about 679 nm/702 nm or about 749 nm/775 nm.

In embodiments, the excitation and emission spectrum of the fluorophore are in the visible spectral region (about 380 nm to about 700 nm).

In embodiments, the excitation and emission spectrum of the fluorophore are in the near infrared (NIR) range, including the NIR-I spectral region (about 700 nm to about 900 nm) and NIR-II spectral region (about 1000 nm to about 1700 nm). Sec, e.g., Zhu et al. 2018, Theranostics 8, 4141-4151.

In embodiments, the fluorophore can include but is not limited to AlexaFluor 3, AlexaFluor 5, AlexaFluor 350, AlexaFluor 405, AlexaFluor 430, AlexaFluor 488, AlexaFluor 500, AlexaFluor 514, AlexaFluor 532, AlexaFluor 546, AlexaFluor 555, AlexaFluor 568, AlexaFluor 594, AlexaFluor 610, AlexaFluor 633, AlexaFluor 647, AlexaFluor 660, AlexaFluor 680, AlexaFluor 700, and AlexaFluor 750 (Molecular Probes AlexaFluor dyes, available from Life Technologies, Inc. (USA)). In embodiments, the fluorophore can include but is not limited to Cy dyes, including Cy2, Cy3, Cy3B, Cy3.5, Cy5, Cy5.5 and Cy7 (available from GE Life Sciences or Lumiprobes). In embodiments the fluorophore can include but is not limited to DyLight 350, DyLight 405, DyLight 488, DyLight 550, DyLight 594, DyLight 633, DyLight 650, DyLight 680, DyLight 750 and DyLight 800 (available from Thermo Scientific (USA)). In embodiments, the fluorophore can include but is not limited to a FluoProbes 390, FluoProbes 488, FluoProbes 532, FluoProbes 547H, FluoProbes 594, FluoProbes 647H, FluoProbes 682, FluoProbes 752 and FluoProbes 782, AMCA, DEAC (7-Diethylaminocoumarin-3-carboxylic acid); 7-Hydroxy-4-methylcoumarin-3; 7-Hydroxycoumarin-3; MCA (7-Methoxycoumarin-4-acetic acid); 7-Methoxycoumarin-3; AMF (4′-(Aminomethyl) fluorescein); 5-DTAF (5-(4,6-Dichlorotriazinyl)aminofluorescein); 6-DTAF (6-(4,6-Dichlorotriazinyl) aminofluorescein); FAM (Carboxyfluorescein); 6-FAM (6-Carboxyfluorescein), 5 (6)-FAM cadaverine; 5-FAM cadaverine; 5(6)-FAM ethylenediamme; 5-FAM ethylenediamme; 5-FITC (FITC Isomer I; fluorescein-5-isothiocyanate); 5-FITC cadaverin; Fluorescein-5-maleimide; 5-IAF (5-Iodoacetamidofluorescein); 6-JOE (6-Carboxy-4′,5′-dichloro-2′,7′-dimethoxyfluorescein); 5-CR1 10 (5-Carboxyrhodamine 110); 6-CR1 10 (6-Carboxyrhodamine 110); 5-CR6G (5-Carboxyrhodamine 6G); 6-CR6G (6-Carboxyrhodamine 6G); 5 (6)-Carboxyrhodamine 6G cadaverine; 5 (6)-Caroxyrhodamine 6G ethylenediamme; 5-ROX (5-Carboxy-X-rhodamine); 6-ROX (6-Carboxy-X-rhodamine); 5-TAMRA (5-Carboxytetramethylrhodamine); 6-TAMRA (6-Carboxytetramethylrhodamine); 5-TAMRA cadaverine; 6-TAMRA cadaverine; 5-TAMRA ethylenediamme; 6-TAMRA ethylenediamme; 5-TMR C6 maleimide; 6-TMR C6 maleimide; TR C2 maleimide; TR cadaverine; 5-TRITC; G isomer (Tetramethylrhodamine-5-isothiocyanate); 6-TRITC; R isomer (Tetramethylrhodamine-6-isothiocyanate); Dansyl cadaverine (5-Dimethylaminonaphthalene-1-(N-(5-aminopentyl))sulfonamide); EDANS C2 maleimide; fluorescamine; NBD; and pyrromethene and derivatives thereof.

In embodiments, a fluorescent moiety comprises an environmentally sensitive fluorescent dye or fluorophore. Examples of environmentally sensitive fluorescent dyes or fluorophores include 5,6-carboxy-diethyl rhodol (pH sensitive), merocyanine (membrane potential sensitive), and Nile red carboxylic acid (lipid sensitive).

In embodiments, the fluorescent moiety is a fluorescent peptide or fluorescent protein. In embodiments, the fluorescent protein is Green Fluorescent Protein (GFP). In embodiments, the fluorescent moiety is a derivative or variant of GFP, including for example: EGFP, emerald, superfolder GFP, azami green mWasabi, TagGFP, TurboGFP, AcGFP, ZsGreen, T-Sapphire, EBFP, EBFP2, Azurite, mTagBFP, ECFP, mECFP, Cerulean, mTurquoise, CyPet, AmCyan1, Midori-Ishi Cyan, TagCFP, mTFPI (Teal), EYFP, Topaz, Venus, mCitrine, YPet, TagYFP, PhiYFP, ZsYellow1, mBanana, Kusabira Orange, Kusbira Orange2, mOrange, mOrange2, dTomato, dTomato-Tandem, TagRFP, TagRFP-Y, DsRed, DsRed2, DsRed-Express (T1), DsRed-Monomer, mTangerine, mRuby, mApple, mStrawberry, AsRed2, mRFP1, JRed, mCherry, HcRed1, mRaspberry, dKeima-Tandem, HcRed-Tandem, mPlum, AQ143, mKalamal, YFP, and Citrine.

In embodiments, the fluorescent moiety includes a fluorescent molecule, as disclosed in the '054 and '821 Applications, which are incorporated in their entireties herein.

In embodiments, the fluorescent moiety is conjugated to high molecular weight molecule, such as water soluble polymers including, but not limited to, dextran, PEG, serum albumin, or poly (amidoamine) dendrimer.

A fluorescent moiety disclosed herein may be bound to the N-terminus, C-terminus, or an internal position (e.g., to an internal amino acid) of the peptide. In embodiments, two, three, four or more peptides are directly or indirectly bound to a fluorescent moiety. In embodiments, any peptide disclosed herein is bound to two or more fluorescent moieties, which may be the same or different.

In embodiments, a fluorescent moiety disclosed herein is bound directly to any peptide disclosed herein.

### Linkers

In embodiments, the fluorescent moiety is bound indirectly to the peptide, e.g., via a linker. As used herein, a “linker” is any molecule capable of binding (e.g., covalently) to a peptide disclosed herein. Linkers include, but are not limited to, straight or branched-chain carbon-containing linkers, heterocyclic carbon-containing linkers, amino acid linkers (e.g., D- or L-amino acid), lipophilic residues, peptide linkers, peptide nucleic acid linkers, hydrazone linkers, SPDB disulfide, sulfo-SPDB, maleimidomethyl cyclohexane-1-carboxylate (MCC), aminohexanoic acid linkers, and polyether linkers (e.g., PEG). Linkers include, for example, poly(ethylene glycol) linkers available from Quanta Biodesign, Powell, OH. These linkers optionally have amide linkages, sulfhydryl linkages, or hetero functional linkages. Linkers also include other linker molecules as disclosed or described in the art, including those described in the '054 and '821 Applications, as incorporated herein.

In embodiments, the linker binds to a targeting molecule disclosed herein by a covalent linkage. A linker may connect a cargo molecule, such as a fluorescent moiety, to a peptide by forming a covalent linkage to the cargo molecule at one location and a covalent linkage to the peptide at another location. The covalent linkages can be formed by reaction between functional groups on the linker and functional groups on the peptide and on the cargo molecule. In embodiments, the covalent linkage comprises an ether bond, thioether bond, amine bond, amide bond, carbon-carbon bond, carbon-nitrogen bond, carbon-oxygen bond, or carbon-sulfur bond. For example, a covalent linkage can be formed by a condensation reaction between a carboxylic acid functional group at the 5-position of fluorescein and the primary amine at the N-terminus of a peptide, such as in the fluorescent conjugate 5-FAM-QVPWEEPYYVVKKSSGG-NH2 (HNP401-N-2 with GG linker; SEQ ID NO:747).

In embodiments, the linker is flexible. In embodiments, the linker is rigid. In embodiments, the linker has segment(s) of flexibility and segment(s) of rigidity.

In embodiments, the linker comprises a linear structure. In embodiments, the linker comprises a non-linear structure. In embodiments, the linker comprises a branched structure. In embodiments, the linker comprises a cyclic structure.

In embodiments, the linker is an alkyl. In embodiments, the linker is heteroalkyl.

In embodiments, the linker is an alkylene. In embodiments, the linker is an alkenylene. In embodiments, the linker is an alkynylene. In embodiments, the linker is a heteroalkylene.

An “alkyl” group refers to an aliphatic hydrocarbon group. The alkyl moiety may be a saturated alkyl or an unsaturated alkyl. Depending on the structure, an alkyl group can be a monoradical or a diradical (i.e., an alkylene group).

The “alkyl” moiety may have 1 to 10 carbon atoms (whenever it appears herein, a numerical range such as “1 to 10” refers to each integer in the given range; e.g., “1 to 10 carbon atoms” means that the alkyl group may consist of 1 carbon atom, 2 carbon atoms, 3 carbon atoms, etc., up to and including 10 carbon atoms, although the present definition also covers the occurrence of the term “alkyl” where no numerical range is designated). The alkyl group could also be a “lower alkyl” having 1 to 6 carbon atoms. The alkyl group of the compounds described herein may be designated as “Ci-C4 alkyl” or similar designations. By way of example only, “C1-C4 alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, ethenyl, propenyl, butenyl, and the like.

In embodiments, the linker comprises a ring structure (e.g., an aryl). As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). Rings can be optionally substituted. Rings can be monocyclic or polycyclic.

As used herein, the term “aryl” refers to an aromatic ring wherein each of the atoms forming the ring is a carbon atom. Aryl rings can be formed by five, six, seven, eight, nine, or more than nine carbon atoms. Aryl groups can be optionally substituted. Examples of aryl groups include, but are not limited to phenyl, naphthalenyl, phenanthrenyl, anthracenyl, fluorenyl, and indenyl. Depending on the structure, an aryl group can be a monoradical or a diradical (i.e., an arylene group).

The term “cycloalkyl” refers to a monocyclic or polycyclic non-aromatic radical, wherein each of the atoms forming the ring (i.e. skeletal atoms) is a carbon atom. Cycloalkyls may be saturated, or partially unsaturated. Cycloalkyl groups include groups having from 3 to 10 ring atoms. Cycloalkyls include, but are not limited to, cyclopropyl, cyclobutyl, cyclopentyl, cyclohexyl, cycloheptyl, and cyclooctyl.

In embodiments, the ring is a cycloalkane. In embodiments, the ring is a cycloalkene.

In embodiments, the ring is an aromatic ring. The term “aromatic” refers to a planar ring having a delocalized x-electron system containing 4n+2π electrons, where n is an integer. Aromatic rings can be formed from five, six, seven, eight, nine, or more than nine atoms. Aromatics can be optionally substituted. The term “aromatic” includes both carbocyclic aryl (e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

In embodiments, the ring is a heterocycle. The term “heterocycle” refers to heteroaromatic and heteroalicyclic groups containing one to four heteroatoms each selected from O, S and N, wherein cach heterocyclic group has from 4 to 10 atoms in its ring system, and with the proviso that the ring of said group does not contain two adjacent O or S atoms. Non-aromatic heterocyclic groups include groups having only 3 atoms in their ring system, but aromatic heterocyclic groups must have at least 5 atoms in their ring system. The heterocyclic groups include benzo-fused ring systems. An example of a 3-membered heterocyclic group is aziridinyl. An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thictanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and faropyridinyl. The foregoing groups, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl or imidazol-3-yl (both N-attached) or imidazol-2-yl, imidazol-4-yl or imidazol-5-yl (all C-attached). The heterocyclic groups include benzo-fused ring systems and ring systems substituted with one or two oxo (=0) moieties such as pyrrolidin-2-one. Depending on the structure, a heterocycle group can be a monoradical or a diradical (i.e., a heterocyclene group).

In embodiments, the ring is fused. The term “fused” refers to structures in which two or more rings share one or more bonds. In embodiments, the ring is a dimer. In embodiments, the ring is a trimer. In embodiments, the ring is a substituted.

The term “carbocyclic” or “carbocycle” refers to a ring wherein each of the atoms forming the ring is a carbon atom. Carbocycle includes aryl and cycloalkyl. The term thus distinguishes carbocycle from heterocycle (“heterocyclic”) in which the ring backbone contains at least one atom which is different from carbon (i.e., a heteroatom). Heterocycle includes heteroaryl and heterocycloalkyl. Carbocycles and heterocycles can be optionally substituted.

In embodiments, the linker is substituted. The term “optionally substituted” or “substituted” means that the referenced group may be substituted with one or more additional group(s) individually and independently selected from Ci-Cealkyl, C3-Cgcycloalkyl, aryl, heteroaryl, C2-C6heteroalicyclic, hydroxy, Ci-C6alkoxy, aryloxy, Ci-C6alkylthio, arylthio, Ci-C6alkylsulfoxide, arylsulfoxide, Ci-C6alkylsulfone, arylsulfone, cyano, halo, C2-C8acyl, C2-C8acyloxy, nitro, Ci-C6haloalkyl, Ci-C6fluoroalkyl, and amino, including Ci-C6alkylamino, and the protected derivatives thereof. By way of example, an optional substituents may be LSRS, wherein each Ls is independently selected from a bond, —O—, —C(═O)—, —S—, —S(═O)—, —S(O)2-, —NH—, —NHC(O)—, —C(O)NH—, S(O) 2NH—, —NHS(O)2-, —OC(O) NH—, —NHC(O)O—, —(CpC6alkyl)-, or —(C2-C6alkenyl)-; and each Rs is independently selected from H, (Ci-C4alkyl), (C3-C8cycloalkyl), heteroaryl, aryl, and Ci-C6heteroalkyl. Optionally substituted non-aromatic groups may be substituted with one or more oxo (=0). The protecting groups that may form the protective derivatives of the above substituents are known to those of skill in the art.

In embodiments, a bifunctional linker having one functional group reactive with a group on one molecule (e.g., a targeting molecule), and another group reactive on the other molecule (e.g., a fluorescent moiety or a drug), is used to form the desired conjugate. Alternatively, derivatization is performed to provide functional groups. Thus, for example, procedures for the generation of free sulfhydryl groups on peptides are also known (See U.S. Pat. No. 4,659,839). A linker may alternatively comprise a heterobifunctional crosslinker comprising two or more different reactive groups that form a heterocyclic ring that can interact with a targeting molecule. For example, a heterobifunctional crosslinker such as cysteine may comprise an amine reactive group and a thiol-reactive group can interact with an aldehyde on a derivatized targeting molecule. Additional combinations of reactive groups suitable for heterobifunctional crosslinkers include, for example, amine-and sulfhydryl reactive groups; carbonyl and sulfhydryl reactive groups; amine and photoreactive groups; sulfhydryl and photoreactive groups; carbonyl and photoreactive groups; carboxylate and photoreactive groups; and arginine and photoreactive groups. Examples of a heterobifunctional crosslinker include N-Succinimidyl 4-(2-pyridyldithio) butanoate (SPDB) and maleimidomethyl cyclohexane-1-carboxylate (MCC).

In embodiments, a linker is cleavable. In embodiments, the linker is non-cleavable. A linker can be chemically stable to extracellular environments, for example, chemically stable in the blood stream, and/or may include linkages that are not stable. A linker can include linkages that are designed to cleave and/or immolate or otherwise breakdown specifically or non-specifically inside cells. A cleavable linker can be sensitive to enzymes at a specific site, such as by extracellular proteases.

A cleavable linker can include a valine-citrulline peptide, a valine-alanine peptide, a phenylalanine-lysine or other peptide, such as a peptide that forms a protease recognition and cleavage site. Such a peptide-containing linker can contain a pentafluorophenyl group. A peptide-containing linker can include a succimide or a maleimide group. A peptide-containing linker can include a para aminobenzoic acid (PABA) group. A peptide-containing linker can include an aminobenzyloxycarbonyl (PABC) group. A peptide-containing linker can include a PABA or PABC group and a pentafluorophenyl group. A peptide-containing linker can include a PABA or PABC group and a succinimide group. A peptide-containing linker can include a PABA or PABC group and a malcimide group.

A non-cleavable linker is generally protease-insensitive and insensitive to intracellular processes. A non-cleavable linker can include a maleimide group. A non-cleavable linker can include a succinimide group. A non-cleavable linker can be maleimido-alkyl-C(O)-linker. A non-cleavable linker can be maleimidocaproyl linker. A maleimidocaproyl linker can be N-malcimidomethylcyclohexane-1-carboxylate. A maleimidocaproyl linker can include a succinimide group. A maleimidocaproyl linker can include pentafluorophenyl group.

In embodiments, a peptide linker consisting of one or more amino acids is used to join the targeting molecule and a fluorescent moiety or drug. Generally the peptide linker will have no specific biological activity other than to join the molecules or to preserve some minimum distance or other spatial relationship between them. However, the constituent amino acids of the linker may be selected to influence some property of the molecule such as the folding, net charge, or hydrophobicity. In embodiments the peptide linker is relatively short, typically less than about 10 amino acids, preferably less than about 8 amino acids and more preferably less than 5 amino acids. Non-limiting illustrative examples include glycine and glycine-serine linkers which can be added to the C-terminus of a peptide. In embodiments, a peptide linker comprises a cysteine residue or an unnatural amino acid residue, e.g., selenocysteine (Sec), p-acetophenylalanine (pAcF), p-azidomethyl-L-phenylalanine (pAMF), and azido-lysine (AzK)) for site specific conjugation. In embodiments, a peptide linker is a glycine-glycine-glycine-cysteine (GGGC) (SEQ ID NO:15) linker, a glycine-glycine-cysteine (GGC) linker, a glycine-glycine (GG) linker, or a cysteine (C) linker. In embodiments, the GGGC (SEQ ID NO:15), GGC, GG, or C linker is added to the N-terminus or C-terminus of a peptide.

In embodiments, the fluorescent conjugates of the present invention may optionally be conjugated to high molecular weight molecules that increase their multivalency and avidity of binding. In embodiments, the high molecular weight molecules are water-soluble polymers. Examples of suitable water-soluble polymers include, but are not limited to, peptides, saccharides, poly (vinyls), poly (ethers), poly (amines), poly (carboxylic acids) and the like. In embodiments, the water-soluble polymers is dextran, polyethylene glycol (PEG), polyoxyalkylene, polysialic acid, starch, or hydroxyethyl starch. Any suitable method is used to conjugate peptides to water-soluble polymers (see, Hermanson G., Bioconjugate Techniques 2nd Ed., Academic Press, Inc. 2008).

In embodiments, the fluorescent conjugates of the present invention may be modified to increase solubility. Peptide modifications that increase solubility include addition of hydrophilic amino acid(s), a PEG moiety, or both. In embodiments, a PEG moiety is 8-Amino-3,6-dioxaoctanoic acid (AEEA); 12-amino-4,7,10-trioxadodecanoic acid; or 15-amino-4,7,10,13-tetraoxapenta-decanoic acid. In embodiments, approximately one to ten (e.g., one, two, three, four, five, six, seven, eight, nine, or ten) hydrophilic amino acids may be added to the N-terminus, C-terminus, an internal position, or any combination thereof, of the peptide to increase solubility. Hydrophilic amino acids include D, E, H, K, N, Q, R, S, T, and G. In embodiments, the peptide comprises a K, KK, G, or GG at the N-terminus or C-terminus.

In embodiments, the fluorescent conjugate comprises 5-FAM-QVPWEEPYYVVKKSSGG-NH2 (HNP401-N-2 with GG linker; SEQ ID NO:747) and can be used in any of the methods described herein.

In embodiments, the fluorescent conjugate comprises FAM linked to the C-terminal cysteine of HNP401 with GCC linker (Ac-SGQVPWEEPYYVVKKSSGGC-CONH₂: SEQ ID NO:748).

### IV. Pharmaceutical Compositions

Disclosed herein, in certain embodiments, are pharmaceutical compositions comprising a fluorescent conjugate disclosed herein. Pharmaceutical compositions herein are formulated using one or more physiologically acceptable carriers, including excipients and auxiliaries, which facilitate processing of the active agents into preparations which are used pharmaceutically. Proper formulation is dependent upon the route of administration chosen. Such compositions can be prepared with methods and excipients known in the art, as found, for example, in Gennaro: Remington: The Science and Practice of Pharmacy, 21^{st} Ed. (Lippincott Williams & Wilkins, 2005); Allen: Ansel's Pharmaceutical Dosage Forms and Drug Delivery Systems, 11th Ed. (Wolters Kluwer, 2018); Brunton et al.: Goodman & Gilman's The Pharmacological Basis of Therapeutics (McGraw-Hill Professional, 2005); and Rowe: Handbook of Pharmaceutical Excipients (Pharmaceutical Press, 2005).

In embodiments of pharmaceutical compositions, a peptide in a fluorescent conjugate can consist of, or comprise, any amino acid sequence, including all variants and combinations, described herein. In embodiments, a peptide consists of, or comprises. any amino acid sequence listed in Table 1, or any variant thereof, as described herein. In embodiments, a peptide comprises, or consists of, any of SEQ ID NOS: 1-14, 16, 20-28, and 96-102. In embodiments, the peptide consist of, or comprises, any amino acid sequence listed in Tables 2-4, or any variant thereof. In embodiments, a peptide comprises an amino acid sequence selected from SEQ NOS: 75-82 84-95, and 103-187. In embodiments, a peptide comprises an amino acid sequence selected from SEQ NOS: 55-74 and 188-362. In embodiments, a peptide comprises an amino acid sequence selected from SEQ NOS: 33-54 and 363-446. In embodiments, a peptide comprises an amino acid sequence selected from SEQ NOS: 18, 19, and 29-32, and 447-471. In embodiments, a peptide comprises an amino acid sequence selected from SEQ ID NOS: 472-678. In embodiments, a peptide comprises an amino acid sequence selected from SEQ ID NOS: 679-746. In embodiments, a peptide consists or, or comprises, the amino acid sequence of SEQ ID NO:21.

In embodiments, the fluorescent conjugate in a pharmaceutical composition comprises SEQ ID NO:747.

In embodiments, a pharmaceutical composition disclosed herein further comprises a pharmaceutically acceptable diluent, excipient, carrier, or solvent. Examples of suitable aqueous and non-aqueous carriers, diluents, solvents, or vehicles include water, ethanol, polyols (propylene glycol, polyethylene-glycol, glycerol, cremophor and the like), suitable mixtures thereof, vegetable oils (such as olive oil) and injectable organic esters such as ethyl oleate. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. Bioavailability enhancers may include penetration or permeation enhancers. See, e.g., Muheem et al., 2016, Saudi Pharm. J. 24, 413-428; Brayden et al. 2020, Adv. Drug Deliv. Rev. 2020 May 29:S0169-409X(20) 30040-5; Ibrahim et al. 2020, J. Pham. Sci. 28, 403-416. In some formulations, proper fluidity can be maintained, for example, by the use of a coating such as lecithin, by the maintenance of the required particle size in the case of dispersions, and by the use of surfactants.

In embodiments, the pharmaceutical compositions include other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions also contain other therapeutically valuable substances.

In embodiments, peptide conjugates disclosed herein are delivered to a subject via a drug delivery vehicle or carrier. In embodiments, a delivery vehicle is made from natural or synthetic materials or both. In embodiments, a delivery vehicle is a nanoparticle, microparticle, polymeric micelle, nanocapsule, dendrimer, large PEG, nanogel, liposome, fullerene, nanostructured lipid carrier, nanoshell, quantum dot, protein-based nanocarriers (e.g., albumin, elastin, gliadin, legumin, zein, soy protein, milk protein, whey based nanocarriers), organic nanocarrier (e.g., gelatin, dextran, guar gum, chitosan, collagen), polysaccharide based carrier (e.g., dextran, chitosan, pectin), lipid emulsion, or a combination thereof.

In embodiments, a pharmaceutical composition disclosed herein is administered to a subject by any suitable administration route, including but not limited to, topical, oral, intrarectal, intravaginal, intranasal, inhalation, parenteral (intravenous, intraarterial, subcutaneous, intraperitoneal, intramuscular, intravascular, intrathecal, intravitreal, infusion) administration. In certain embodiments, a pharmaceutical composition disclosed herein is administered to a subject is administered locally or systemically. In embodiments, a pharmaceutical composition disclosed herein is administered intravenously.

Formulations suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Formulations suitable for subcutaneous injection also contain optional additives such as preserving, wetting, emulsifying, and dispensing agents, as are known in the art. In embodiments, formulations suitable for intravenous injection may be prepared in aqueous solutions, such as saline buffers and other physiologically compatible buffers known in the art. For intravenous injections, an active agent is optionally formulated in aqueous solutions, preferably in physiologically compatible buffers such as Hank's solution, Ringer's solution, or physiological saline buffer.

Parenteral injections optionally involve bolus injection or continuous infusion. Formulations for injection are optionally presented in unit dosage form, e.g., in ampoules or in multi dose containers, with an added preservative. In embodiments, the pharmaceutical composition described herein are in a form suitable for parenteral injection as sterile suspensions, solutions or emulsions in oily or aqueous vehicles, and contain formulation agents such as suspending, stabilizing and/or dispersing agents. Pharmaceutical formulations for parenteral administration include aqueous solutions of an active agent in water soluble form. Additionally, suspensions are optionally prepared as appropriate oily injection suspensions.

In embodiments, a pharmaceutical composition herein is administered orally. Dosage forms suitable for oral administration can be solid or liquid and may include for example, a pill, capsule, troche, tablet, caplet, gel caplet (gel cap), syrup, an aqueous suspension or solution, a chewable form, a swallowable form, a dissolvable form, an effervescent, a granulated form, and an oral liquid solution. In a specific embodiment, the dosage form is a solid dosage form, and more specifically, comprises a tablet or capsule, or is orally administered by a vehicle or carrier as disclosed herein.

In embodiments, a pharmaceutical composition described herein is in a unit dosage form suitable for single administration of precise dosages. In unit dosage form, the formulation is divided into unit doses containing appropriate quantities of an active agent disclosed herein. In embodiments, the unit dosage is in the form of a package containing discrete quantities of the formulation. Non-limiting examples are packaged tablets or capsules, and powders in vials or ampoules. In embodiments, aqueous suspension compositions are packaged in single-dose non-reclosable containers. Alternatively, multiple-dose reclosable containers are used, in which case it is typical to include a preservative in the composition. By way of example only, formulations for parenteral injection are presented in unit dosage form, which include, but are not limited to ampoules, or in multi dose containers, with an added preservative.

In embodiments, a pharmaceutical composition is administered via systemic intravenous to subjects, particularly, human patients, and more particularly, human patients undergoing surgery.

In embodiments, a pharmaceutical composition is administered orally to subjects, particularly, human patients, and more particularly, human patients undergoing surgery.

### EXAMPLE 1

### Use of Nerve Targeting Fluorescent Conjugates in Ureter Visualization

### Introduction

Phage display screens were previously carried out to identify peptides that bind to human nerve homogenates or to isolated human nerve proteins. See the '054 and '821 Applications. Such peptides can be linked or tagged to cargo molecules for various applications. If tagged with a fluorescent moiety, for example, such conjugates are potentially useful for in vivo labeling of nerves during fluorescence assisted surgery, and if linked to an active agent, for example, such conjugates are potentially useful as targeted therapies.

Exemplary peptides were conjugated to fluorescent dyes to generate fluorescent conjugates (also referred to here as conjugates) and evaluated in rodent models, where they showed sensitive nerve detection following intravenous administration. These conjugates included FAM-NP41, which comprises the fluorescent dye, carboxyfluorescein (FAM), coupled to the C-terminal lysine of acetylated NP41 (Ac-SHSNTQTLAKAPEHTGK; SEQ ID NO:749) via a lysinamide linkage, as well as FAM-HNP401, which comprises FAM conjugated to the C-terminal cysteine of acetylated HNP401 with GCC linker (Ac-SGQVPWEEPYYVVKKSSGGC-CONH₂: SEQ ID NO:748). See '054 Application; Hingorani et al. 2018, Theranostics 8, 4226-4237; Whitney et al. 2011, Nat. Biotechnol. 29, 352-356.

Further studies showed fluorescent labeling of human (and rodent) resected nerve tissues following topical administration of FAM-NP41, FAM-HNP401, and other conjugates, including N-terminal and C-terminal deletion variants of HNP401, corresponding to SEQ ID NOS: 20, 22-24, and 25-28. Whitney et al. 2011, Nat. Biotechnol. 29, 352-356; Wu et al. 2011, Laryngoscope 121, 805-810; Hussain et al. 2015, PloS One 10, e0119600; Hingorani et al. 2018, Theranostics 8, 4226-4237.

These investigations, however, have not revealed conjugate properties, such as activity, metabolism and clearance-especially in later stages following administration. In a rodent models, for example, the serum half-life of NP41 (as measured by serum fluorescence) is about ten minutes, indicating rapid clearance of the conjugate from the blood. A similar short half-life (15 minutes) in the blood was observed for FAM-HNP401. In contrast, nerve fluorescence of NP41 has a half-life of about 50 minutes and can persist at a low level in tissues for hours. Similarly, fluorescence of FAM-HNP401 was observed in rat sciatic and prostate nerves several hours after intravenous administration. See, e.g., Whitney et al. 2011, Nat. Biotechnol. 29, 352-356; Hingorani et al. 2018, Theranostics 8, 4226-4237.

Details regarding the clearance and subsequent activity of conjugates bound to nerves (or other tissues) are unclear, and it is unknown whether such properties may support other uses for the conjugates in fluorescence guided surgery. The studies described here reveal that nerve-targeting conjugates can confer sensitive and prolonged fluorescent visualization (illumination) of ureters in conjunction with renal excretion. The results support an expanded repertoire for such conjugates as a surgical adjunct. By highlighting nerves, they are useful as guiding agents in surgeries, such as head and neck surgical procedures, where they can help prevent nerve injuries. And as described here, by highlighting ureters, they are useful in other surgeries, such as a many abdominal and pelvic surgical procedures presenting a risk of ureter injuries.

### Results

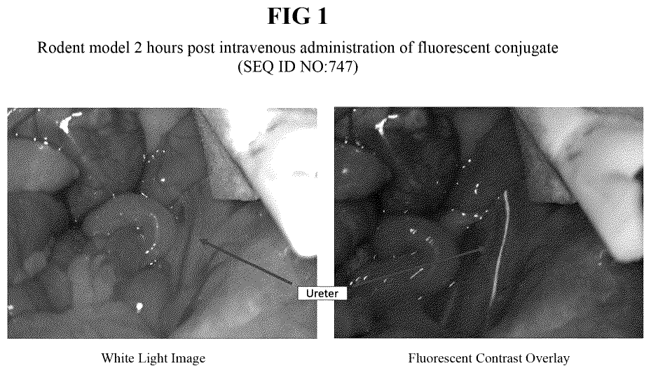

A rodent model was to use to evaluate the dynamics of fluorescent visualization of 5-FAM-QVPWEEPYYVVKKSSGG-NH2 (HNP401-N-2 with GG linker; SEQ ID NO:747) following intravenous administration. The conjugate was administered in a wide range of amounts and fluorescence was detected using a customized microscope. As shown, for example, in FIG. 1, a ureter is readily illuminated 2 hours following intravenous administration of about 2000 nanomoles (˜200 mg/kg) in a mouse model. Additional investigations, exploring a range of amounts from 100 nanomoles to 2000 nanomoles, corresponding to doses from ˜10 mg/kg to ˜200 mg/kg, also revealed ureter illumination following intravenous administration of the fluorescent conjugate, SEQ ID NO:747, in a mouse model. Similar findings were observed in a rat model. Such fluorescent visualization does not, however, reflect specific or direct labeling of the ureter. Rather it reflects fluorescence of urine flowing down the ureter from the kidney to the bladder. Moreover, real time intraoperative imaging reveals ureteral pulsations that concentrate the urine and can enhancing fluorescence visualization of the ureter. Further studies revealed an extended time course of ureter illumination following administration of this conjugate. For example, fluorescent visualization of the ureter was observed in less than 1 hour, as well as up to at least 6 hours, following conjugate administration.

Other investigations in rodent models have demonstrated extended ureter illumination following administration of other conjugates comprising additional peptides and fluorescent moieties. These include molecules consisting of related HNP401 peptides conjugated to FAM, as well as molecules consisting of related HNP401 peptides conjugated to other fluorescent moicties. In addition, the studies presented here allow concurrent visualization of nerves in conjunction with ureters.

### Discussion:

These observations support the general applicability of the nerve targeting fluorescent conjugates described herein in ureter illumination. Accordingly, conjugates disclosed herein can be used to assist surgeons in the visualization of ureters during surgical procedures prior to physically encountering and thus potentially damaging them. Moreover, in the case of inadvertent damage during a surgical procedure, i.e., an iatrogenic injury, such visualization allows for more rapid recognition of the injury and carlier treatment and intervention.

In addition, it has been observed that the administration of the fluorescent conjugates described herein can provide an extended period of ureter visualization following administration. Without being limited by a specific mechanism, this extended period of visualization is believed to reflect the gradual accumulation of fluorescent conjugates in neurons or nerves, such as in the peripheral nervous system, or in other tissues, followed by their gradual release, metabolism and excretion by the urinary system. Thus, in contrast to the systemic administration of a fluorescent dye alone or the direct injection of a fluorescent dye, administering peptide-fluorescent conjugates can advantageously extend the period of ureter illumination, offering both early and longer term illumination, by convenient administration that does not disrupt surgery. In addition, administration of such conjugates can offer concomitant or temporally distinct detection of nerves or ureters.

Such fluorescence-mediated assistance is particularly important during surgical procedures that present a significant risk of ureter injury, particularly iatrogenic ureteral injury (IUI) during surgical procedures. As ureters extend from the abdomen to pelvis, this risk can apply to a broad array of surgical procedures. Applicable procedures that may benefit from ureter illumination include complex abdominal and pelvic surgeries, such as gynecological, urological, and colorectal surgical procedures, as well as cardiovascular surgical procedures. They also include surgical procedures carried out on organs and systems in the vicinity of ureters, such as a kidney, bladder, prostate, uterus, male or female reproductive system, rectum, colon, small intestine, or large intestine. They can also include cancer surgical procedures, such as a prostate cancer surgical procedure, or a colorectal cancer procedure, or procedures in which enhanced visualization of ureters, as well as nerves, is desirable.

### EXAMPLE 2

### Bioanalysis of Fluorescent Conjugates in the Urine of Surgery Patients

Urine samples were collected 30 minutes before intravenous administration of 500 mg of parent test drug (corresponding to the fluorescent-conjugated peptide, SEQ ID NO:747), 30 minutes after infusion prior to head and neck surgery, and 22 hours later. The concentration of intact parent test drug was determined by liquid chromatography with tandem mass spectrometry (LC-MS/MS) using a qualified bioanalytical method. Also evaluated was the presence of metabolites M3-M8 (initially identified from in vitro studies) corresponding to shortened versions of the parent peptide conjugated to the fluorescent moiety. The results are shown in Table 5.

TABLE 5

| | | Test Drug | |
|---|---|---|---|
| Subject | Timepoint | (ng/mL) | Metabolites Present |
| 1 | 30 min | BQL* | |
| 2 | Predose | BQL | |
| | 30 min | 11.5 | M4, M6, M7 |
| | 22 hours | BQL | M4, M6 |
| 3 | Predose | BQL | |
| | 30 min | 19.0 | M4, M6, M7 |
| | 22 hours | BQL | M4, M6 |
| 4 | Predose | BQL | |
| | 30 min | BQL | M4, M6, M7 |
| 5 | Predose | BQL | |
| | 30 min | 10.1 | M4, M6, M7 |

*BQL: Below Quantitation Limit (10 ng/ml)

At the 30-minute time-point, quantifiable levels of test drug (or the fluorescent metabolites M4, M6, and M7) were detected in four of five subjects evaluated. At the 22 hour time point, fluorescent metabolites M4 and M6 were detected in both subjects evaluated. As expected, test drug and fluorescent metabolites were not detected in any predose samples.

These data are consistent with dosing and ureter imaging experiments in the rodent models, indicating that a single administration of test drug can lead to immediate (e.g., 30 minutes) and prolonged accumulation (e.g., 22 hours) of fluorescent molecules in the urine. In turn, this can confer extended ureter visualization at early and later stages in surgical procedures, as well as support concurrent or distinct visualization of nerves and ureters in surgical procedures or other applications.

### EXAMPLE 3

### Ureter and Nerve Illumination During Surgery

The ability of fluorescent conjugates of the present disclosure to image ureters (and nerves) can be evaluated using an appropriately designed clinical study, such as that described below for patients undergoing gastrointestinal/genitourinary (GI/GU) surgery

In this prophetic example, a clinical trial comprises an open-label, multi-center study evaluating administration of a test drug (corresponding to the fluorescent-conjugated peptide, SEQ ID NO: 747) in 80 subjects undergoing GI/GU surgery. Inclusion criteria for enrollment in the study include the requirement that the subject is undergoing a surgical procedure in the GI/GU region where nerves (and ureters) are potentially at risk for injury. For example, such procedures can encompass colorectal or prostate surgery.

Enrolled subjects receive a single dose of test drug (500 mg) by intravenous infusion 2 to 5 hours prior to surgery. Fluorescence of test drug is evaluated by real-time white light reflectance (WLR) and fluorescence (FL) imaging through a laparoscope.

Multiple endpoints are used to assess primary and secondary efficacy objectives.

With respect to nerve detection, the endpoints include a visualization scoring system to measure the efficacy of test drug in improving the contrast enhancement, branching delineation, and length of nerves using WLR alone versus Paired (WLR with FL overlay). They also include computation of signal to background ratio for multiple regions of nerve (and adjacent non-nerve) tissue to measure the efficacy of test drug in improving nerve visualization using WLR versus FL.

With respect to ureter detection, the endpoints include a visualization scoring system to measure the efficacy of test drug in improving the contrast enhancement of ureter using WLR alone versus WLR with FL overlay. They also include computation of signal to background ratio for multiple regions of ureter (and adjacent non-ureter) tissue to measure the efficacy of test drug in improving ureter visualization using WLR versus FL.

A significant improvement in the efficacy of nerve and ureter detection with test drug is shown with multiple endpoints. Improvement is observed at early and later times in the surgical procedures. Accordingly, fluorescent conjugates of the present disclosure can be useful in fluorescent guided GI/GU surgical procedures, including in enhanced visualization of nerves, ureters, or both. Benefits of such enhanced visualization can include significant improvements in surgeon confidence in identifying ureters and nerves in surgical procedures, as well as significant reductions in the duration of such surgical procedures.

The examples set forth above are provided to give those of ordinary skill in the art a complete disclosure and description of how to make and use the embodiments of the compositions and methods of the disclosure, and are not intended to limit the scope of what the inventors regard as their disclosure. Modifications of the above-described modes for carrying out the disclosure that are obvious to persons of skill in the art are intended to be within the scope of the following claims. All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

Furthermore while certain details in the present disclosure are provided to convey a thorough understanding of the invention as defined by the appended claims, it will be apparent to those skilled in the art that certain embodiments may be practiced without these details. Moreover, in certain instances, well-known methods, procedures, or other specific details have not been described to avoid unnecessarily obscuring aspects of the invention defined by the appended claims.

This application claims the benefit of priority to U.S. Provisional Application No. 63/137,621, filed Jan. 14, 2021, which application is hereby incorporated by reference in its entirety.

1. A method of visualizing a ureter in a subject, the method comprising
(a) administering to the subject an effective amount of a fluorescein-conjugated peptide, wherein the fluorescein-conjugated peptide comprises a peptide having the amino acid sequence of SEQ ID NO:21 conjugated to a fluorescein moiety at its N-terminus; and
(b) detecting fluorescence of the fluorescein moiety in a peristaltic urine flow in the ureter (i) after 30 minutes, (ii) within a period of 1 hour or (iii) after 30 minutes and within a period of 1 hour, following administration of the fluorescein-conjugated peptide.

2. The method of claim 1, wherein the fluorescein moiety is carboxyfluorescein.

3. The method of claim 1, wherein the fluorescein moiety is 5-carboxyfluorescein.

4. The method of claim 1, wherein the fluorescein-conjugated peptide is SEQ ID NO:747.

5. The method of claim 1, wherein the subject is a human subject undergoing a surgical procedure.

6. The method of claim 5, wherein the fluorescein-conjugated peptide is first administered prior to the surgical procedure.

7. The method of claim 6, wherein the fluorescein-conjugated peptide is not further administered to the subject following the first administration.

8. The method of claim 5, wherein the surgical procedure comprises a gynecological, urological, colorectal, or cardiovascular surgical procedure.

9. The method of claim 5, wherein the surgical procedure comprises an abdominal or pelvic procedure.

10. The method of claim 5, wherein the surgical procedure is performed on a kidney, bladder, prostate, uterus, male or female reproductive system, rectum, colon, small intestine, or large intestine.

11. The method of claim 5, wherein the surgical procedure is an open surgical procedure, a laparoscopic surgical procedure, a microscopic procedure, or an endoscopic procedure.

12. The method of claim 5, wherein the surgical procedure is a cancer surgical procedure.

13. The method of claim 12, wherein the cancer surgical procedure is a prostate cancer surgical procedure or colorectal cancer surgical procedure.

14. (canceled)

15. The method of claim 1, wherein detecting fluorescence of the ureter occurs for a period of time of more than 1 hour, more than 2 hours, more than 3 hours, more than 4 hours, more than 5 hours, more than 6 hours, more than 7 hours, or more than 8 hours following administration of the fluorescein-conjugated peptide.

16.-18. (canceled)

19. The method of claim 1, wherein the fluorescein-conjugated peptide is administered to the subject intravenously.

20. The method of claim 1, wherein the fluorescein-conjugated peptide is administered to the subject orally.

21. The method of claim 1, wherein the fluorescein-conjugated peptide is in a pharmaceutical composition further comprising a pharmaceutically acceptable excipient.

22. The method of claim 1, further comprising visualizing a nerve in the subject that is labeled by the fluorescein-conjugated peptide.

23. The method of claim 22, wherein the nerve and the ureter are visualized concurrently in the subject during surgery.

24. The method of claim 22, wherein the nerve and the ureter are visualized at different times in the subject during surgery.

25. (canceled)

26. The method of claim 5, wherein detecting fluorescence of the ureter occurs for a period of time of more than 1 hour following administration of the fluorescein-conjugated peptide.

27. The method of claim 5, wherein detecting fluorescence of the ureter occurs after 30 minutes following administration of the fluorescein-conjugated peptide.

28. (canceled)

29. The method of claim 5, wherein the fluorescein-conjugated peptide is administered to the subject intravenously.

30. The method of claim 5, further comprising visualizing a nerve in the subject that is labeled by the fluorescein-conjugated peptide.

31. The method of claim 5, wherein the subject has an iatrogenic ureteral injury (IUI).

32. The method of claim 5, wherein the subject has a ureteral injury arising from ligation, angulation, transection, laceration, crush, ischemia, or resection.

33. A method of reducing injury to a ureter, the method comprising
(a) identifying a subject at a risk of a ureteral injury prior to a surgical procedure
(b) administering to the subject undergoing the surgical procedure an effective amount of a fluorescein-conjugated peptide, wherein the fluorescein-conjugated peptide comprises a peptide having the amino acid sequence of SEQ ID NO:21 conjugated to a fluorescein moiety at its N-terminus; and
(c) visualizing the ureter by detecting fluorescence of the fluorescein moiety in a peristaltic urine flow in the ureter, wherein detecting fluorescence occurs after 30 minutes, within a period of 1 hour, after 30 minutes and within a period of 1 hour, after 1 hour, after 2 hours, after 3 hours, after 4 hours, after 5 hours, after 6 hours, after 7 hours, or after more than 8 hours following administration of the fluorescein-conjugated peptide.

34. The method of claim 33, wherein the ureteral injury is an injury that arises from ligation, angulation, transection, laceration, crush, ischemia, or resection.

35. The method of claim 33, wherein the ureteral injury is an iatrogenic ureteral injury (IUI).
